(19) United States
(12) Patent Application Publication
(10) Pub. No.: US 2005/0266409 A1
(43) Pub. Date: Dec. 1, 2005

(54) Compositions and methods for diagnosing, preventing, and treating cancers

(75) Inventors: Eugene Brown, Newton Highlands, MA (US); Wei Liu, Sudbury, MA (US)

Correspondence Address: NIXON PEABODY, LLP, 401 9TH STREET, NW, SUITE 900, WASHINGTON, DC, 20004-2128, US

(73) Assignee: Wyeth, Madison, NJ (US)

(21) Appl. No.: 10/770,726

(22) Filed: Feb. 4, 2004

Related U.S. Application Data
(60) Provisional application No. 60/444,637, filed on Feb. 4, 2003.

(51) Int. Cl. C12Q 1/68; G06F 19/00; G01N 33/48; G01N 33/50

(52) U.S. Cl. 435/6; 702/20

(57) ABSTRACT

Compositions and methods for diagnosing, preventing, and treating cancers. In one embodiment, genes differentially expressed in colon, lung, breast and prostate cancer tissues relative to corresponding cancer-free tissues are identified. These genes or their products can be used as markers for the detection of respective cancers. Modulators of these genes or their products can be used for the treatment or prevention of respective cancers.

### REFERENCE TO RELATED APPLICATION

This application incorporates by reference all materials recorded in compact discs labeled “Copy 1” and “Copy 2.” Each of the compact discs includes the file entitled “AM101079 Sequence Listing.ST25.txt” (7,373 KB, created on Feb. 2, 2004).

### TECHNICAL FIELD

The present invention relates generally to the diagnosis, prevention, and treatment of cancers, such as colon, lung, breast, or prostate cancers. In one embodiment, this invention employs cancer-related protein kinase genes (CPKGs), or polynucleotides or polypeptides encoded thereby, for the diagnosis, prevention, or treatment of cancers.

### BACKGROUND OF THE INVENTION

Cancer is a significant health problem throughout the world. The most frequently diagnosed cancers include colon cancer, lung cancer, breast cancer, and prostate cancer.

Colon Cancer

Colon cancer is the second most frequently diagnosed malignancy in the United States and is the second most common cause of cancer death. In 2001, there were about 135,400 newly diagnosed cases of colon cancer, with an estimated 56,700 deaths. Although the five-year survival rate for patients with colon cancer detected in an early localized stage is 92%, only 37% of colon cancer is diagnosed at this stage however. The survival rate drops to 64% if the cancer is allowed to spread to adjacent organs or lymph nodes, and to 7% in patients with distant metastases.

The prognosis of colon cancer is directly related to the degree of penetration of the tumor through the bowel wall and the presence or absence of nodal involvement. As a consequence, early detection and treatment are particularly important. Colon cancer typically originates in the colonic epithelium and is not extensively vascularized (non-invasive) during the early stages of development. The transition to a highly-vascularized, invasive and ultimately metastatic cancer commonly takes ten years or longer. With early detection and diagnosis, colon cancer may be effectively treated by, for example, surgical removal of the cancerous or precancerous tissue. However, colon cancer is often detected only upon manifestation of clinical symptoms, such as pain and black tarry stool. Generally, such symptoms are present only when the disease is well established, and only after metastasis has occurred. Therefore, early detection of colon cancer is critically important to significantly reduce its morbidity. Currently, the best means of preventing colon cancer is by early detection of pre-neoplastic lesions in the colon via various invasive and noninvasive screening techniques.

Lung Cancer

Lung cancer is the leading cancer killer in both men and women. In 2000, there were an estimated 164,100 new cases of lung cancer and an estimated 156,900 deaths from lung cancer in the United States. The five-year survival rate among all lung cancer patients, regardless of the stage of disease at diagnosis, is only 13%. This contrasts with a five-year survival rate of 46% among cases detected while the disease is still localized. However, only 16% of lung cancers are discovered before the disease has spread.

Two major types of lung cancer include non-small cell lung cancer and small cell lung cancer. Non-small cell lung cancer is much more common and usually spreads to different parts of the body more slowly than small cell lung cancer. Examples of non-small cell lung cancer include squamous cell carcinoma, adenocarcinoma, and large cell carcinoma. Small cell lung cancer accounts for about 20% of all lung cancers.

Early lung cancer detection is difficult since clinical symptoms are often not seen until the disease has reached an advanced stage. Currently, diagnosis is aided by the use of chest x-rays, sputum analysis of particular cell types, and fiber optic examination of bronchial passages.

Breast Cancer

Breast cancer is a major cause of cancer-related deaths of women in North America. Although advances have been made in its detection and treatment, breast cancer remains to be the second leading cause of cancer-related deaths in women, affecting more than 180,000 women in the United States each year.

Approximately 10% of all breast cancers are currently classified as strongly familial, with many of these appearing to be due to mutations of the hereditary breast cancer genes, BRCA1 or BRCA2. However, at least one-third of breast cancers which seem to run in families are not linked to BRCA1 or BRCA2, suggesting the existence of an additional hereditary breast cancer gene or genes. Recently, structural and functional studies of cancer cell lines and tissues have demonstrated the involvement of many genetic loci and genes in the development of human breast cancer. Cytogenesis and loss of heterozygozity (LOH) studies have led to the discoveries of alterations in human chromosomes including 1p, 1q, 3p, 6q, 7q, 11p, 13q, 16q, 17p, 17q, and 18q, at frequencies as high as 20-60%. Thus, multiple genes are involved in the development of extensively heterogeneous breast cancers.

Diagnosis of the disease currently relies on a combination of routine breast screening procedures and a variety of prognostic parameters, including the analysis of specific tumor markers. However, the use of established markers often leads to a result that is difficult to interpret.

Prostate Cancer

Other than non-melanoma skin cancers, prostate cancer is the most common cancer afflicting American men. In 2000, the American Cancer Society estimated that over 180,000 new cases were diagnosed with prostate cancer in the U.S. alone and that nearly 32,000 people would die from the disease. Prostate cancer is second only to lung cancer as the leading cause of cancer deaths in men, accounting for roughly 11%.

Prostate cancer is a malignant tumor growth within the prostate gland. Its cause is unknown, although high dietary fat intake and increased testosterone levels are believed to be contributory factors. A letter scale (“A” through “D”), which accounts for the location of the cancer, is commonly used to classify the stage of disease. In Stage A, the tumor is not palpable but is detectable in microscopic biopsy. Stage B is characterized by a palpable tumor confined to the prostate. In Stage C, the tumor extends locally beyond the prostate with no distant metastasis. In Stage D, the cancer has spread to the regional lymph nodes or has produced distant metastasis.

Early prostate cancer usually causes no symptoms and can be detected by prostate-specific antigen (PSA) test and/or direct rectal examination (DRE). Advanced prostate cancers often result in hematuria, impotence, and pain in pelvic bone, spine, hips, or ribs. Other diseases can also cause these same symptoms. A core needle biopsy is the main method used to diagnose prostate cancer.

The development of prostate cancer has been linked to several genetic segments. For example, using tumor-derived CREF-Trans 6 cells and differential RNA display, a putative oncogene, prostate tumor inducing gene-1 (PTI-1), was identified (Shen et al., Proc. Natl. Acad Sci USA, 92:6788-6782, 1995). PTI-1 encodes a mutated and truncated human elongation factor-1α (EF-1 α). Normal EF-1α plays a prominent role in protein translation, a process that is critical in controlling gene expression and regulating cell growth. PTI-1 expression is observed in human prostate cancer cell lines (LNCaP, DU-145 and PC-3) and patient-derived prostate carcinoma tissue samples, but not in normal prostate or BPH tissue. This observation suggests that PTI-1 expression may be related specifically to carcinoma development. In addition, the observation that PTI-1 expression also occurs in a high proportion of carcinoma cell lines of the breast, colon and lung indicates that this genetic alteration may be a common event in carcinogenesis.

Protein Kinases

It is estimated that more than 1,000 of the 10,000 proteins active in a typical mammalian cell are phosphorylated. The high energy phosphate, which drives activation, is generally transferred from adenosine triphosphate molecules (ATP) to a particular protein by protein kinases and removed from that protein by protein phosphatases.

The presence or absence of a phosphate moiety modulates protein function in multiple ways. A common mechanism includes changes in the catalytic properties (Vₘₐₓ and Kₘ) of an enzyme, leading to its activation or inactivation.

A second widely recognized mechanism involves promoting protein-protein interactions. An example of such mechanism is the tyrosine autophosphorylation of the ligand-activated EGF receptor tyrosine phosphatase. This event triggers the high-affinity binding to the phosphotyrosine residue on the receptor's C-terminal intracellular domain to the SH2 motif of the adaptor molecule, Grb2. Grb2, in turn, binds through its SH3 motif to a second adaptor molecule, for example, SHC. The formation of this ternary complex activates the signaling events that are responsible for the biological effects of EGF. Serine and threonine phosphorylation events also have been recognized to exert their biological function through protein-protein interaction events that are mediated by the high-affinity binding of phosphoserine and phosphothreonine to WW motifs present in a large variety of proteins.

A third important outcome of protein phosphorylation is changes in the subcellular localization of the substrate. As an example, nuclear import and export events in a large diversity of proteins are regulated by protein phosphorylation.

Protein phosphorylation/dephosphorylation plays a central role in the regulation of a variety of cell functions, such as cell proliferation, differentiation, and cellular signal transduction process. Abnormal phosphorylation processes have been shown many times to be associated with uncontrolled cellular growth and cancer. Current therapies, which are generally based on a combination of chemotherapy or surgery and radiation, continue to prove inadequate in many cancer patients. Accordingly, there is a need in the art for improved methods for screening, diagnosing, and treating cancers.

### SUMMARY OF THE INVENTION

The present invention is directed to cancer genes which are differentially expressed in at least two types of cancer tissues relative to corresponding cancer-free tissues. In many embodiments, the two types of cancer tissues are selected from colon cancer tissue, lung cancer tissue, breast cancer tissue, and prostate cancer tissue. In some embodiments, the cancer genes include cancer-related protein kinase genes (CPKGs), such as those depicted in Table 1. The polynucleotides (e.g., SEQ ID NOS:1-44) and polypeptides (e.g., SEQ ID NOS:45-88) encoded by these genes are designated herein as cancer-related protein kinase polynucleotides (CPKPNs) and cancer-related protein kinases polypeptides (CPKPPs), respectively.

In one aspect, the present invention provides methods useful for diagnosing or monitoring cancers by comparing the expression levels of one or more cancer genes (e.g., CPKGs) in a biological sample of a subject of interest to reference expression levels of the same genes.

In another aspect, the present invention provides pharmaceutical compositions useful for the treatment of cancers. In one embodiment, the pharmaceutical compositions comprise a pharmaceutically acceptable carrier and at least one of the following: (1) an agent that modulates an activity of a CPKPP; (2) an agent that modulates an activity of a CPKPN; and (3) an agent that modulates the expression of a CPKG. In another embodiment, the pharmaceutical compositions include polynucleotides which encode or comprise RNAs capable of inhibiting or reducing the expression of cancer genes (e.g., CPKGs) by RNA interference or antisense mechanisms.

In another aspect, the present invention provides vaccines for prophylactic or therapeutic uses. In one embodiment, the vaccines are generated using at least one of the following (1) a CPKPP or its variant, and (2) a CPKPN or its variant.

The present invention also features methods of the pharmaceutical compositions or vaccines described above for treating or preventing cancers.

In yet another aspect, the present invention provides methods useful for screening anti-tumor agents or chemicals based on the interactions with CPKPPs, or the effects on the expression of CPKGs.

In still another aspect, the present invention provides biochips useful for diagnosing cancer or screening for agents that inhibit cancer. In many cases, the biochips comprise at least one of the following (1) a CPKPP or its variant, (2) a portion of a CPKPP or its variant, (3) a CPKPN or its variant, (4) a portion of a CPKPN or its variant, and (5) an antibody specific for a CPKPP or its variant.

In addition, the present invention provides kits useful for diagnosing cancers. The kits comprise at least one of the following (1) a polynucleotide probe that can hybridize to a CPKPN under reduced stringent, stringent, or highly stringent conditions, and (2) an antibody capable of specifically binding to a CPKPP.

Furthermore, the present invention provides host cells harboring transfected CPKGs. These cells can be used for the treatment of cancers. The present invention also provides knock-out animals in which the genomic seqeunce of at least one CPKG is disrupted.

Other objects, features and advantages of the present invention will become apparent from the following detailed description. The detailed description and specific examples, while indicating preferred embodiments, are given for illustration only since various changes and modifications within the scope of the invention will become apparent to those skilled in the art from this detailed description. Further, the examples demonstrate the principle of the invention and should not be expected to specifically illustrate the application of this invention to all the examples of infections where it obviously will be useful to those skilled in the prior art.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The drawing is provided for illustration, not limitation.

FIG. 1 depicts the transmembrane hidden Markov model (TMHMM) profile of the polypeptide consisting of an amino acid sequence recited in SEQ ID NO:45.

FIG. 2 depicts the TMHMM profile of the polypeptide consisting of an amino acid sequence recited in SEQ ID NO:46.

FIG. 3 depicts the TMHMM profile of the polypeptide consisting of an amino acid sequence recited in SEQ ID NO:47.

FIG. 4 depicts the TMHMM profile of the polypeptide consisting of an amino acid sequence recited in SEQ ID NO:48.

FIG. 5 depicts the TMHMM profile of the polypeptide consisting of an amino acid sequence recited in SEQ ID NO:49.

FIG. 6 depicts the TMHMM profile of the polypeptide consisting of an amino acid sequence recited in SEQ ID NO:50.

FIG. 7 depicts the TMHMM profile of the polypeptide consisting of an amino acid sequence recited in SEQ ID NO:51.

FIG. 8 depicts the TMHMM profile of the polypeptide consisting of an amino acid sequence recited in SEQ ID NO:52.

FIG. 9 depicts the TMHMM profile of the polypeptide consisting of an amino acid sequence recited in SEQ ID NO:53.

FIG. 10 depicts the TMHMM profile of the polypeptide consisting of an amino acid sequence recited in SEQ ID NO:54.

FIG. 11 depicts the TMHMM profile of the polypeptide consisting of an amino acid sequence recited in SEQ ID NO:55.

FIG. 12 depicts the TMHMM profile of the polypeptide consisting of an amino acid sequence recited in SEQ ID NO:56.

FIG. 13 depicts the TMHMM profile of the polypeptide consisting of an amino acid sequence recited in SEQ ID NO:57.

FIG. 14 depicts the TMHMM profile of the polypeptide consisting of an amino acid sequence recited in SEQ ID NO:58.

FIG. 15 depicts the TMHMM profile of the polypeptide consisting of an amino acid sequence recited in SEQ ID NO:59.

FIG. 16 depicts the TMHMM profile of the polypeptide consisting of an amino acid sequence recited in SEQ ID NO:60.

FIG. 17 depicts the TMHMM profile of the polypeptide consisting of an amino acid sequence recited in SEQ ID NO:61.

FIG. 18 depicts the TMHMM profile of the polypeptide consisting of an amino acid sequence recited in SEQ ID NO:62.

FIG. 19 depicts the TMHMM profile of the polypeptide consisting of an amino acid sequence recited in SEQ ID NO:63.

FIG. 20 depicts the TMHMM profile of the polypeptide consisting of an amino acid sequence recited in SEQ ID NO:64.

FIG. 21 depicts the TMHMM profile of the polypeptide consisting of an amino acid sequence recited in SEQ ID NO:65.

FIG. 22 depicts the TMHMM profile of the polypeptide consisting of an amino acid sequence recited in SEQ ID NO:66.

FIG. 23 depicts the TMHMM profile of the polypeptide consisting of an amino acid sequence recited in SEQ ID NO:67.

FIG. 24 depicts the TMHMM profile of the polypeptide consisting of an amino acid sequence recited in SEQ ID NO:68.

FIG. 25 depicts the TMHMM profile of the polypeptide consisting of an amino acid sequence recited in SEQ ID NO:69.

FIG. 26 depicts the TMHMM profile of the polypeptide consisting of an amino acid sequence recited in SEQ ID NO:70.

FIG. 27 depicts the TMHMM profile of the polypeptide consisting of an amino acid sequence recited in SEQ ID NO:71.

FIG. 28 depicts the TMHMM profile of the polypeptide consisting of an amino acid sequence recited in SEQ ID NO:72.

FIG. 29 depicts the TMHMM profile of the polypeptide consisting of an amino acid sequence recited in SEQ ID NO:73.

FIG. 30 depicts the TMHMM profile of the polypeptide consisting of an amino acid sequence recited in SEQ ID NO:74.

FIG. 31 depicts the TMHMM profile of the polypeptide consisting of an amino acid sequence recited in SEQ ID NO:75.

FIG. 32 depicts the TMHMM profile of the polypeptide consisting of an amino acid sequence recited in SEQ ID NO:76.

FIG. 33 depicts the TMHMM profile of the polypeptide consisting of an amino acid sequence recited in SEQ ID NO:77.

FIG. 34 depicts the TMHMM profile of the polypeptide consisting of an amino acid sequence recited in SEQ ID NO:78.

FIG. 35 depicts the TMHMM profile of the polypeptide consisting of an amino acid sequence recited in SEQ ID NO:79.

FIG. 36 depicts the TMHMM profile of the polypeptide consisting of an amino acid sequence recited in SEQ ID NO:80.

FIG. 37 depicts the TMHMM profile of the polypeptide consisting of an amino acid sequence recited in SEQ ID NO:81.

FIG. 38 depicts the TMHMM profile of the polypeptide consisting of an amino acid sequence recited in SEQ ID NO:82.

FIG. 39 depicts the TMHMM profile of the polypeptide consisting of an amino acid sequence recited in SEQ ID NO:83.

FIG. 40 depicts the TMHMM profile of the polypeptide consisting of an amino acid sequence recited in SEQ ID NO:84.

FIG. 41 depicts the TMHMM profile of the polypeptide consisting of an amino acid sequence recited in SEQ ID NO:85.

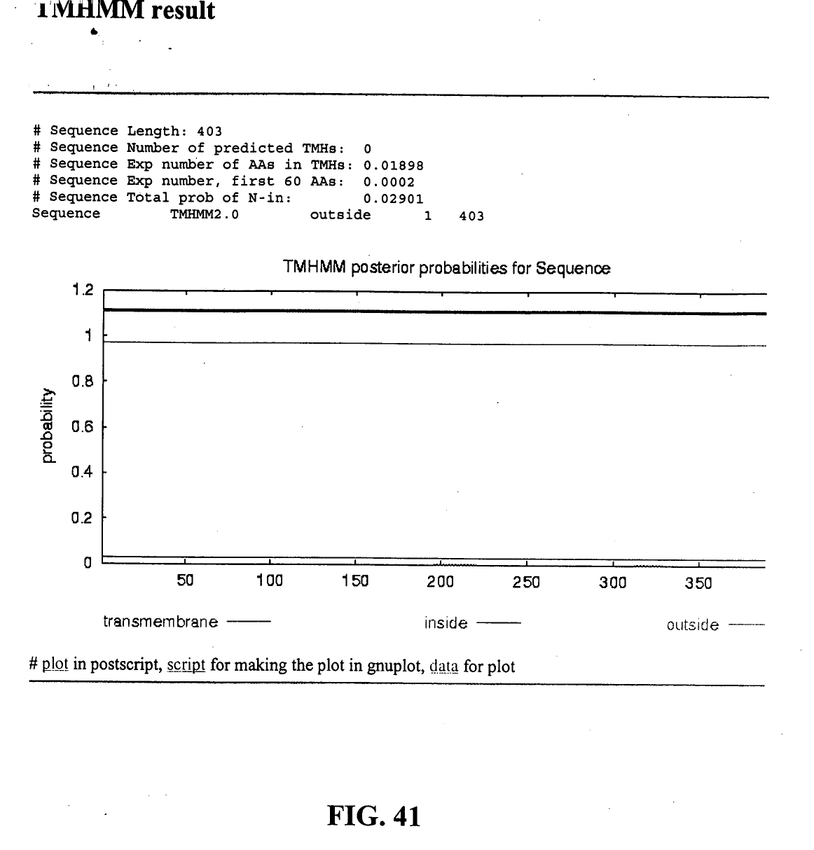

FIG. 42 depicts the TMHMM profile of the polypeptide consisting of an amino acid sequence recited in SEQ ID NO:86

FIG. 43 depicts the TMHMM profile of the polypeptide consisting of an amino acid sequence recited in SEQ ID NO:87.

FIG. 44 depicts the TMHMM profile of the polypeptide consisting of an amino acid sequence recited in SEQ ID NO:88.

### DETAILED DESCRIPTION OF THE INVENTION

The present invention provides compositions and methods for the diagnosis, prevention, or treatment of numerous cancers. The present invention also provides methods for the identification of novel therapeutic agents for treating cancers. In addition, the present invention provides animal models useful for studying the pathogenesis of cancers. The present invention is based on the discovery of cancer genes that are overexpressed in at least two types of cancer tissues as compared to corresponding cancer-free tissues. In many embodiments, the cancer genes are overexpressed in colon cancer, lung cancer, breast cancer, or prostate cancer tissues, as compared to the corresponding cancer-free tissues. In one example, the average expression level of each cancer gene in a cancer tissue is at least 1.5, 2, 3, 4, 5, or more times higher than that in the corresponding cancer-free tissue. In another example, the p-value of Student's t-test for the over-expression of each cancer gene in cancer versus cancer-free tissues is no greater than 0.01, 0.005, 0.001, or less.

In many other embodiments, the cancer genes of the present invention include “cancer-related protein kinase gene (CPKG).” CPKGs are protein kinase genes that are identified by the two-tier statistical analysis of the Gene Logic BioExpress database as showing higher levels of mRNA expression in tumor specimens when compared with the corresponding cancer-free (normal) tissue samples. Examples of the CPKGs of the present invention are shown in Table 1. The mRNA molecules of these CPKGs were found to be up-regulated in tissues from at least two of the four major types of cancers, i.e., colon adenocarcinoma, lung adenocarcinoma, breast infiltrating ductal carcinoma, and prostate adenocarcinoma. The kinases encoded by the CPKGs are referred to as cancer-related protein kinases (CPKs).

TABLE 1
Cancer-Related Protein Kinases

| Gene Symbol | Locuslink | cDNA Sequence | Amino Acid Sequence |
|---|---|---|---|
| ATR | 545 | SEQ ID NO: 1 | SEQ ID NO: 45 |
| BCR | 613 | SEQ ID NO: 2 | SEQ ID NO: 46 |
| BUB1B | 701 | SEQ ID NO: 3 | SEQ ID NO: 47 |
| CDC2 | 983 | SEQ ID NO: 4 | SEQ ID NO: 48 |
| CDC2L1 | 984 | SEQ ID NO: 5 | SEQ ID NO: 49 |
| CDC7L1 | 8317 | SEQ ID NO: 6 | SEQ ID NO: 50 |
| CDK2 | 1017 | SEQ ID NO: 7 | SEQ ID NO: 51 |
| CDK4 | 1019 | SEQ ID NO: 8 | SEQ ID NO: 52 |
| CDK5 | 1020 | SEQ ID NO: 9 | SEQ ID NO: 53 |
| CDK5R1 | 8851 | SEQ ID NO: 10 | SEQ ID NO: 54 |
| CDK7 | 1022 | SEQ ID NO: 11 | SEQ ID NO: 55 |
| CHEK1 | 1111 | SEQ ID NO: 12 | SEQ ID NO: 56 |
| CIT | 11113 | SEQ ID NO: 13 | SEQ ID NO: 57 |
| CKS2 | 1164 | SEQ ID NO: 14 | SEQ ID NO: 58 |
| CSNX2A1 | 1457 | SEQ ID NO: 15 | SEQ ID NO: 59 |
| C20orf97 | 57761 | SEQ ID NO: 16 | SEQ ID NO: 60 |
| EPHB2 | 2048 | SEQ ID NO: 17 | SEQ ID NO: 61 |
| ERBB2 | 2064 | SEQ ID NO: 18 | SEQ ID NO: 62 |
| ERBB3 | 2065 | SEQ ID NO: 19 | SEQ ID NO: 63 |
| GSG2 | 83903 | SEQ ID NO: 20 | SEQ ID NO: 64 |
| GSK3B | 2932 | SEQ ID NO: 21 | SEQ ID NO: 65 |
| IRAK1 | 3654 | SEQ ID NO: 22 | SEQ ID NO: 66 |
| KIAA0175 | 9833 | SEQ ID NO: 23 | SEQ ID NO: 67 |
| MAPKAPK5 | 8550 | SEQ ID NO: 24 | SEQ ID NO: 68 |
| MAPK13 | 5603 | SEQ ID NO: 25 | SEQ ID NO: 69 |
| NEK2 | 4751 | SEQ ID NO: 26 | SEQ ID NO: 70 |
| PAK4 | 10298 | SEQ ID NO: 27 | SEQ ID NO: 71 |
| PCTK1 | 5127 | SEQ ID NO: 28 | SEQ ID NO: 72 |
| PDK3 | 5165 | SEQ ID NO: 29 | SEQ ID NO: 73 |
| PKMYT1 | 9088 | SEQ ID NO: 30 | SEQ ID NO: 74 |
| PLK | 5347 | SEQ ID NO: 31 | SEQ ID NO: 75 |
| PRKCL1 | 5585 | SEQ ID NO: 32 | SEQ ID NO: 76 |
| PRKDC | 5591 | SEQ ID NO: 33 | SEQ ID NO: 77 |
| PTK2 | 5747 | SEQ ID NO: 34 | SEQ ID NO: 78 |
| PTK6 | 5753 | SEQ ID NO: 35 | SEQ ID NO: 79 |
| RIPK2 | 8767 | SEQ ID NO: 36 | SEQ ID NO: 80 |
| RPS6KB2 | 6199 | SEQ ID NO: 37 | SEQ ID NO: 81 |
| SRPK1 | 6732 | SEQ ID NO: 38 | SEQ ID NO: 82 |
| STK6 | 6790 | SEQ ID NO: 39 | SEQ ID NO: 83 |
| STK12 | 9212 | SEQ ID NO: 40 | SEQ ID NO: 84 |
| STK15 | 8465 | SEQ ID NO: 41 | SEQ ID NO: 85 |
| STK18 | 10733 | SEQ ID NO: 42 | SEQ ID NO: 86 |
| STK39 | 27347 | SEQ ID NO: 43 | SEQ ID NO: 87 |
| TTK | 7272 | SEQ ID NO: 44 | SEQ ID NO: 88 |

The following is a brief annotation for each CRKG in Table 1:

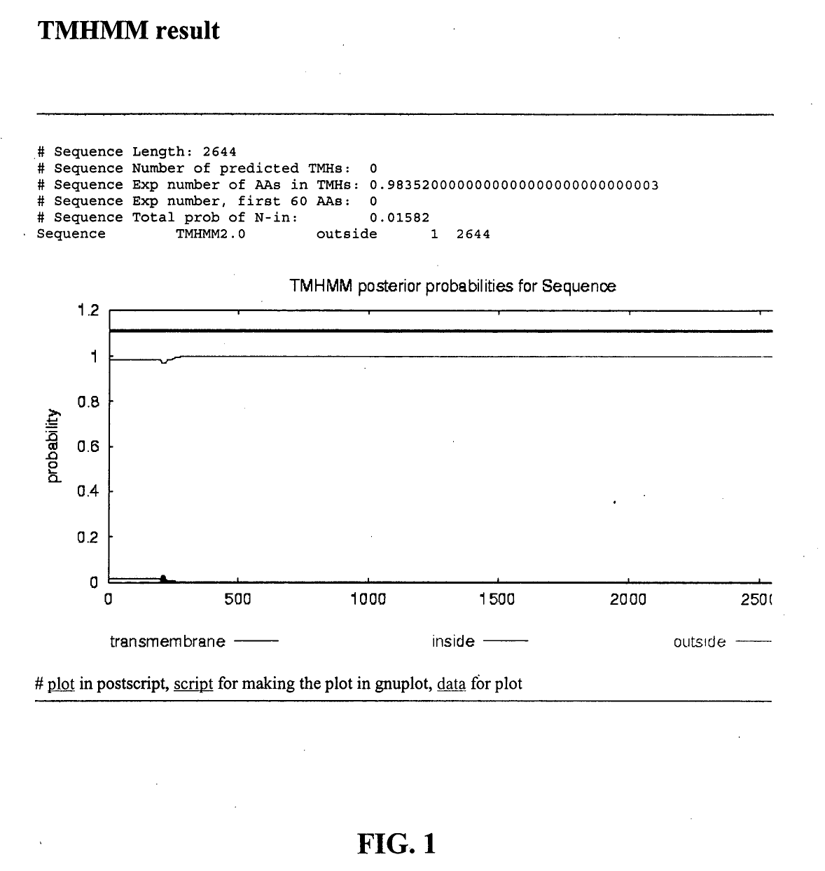

ATR (ataxia telangiectasia and Rad3 related): ATR belongs to the P13/PI4-kinase family, and is most closely related to ATM, a protein kinase encoded by the gene mutated in ataxia telangiectasia. ATR and ATM share similarity with Schizosaccharomyces pombe rad3, a cell cycle checkpoint gene required for cell cycle arrest and DNA damage repair in response to DNA damage. ATR has been shown to phosphorylate checkpoint kinase CHKI, checkpoint proteins RAD17 and RAD9, as well as tumor suppressor protein BRCAL An alternatively spliced transcript variant of ATR gene has been reported, however, its full length nature is not known. Transcript variants utilizing alternative polyA sites exist. ATR is highly expressed in tumor endothelial cells but not in normal endothelial cells. ATR kinase plays an important role during tumor development in responding to hypoxia-induced replication arrest. ATR-deficient mice have been generated. The ATR +/− mice survived nearly as long as ATR +/+ mice but had an increase in tumor incidence. In contrast, and unlike ATM −/− or p53 −/− mice, ATR −/− embryos survived to the blastocyst stage at day 3.5 postcoitum but not to day 7.5. In culture, wild type and heterozygous blastocysts were initially indistinguishable from the ATR −/− cells, but the ATR −/− cells would die due to suffering from chromosomal fragmentation. TUNEL analysis revealed widespread apoptosis after 3 days of culture, and the apoptosis could be blocked by inhibition of Casp3. It was thus speculated that ATR may be particularly essential in the early embryo to sense incomplete DNA replication and prevent mitotic catastrophe. The transmembrane hidden Markov model (TMHMM) profile of ATR is shown in FIG. 1.

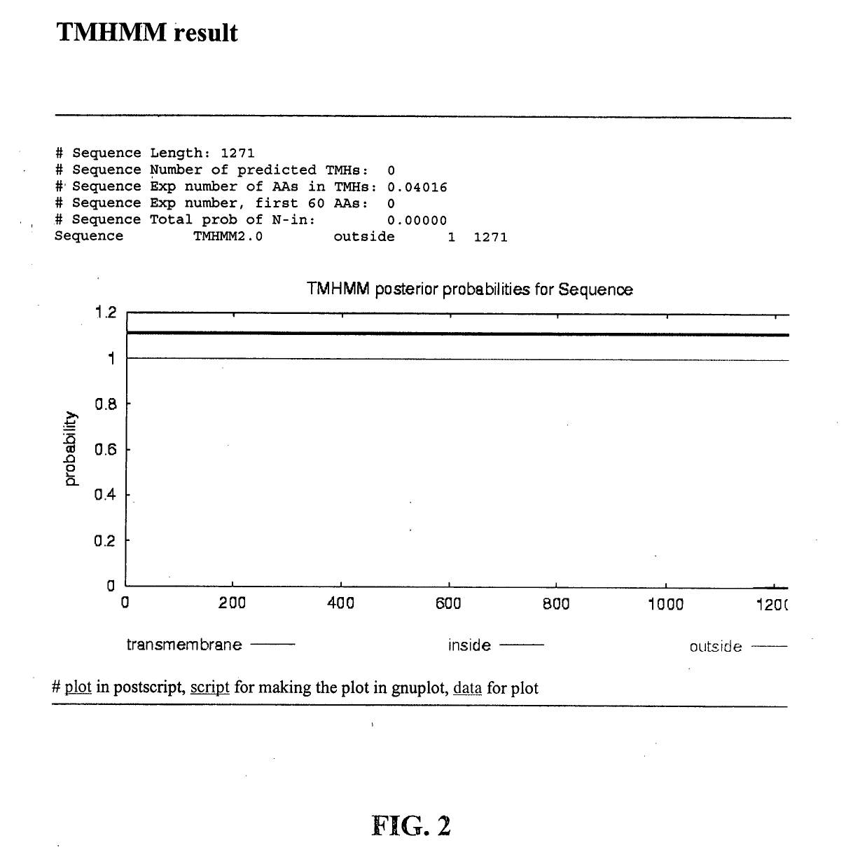

BCR (breakpoint cluster region): A reciprocal translocation between chromosomes 22 and 9 produces the Philadelphia chromosome, which is often found in patients with chronic myelogenous leukemia. The chromosome 22 breakpoint for this translocation is located within the BCR gene. The translocation produces a fusion protein which is encoded by sequence from both BCR and ABL, the gene at the chromosome 9 breakpoint. Although the BCR-ABL fusion protein has been extensively studied, the function of the normal BCR gene product is not clear. The protein has ser/thr kinase activity and is a GTPase-activating protein for p21 rac. The TMHMM profile of BCR is shown in FIG. 2.

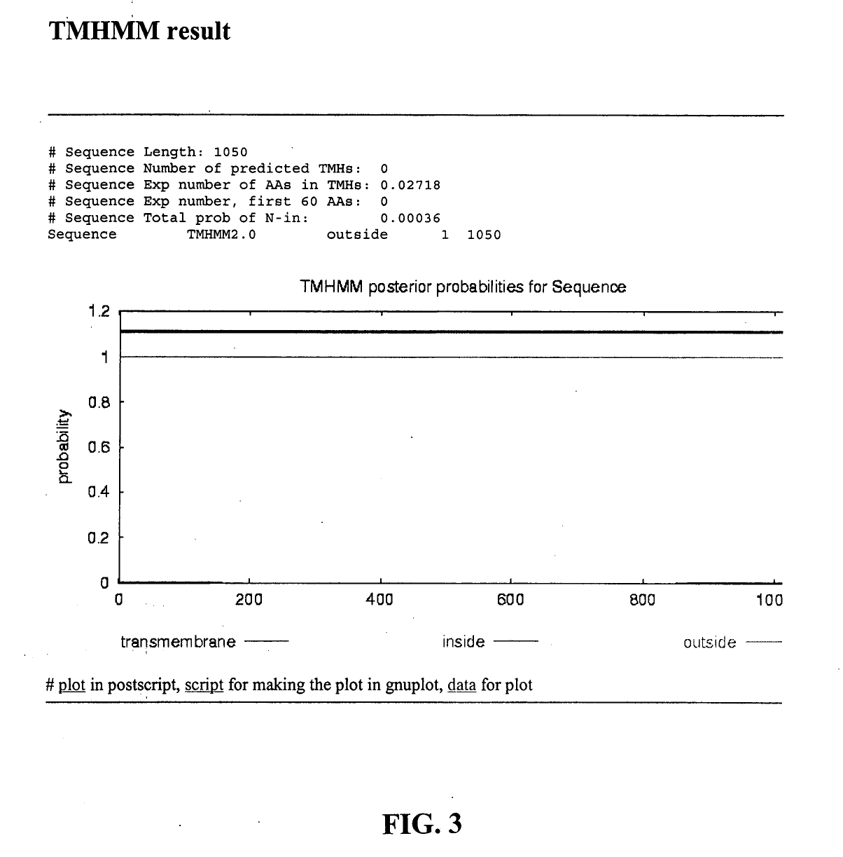

BUBIB (budding uninhibited by benzimidazoles 1 (yeast homolog), beta): BUBI is a protein kinase that makes up the mitotic spindle checkpoint and is required for normal mitotic progression. The predicted BUBIB protein contains the conserved CD1 and CD2 domains that are found in yeast, human, and mouse BUB1; the human and mouse BUBIB proteins are 29% identical in these regions. CD1 directs kinetochore localization and binding to BUB3, and CD2 contains the kinase domain. BUB1 gene expression was detected in 19 colorectal cancer cell lines showing a chromosome instability (CIN) phenotype. A mutation in the BUB1 gene was detected in two of these cell lines. The TMHMM profile of BUBIB is shown in FIG. 3.

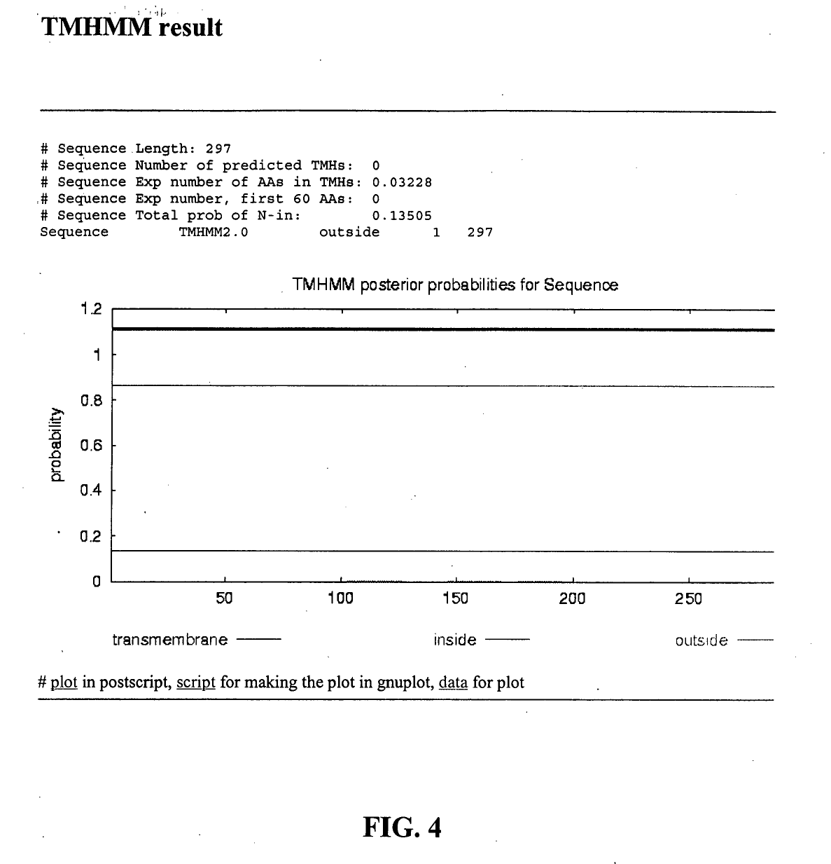

CDC2: This is a ser/thr protein kinase that regulates entry into mitosis. It is the catalytic subunit of the highly conserved protein kinase complex known as M phase promoting factor, which is essential for G1/S and G2/M phase transitions. Unlike yeast CDC2, the mammalian counterpart appears to be transcriptionally regulated. Its phosphorylation provides a second level of regulation. The TMHMM profile of CDC2 is shown in FIG. 4.

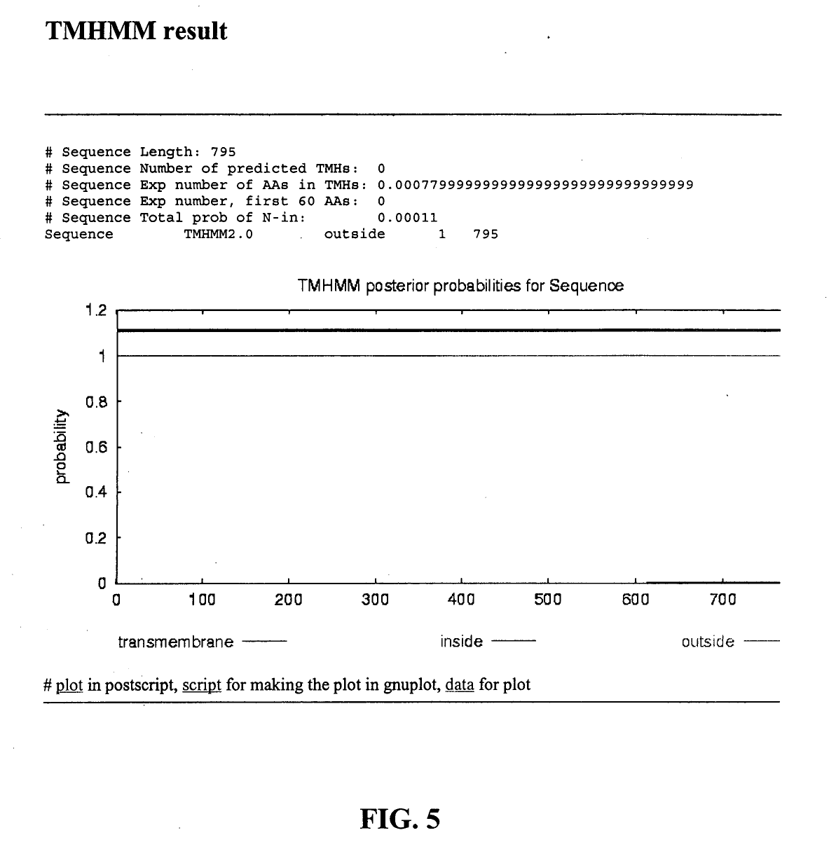

CDC21J1 (cell division cycle 2-like 1 (PITSLRE proteins)): CDC21J1 is a member of the p34Cdc2 protein kinase family. P34Cdc2 kinase family members are known to be essential for eukaryotic cell cycle control. The CDC21J1 gene is in close proximity to the CDC2L2 gene, a nearly identical gene in the same chromosomal region. The gene loci including this gene, CDC2L2, as well as metalloprotease MMP21/22, consist of two identical, tandemly linked genomic regions which are thought to be a part of the larger region that has been duplicated. The CDC21J1 and CDC2L2 genes were shown to be deleted or altered frequently in neuroblastoma with amplified MYCN genes. CDC21J1 could be cleaved by caspases and was demonstrated to play roles in cell apoptosis. A large number of the alternatively spliced variants of the CDC21J1 gene has been reported. The TMHMM profile of CDC21J1 is shown in FIG. 5.

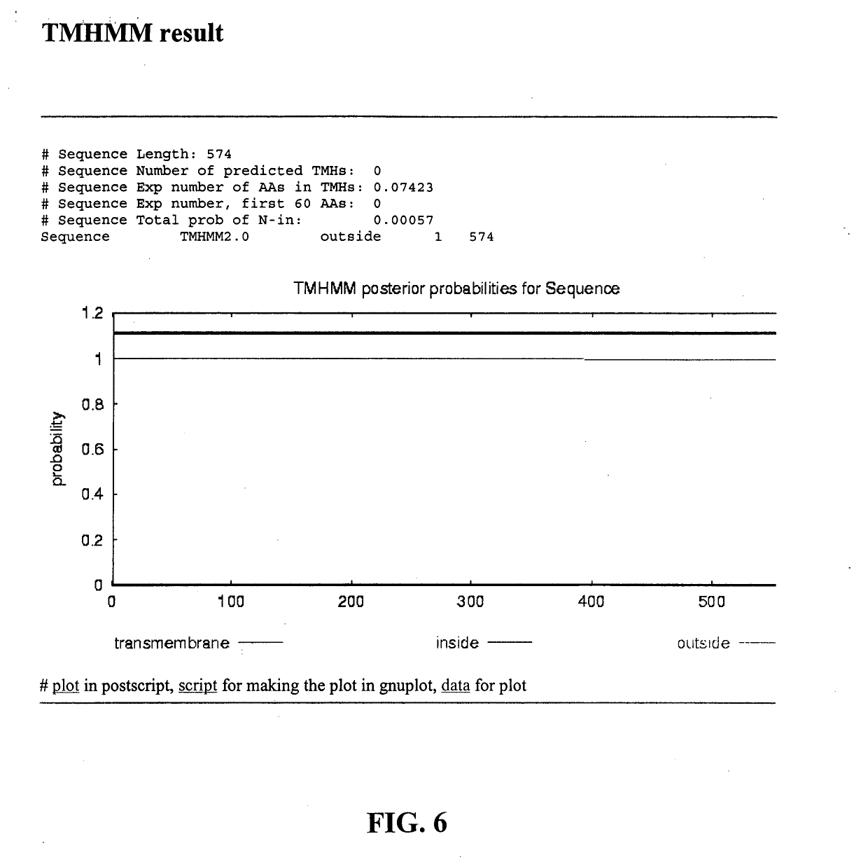

CDC7L1 (CDC7 cell division cycle 7-like 1, S. cerevisiae): CDC7L1 is predominantly localized in the nucleus and is a cell division cycle protein with kinase activity. Although expression levels of the protein appear to be constant throughout the cell cycle, the protein kinase activity appears to increase during S phase. It has been suggested that the protein is essential for initiation of DNA replication and that it plays a role in regulating cell cycle progression. Overexpression of this gene product may be associated with neoplastic transformation for some tumors. Additional transcript sizes have been detected, suggesting the presence of alternative splicing. CDC21J1 is a ser/thr kinase and phosphorylates histone H1 and Mcm proteins in vitro. It is similar to S. cerevisiae CDC7p. The TMHMM profile of CDC7L1 is shown in FIG. 6.

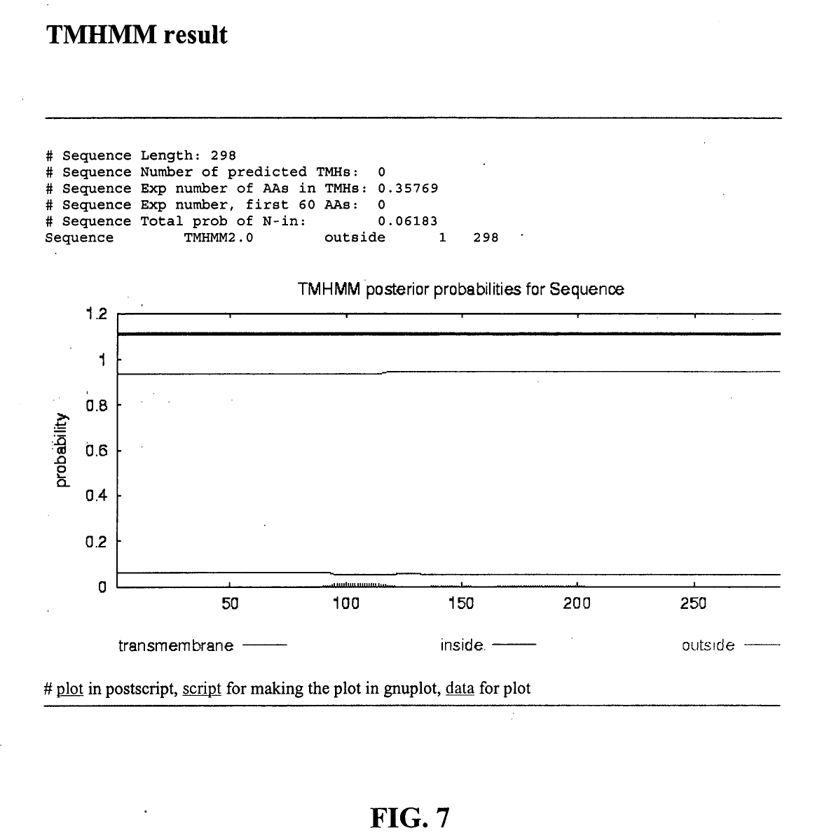

CDK2 (cyclin-dependent kinase 2): CDK2 is a member of the ser/thr protein kinase family. This protein kinase is highly similar to the gene products of S. cerevisiae CDC28, and S. pombe CDC2. It is a catalytic subunit of the cyclin-dependent protein kinase complex, whose activity is restricted to the Gl-S phase and is essential for cell cycle GUS phase transition. This protein associates with and regulated by the regulatory subunits of the complex including cyclin A or E, CDK inhibitor p21Cip1 (CDKNIA) and p27Kip1 (CDKNIB). Its activity is also regulated by protein phosphorylation. Phosphorylation at thr14 or tyr15 inactivates the enzyme, while phosphorylation at thr160 activates it. Two alternatively spliced variants and multiple transcription initiation sites of the CDK2 gene have been reported. CDK2 is associated with cyclin A and cyclin E, and is involved in promoting DNA synthesis and cell cycle progression. The N-terminal domain of CDK2 interacts with cyclins, while the C-terminal domain interacts with CSK1. It is probably involved in the control of the cell cycle. The TMHMM profile of CDK2 is shown in FIG. 7.

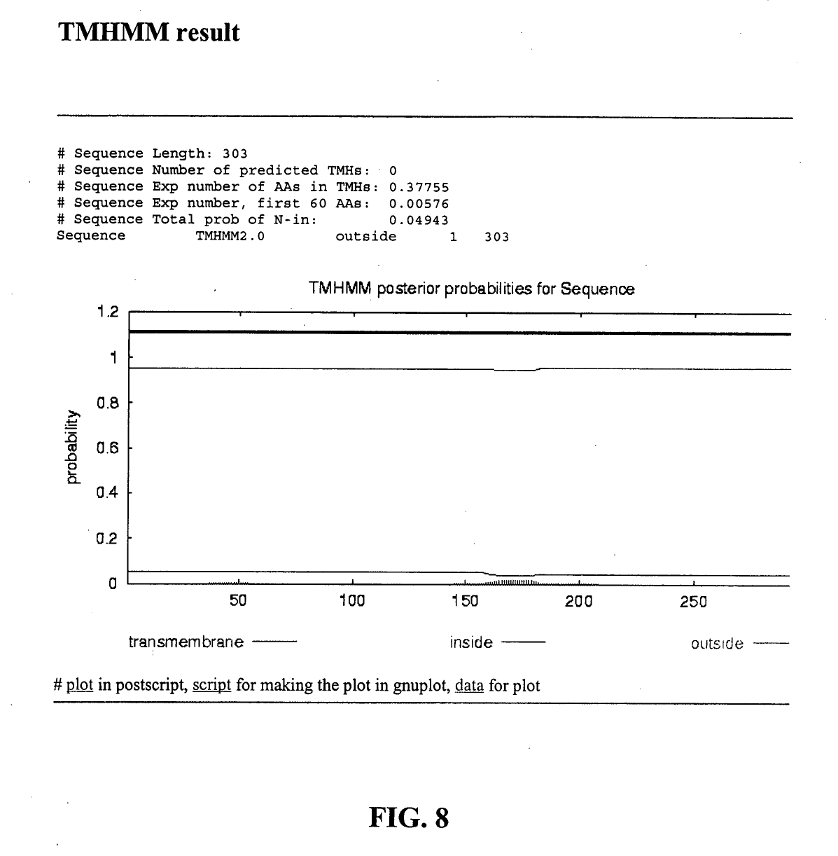

CDK4 (cyclin-dependent kinase 4): The protein encoded by this gene is a member of the ser/thr protein kinase family. This protein is highly homologous to the gene products of S. cerevisiae CDC28, and S. pombe CDC2. It is a catalytic subunit of the protein kinase complex that is important for cell cycle G1 phase progression. The activity of this kinase is restricted to the G_{1/}S phase, which is controlled by the regulatory subunits D-type cyclins and CDK inhibitor p16 (INK4a). This kinase was shown to be responsible for the phosphorylation of retinoblastoma gene product (Rb). The mutations in this gene, as well as its related proteins including D-type cyclins, pl6(INK4a) and Rb, were all found to be associated with tumorigenesis of a variety of cancers. Two alternatively spliced variants and multiple polyadenylation sites of this gene have been reported. The TMHMM profile of CDK4 is shown in FIG. 8.

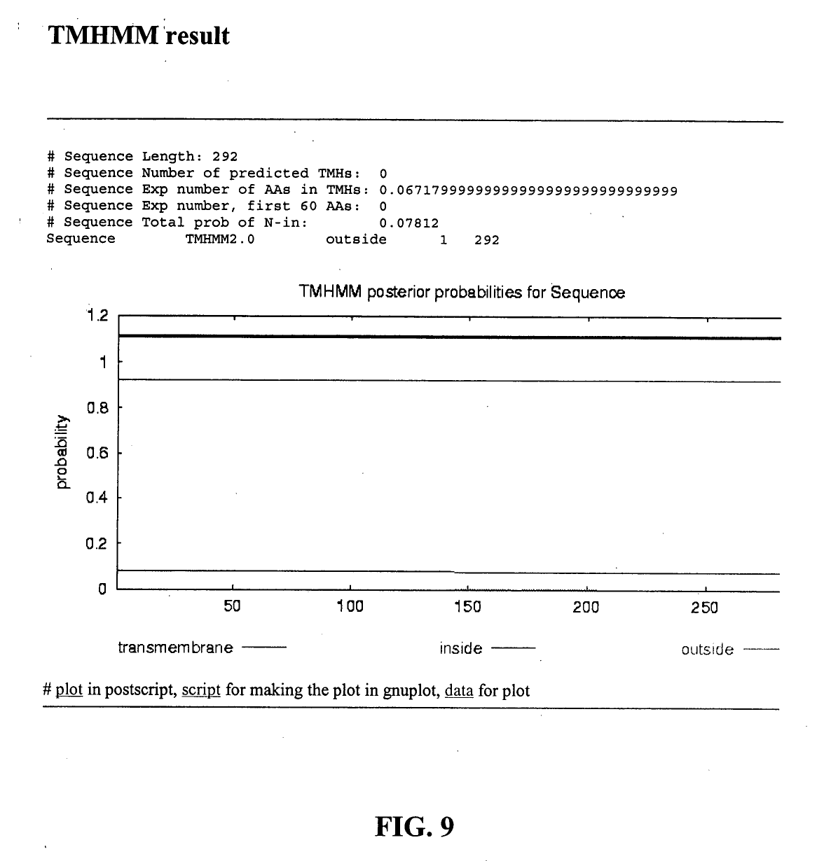

CDK5 (cyclin-dependent kinase 5): CDK5 interacts with a non-cyclin regulatory subunit CDK5R1 and is strongly similar to murine CDK5. The p34Cdc2 protein kinase regulates important transitions in the eukaryotic cell cycle. cDNAs encoding 7 novel human protein kinases were identified using RT-PCR of HeLa cell mRNA with degenerate primers corresponding to conserved regions of CDC2. One of these proteins is CDK5 (also designated PSSALRE, following the accepted practice of naming CDC2-related kinases based on the amino acid sequence of the region corresponding to the conserved PSTAIRE motif of CDC2). The predicted 291-amino acid CDK5 protein shares 57% identity with CDC2. The in vitro transcription/translation product of the CDK5 has an apparent molecular weight of 31 kd by SDS-PAGE. Northern blot analysis detected CDK5 expression in all human tissues and cell lines tested. CDK5-null mice were also generated and were found to exhibit unique lesions in the central nervous system, which is associated with perinatal mortality. The brains of CDK5-null mice lacked cortical laminar structure and cerebellar foliation. In addition, the large neurons in the brain stem and in the spinal cord showed chromatolytic changes with accumulation of neurofilament immunoreactivity. It thus appears that CDK5 is an important molecule for brain development and neuronal differentiation and that CDK5 may play critical roles in neuronal cytoskeleton structure and organization. The TMHMM profile of CDK5 is shown in FIG. 9.

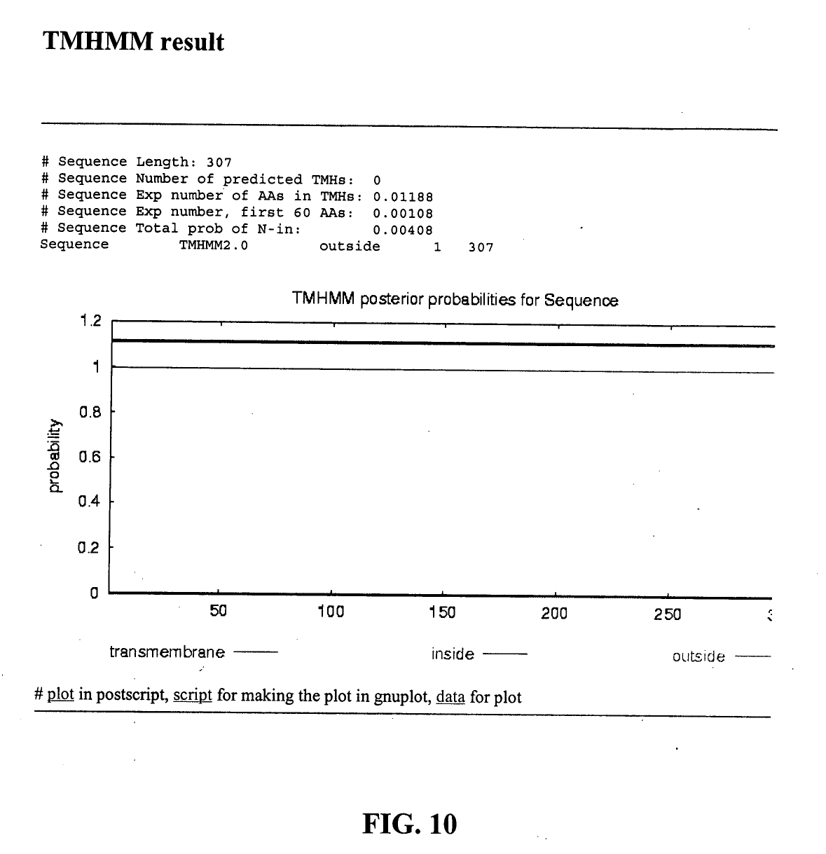

CDK5R1 (cyclin-dependent kinase 5, regulatory subunit 1 (p35)): CDK5R1 is a neuron-specific activator of CDK5, whose activation is required for proper development of the central nervous system. A truncated form of CDK5R1 is found to be accumulated in the brain neurons of patients with Alzheimer's disease. Accumulation of the truncated protein could lead to the deregulation of CDK5, and consequently create aberrantly phosphorylated forms of the microtubuleassociated protein tau, which contributes to patients with Alzheimer's disease. CDK5R1 is not a cyclin family member and is strongly similar to rat Rn11213. The TMHMM profile of CDK5R1 is shown in FIG. 10.

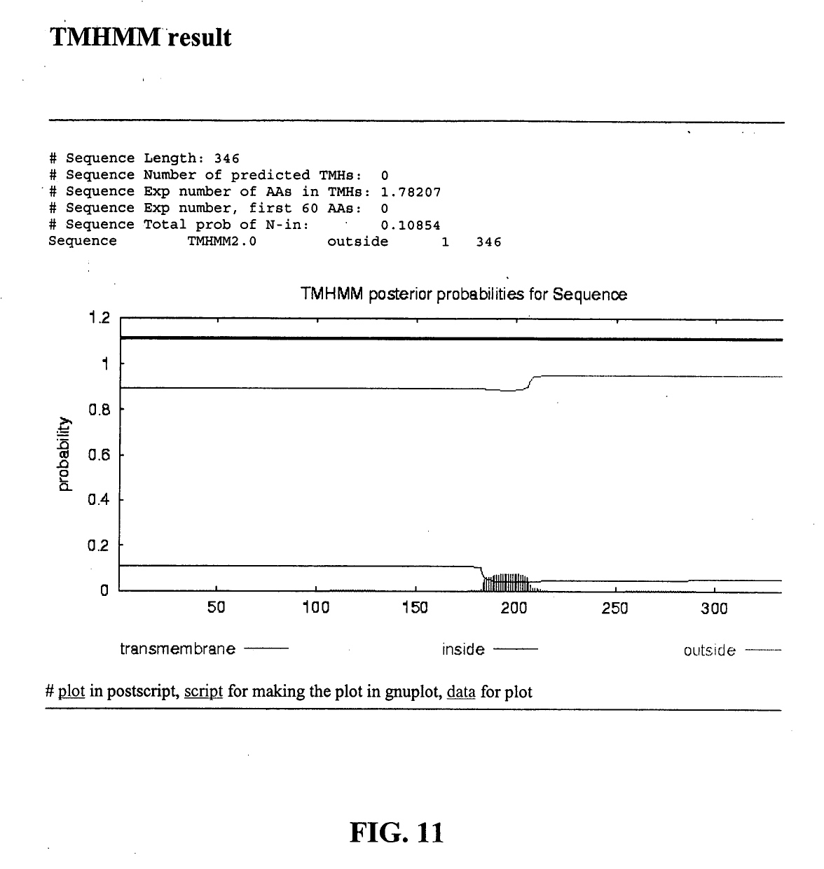

CDK7 (cyclin-dependent kinase 7): CDK7 is a member of the cyclin-dependent protein kinase (CDK) family. CDK family members are highly similar to the gene products of S cerevisiae CDC28, and Spombe CDC2, and are known to be important regulators of cell cycle progression. CDK7 forms a trimeric complex with cyclin H and MAT1, which functions as a CDK-activating kinase (CAK). It is an essential component of the transcription factor TFIIH, which is involved in transcription initiation and DNA repair. This protein is thought to serve as a direct link between the regulation of transcription and the cell cycle. The TMHMM profile of CDK7 is shown in FIG. 11.

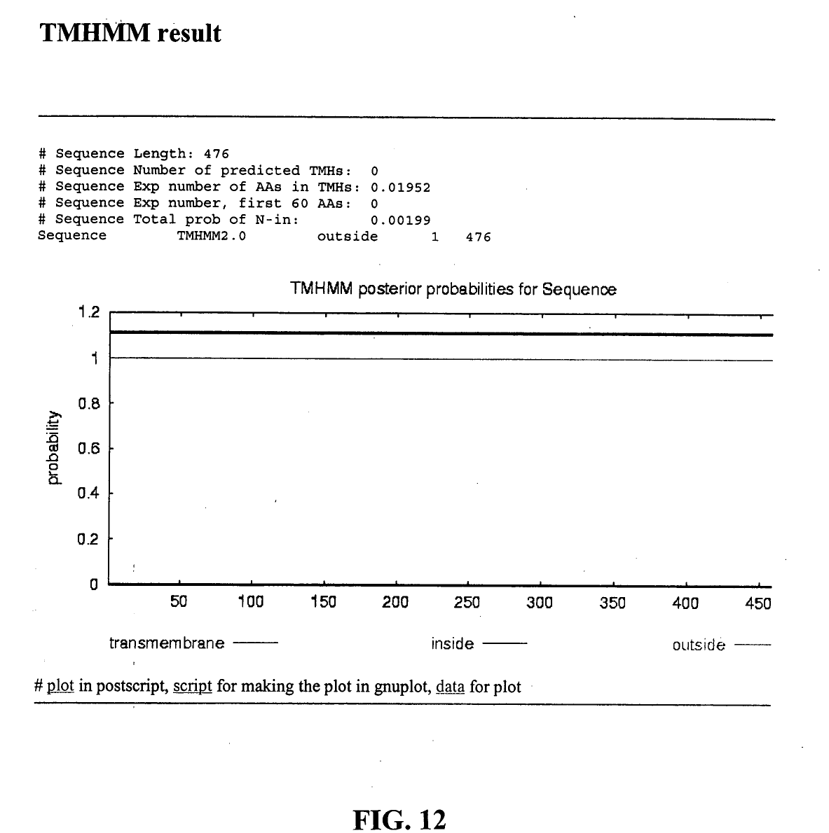

CHEK1 (CHK1 checkpoint homolog, S. pombe): This protein kinase inhibits mitotic entry after DNA damage. It is required for the DNA damage checkpoint. In vitro, CHK1 directly phosphorylates a regulator of CDC2 tyrosine phosphorylation, CDC25C. It has been proposed that, in response to DNA damage, CHK1 phosphorylates and inhibits CDC25C, thus preventing activation of the CDC2-cyclin B complex and mitotic entry. Targeted disruption of CHEK1 in mice showed that CHEK1 deficiency is lethal in early embryogenesis. In culture, CHEK1 −/−, but not CHEK1 +/, blastocysts demonstrated a severe defect in outgrowth of the inner cell mass and died of apoptosis, as determined by TUNEL analysis. CHEK1 is also indispensable for cell cycle arrest before mitosis. The TMHMM profile of CHEK1 is shown in FIG. 12.

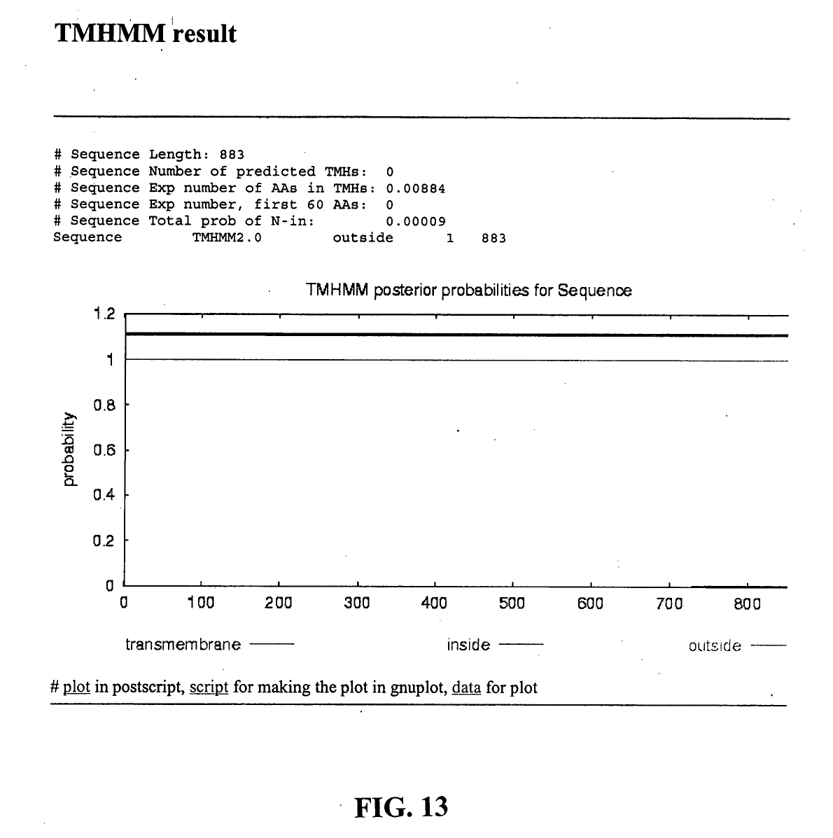

CIT (citron, rho-interacting, ser/thr kinase 21): CIT is a ser/thr kinase in the myotonic dystrophy kinase family. It may or may not contain the sequence of Citron. CIT is a putative rho/rac effector that binds to the GTP-bound forms of rho and rac1. It probably binds p21 with a tighter specificity in vivo. Mice deficient in citron kinase (Citron-K −/− mice), growing at slower rates, are severely ataxic and die before adulthood due to fatal seizures. The brains of the Citron-K −/− mice display defective neurogenesis, with dramatic depletion of microneurons in the olfactory bulb, hippocampus, and cerebellum. These abnormalities arise during development of the central nervous system due to altered cytokinesis and massive apoptosis. It was suggested that citron kinase is essential for cytokinesis in vivo, and in specific neuronal precursors only. Moreover, CIP may be involved in a novel molecular mechanism for a subset of human malformation syndromes of the central nervous system. The TMHMM profile of CIT is shown in FIG. 13.

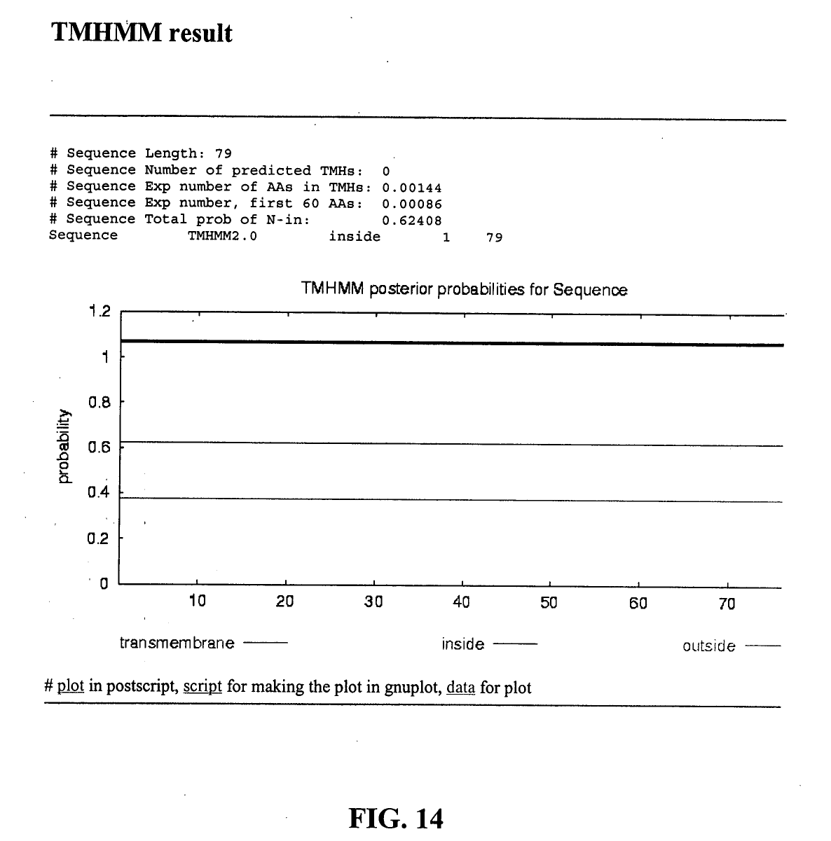

CKS2 (CDC28 protein kinase 2): CKS2 protein binds to the catalytic subunit of the cyclin dependent kinases and is essential for their biological function. The CKS2 mRNA is found to be expressed in different patterns through the cell cycle in HeLa cells, which reflects specialized role for the encoded protein. The TMHMM profile of CKS2 is shown in FIG. 14.

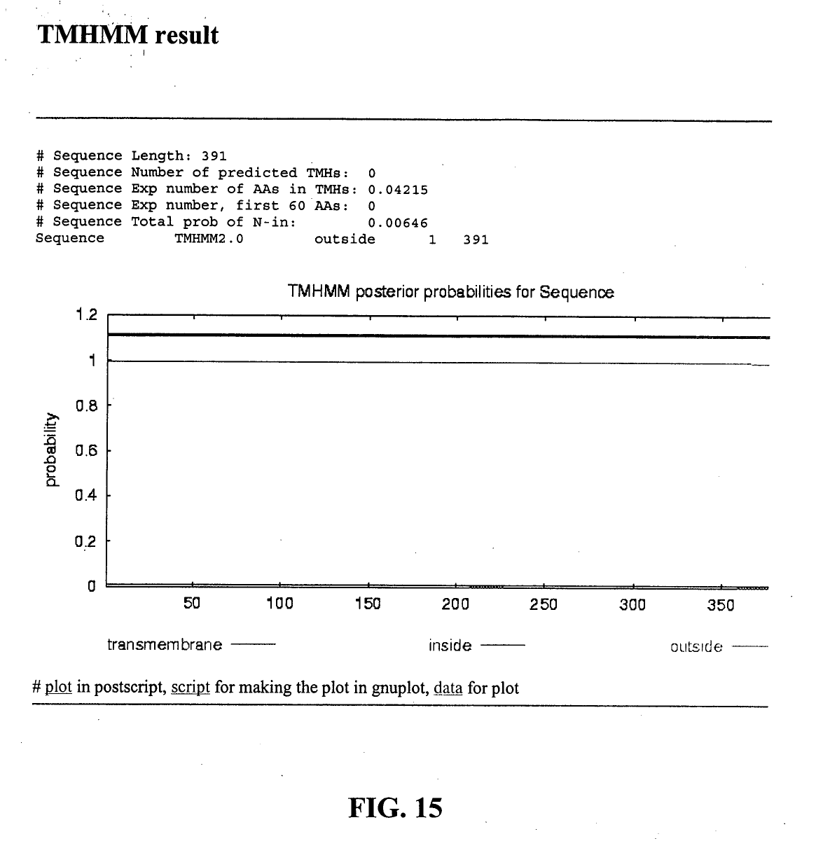

CSNK2A1 (casein kinase 2, alpha 1 polypeptide): CSNK2A1 is a ser/thr protein kinase. It is very similar to murine Csnk2a1, which is an oncogene when expressed inappropriately. CSNK2A1 phosphorylates acidic protein such as casein. It has a tetrameric a(2)/b(2) structure. The alpha subunit of molecular weight 40,000 possesses catalytic activity, whereas the beta subunit, molecular weight 25,000, is autophosphorylated in vitro. Phosphorylation of the human p53 protein at ser392 is responsive to ultraviolet (UV) but not gamma irradiation. identified and purified a mammalian UV-activated protein kinase complex that phosphorylates ser392 in vitro. This kinase complex contains CK2 and the chromatin transcriptional elongation factor FACT, a heterodimer of SPT16 and SSRP1. In vitro studies showed that FACT alters the specificity of CK2 in the complex such that it selectively phosphorylates p53 over other substrates, including casein. In addition, phosphorylation by the kinase complex was found to enhance p53 activity. These results provided a potential mechanism for p53 activation by UV irradiation. The TMHMM profile of CSNK2A1 is shown in FIG. 15.

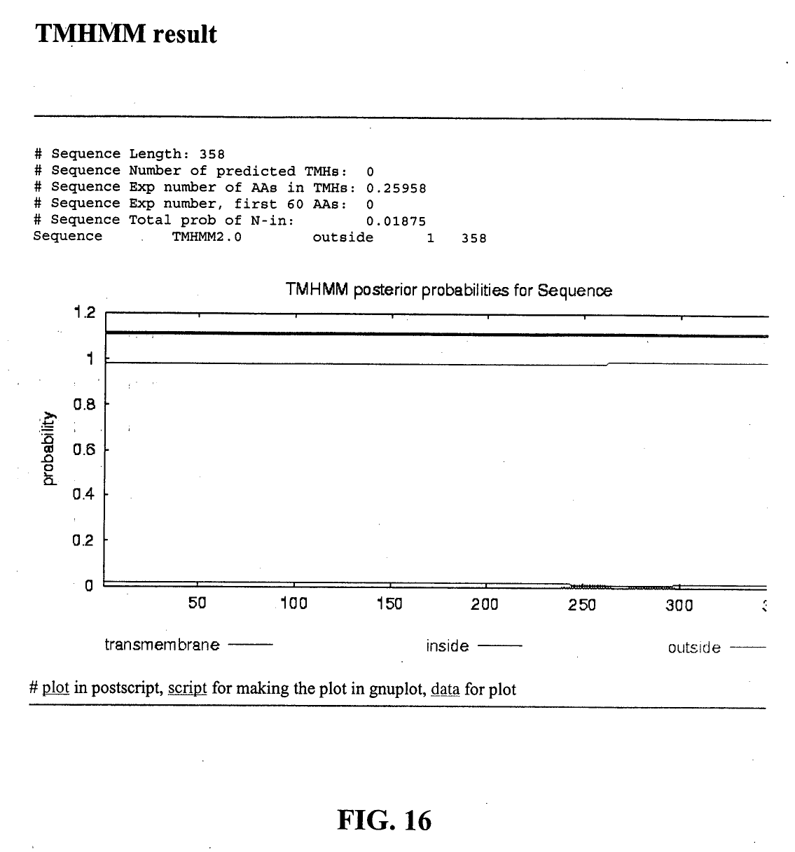

C20orf97 (protein kinase domains containing protein similar to phosphoprotein C8FW): The protein is phosphorylated as cells enter mitosis and dephosphorylated as cells exit mitosis (by similarity). C20orf97 belongs to the ser/thr family of protein kinases and CDC5/polo subfamily. It is involved in regulating M phase functions during the cell cycle and may also be part of the signaling network controlling cellular adhesion. C20orf97 is capable of phosphorylating CDC25c and casein in vitro. The TMHMM profile of C20orf97 is shown in FIG. 16.

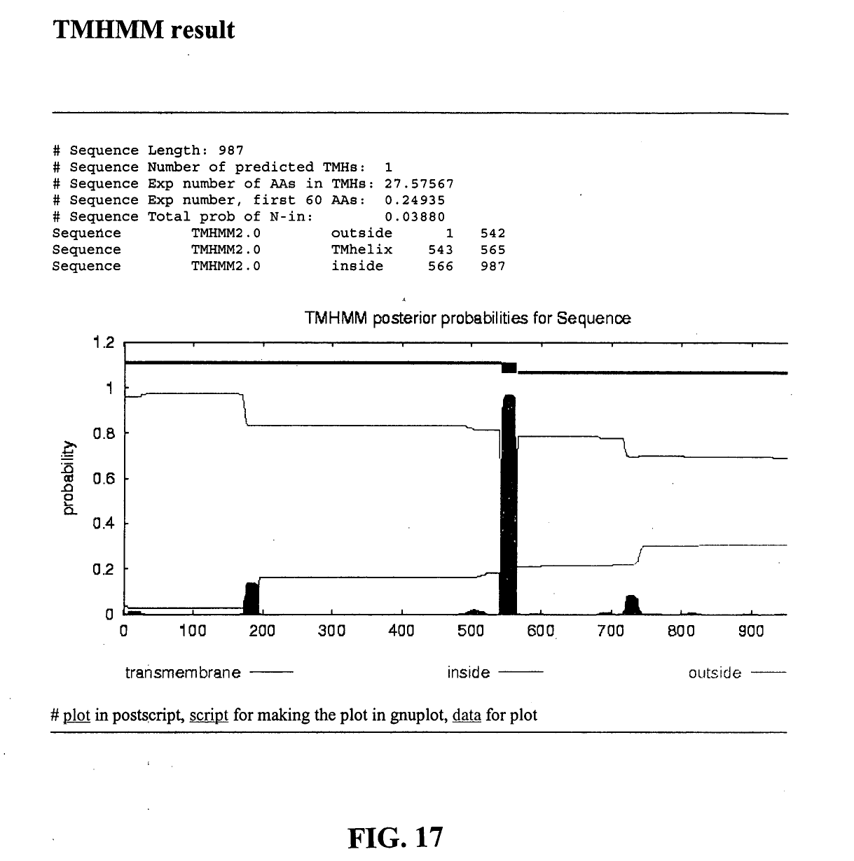

EPHB2 (Eph-related receptor tyrosine kinase B2): EPHB2 is one of the EPH receptors. The ligands of the EPH receptors are the ephrins. The EPH and EPH-related receptors comprise the largest subfamily of receptor protein-tyrosine kinases. They have been implicated in mediating developmental events, particularly in the nervous system. Northern blot analysis revealed that EPHB2 is expressed as transcripts of several sizes in a variety of human tissues, with the highest level of expression in the placenta. The related EPHB3 receptor was expressed in all of the adult tissues tested. The TMHMM profile of EPHB2 is shown in FIG. 17.

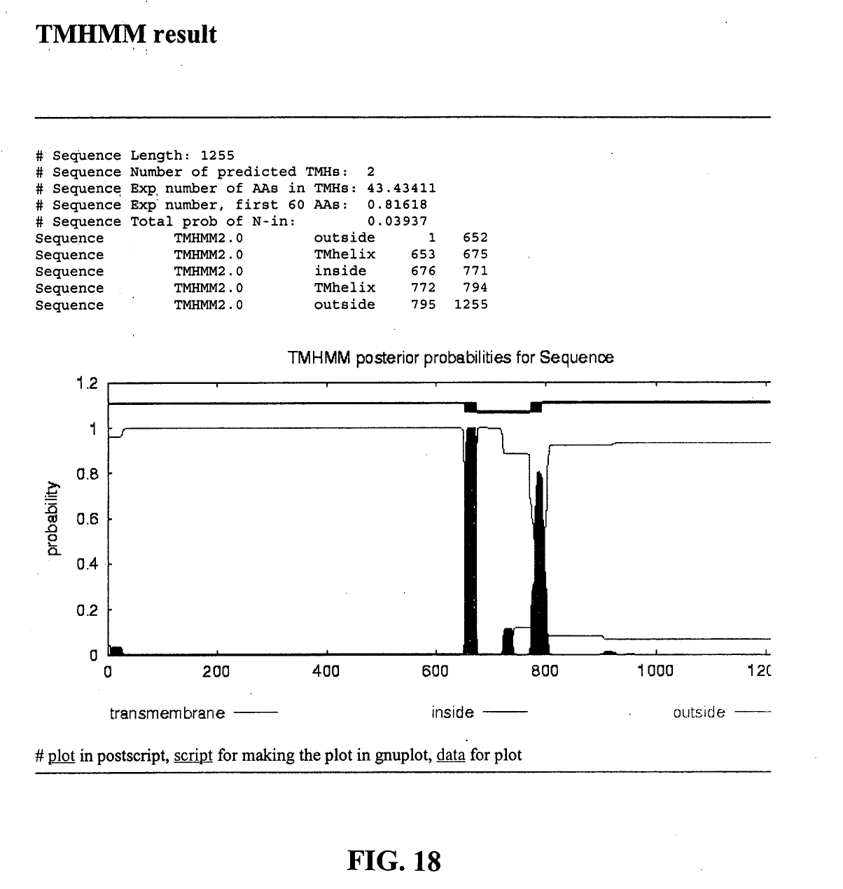

ERBB2/HER2/NEU (v-erb-b2 erythroblastic leukemia viral oncogene homolog 2, neuro/glioblastoma derived oncogene homolog (avian)): ERBB2 is a tyrosine kinase receptor and a component of IL-6 signaling through the MAP kinase pathway. ERBB2 is similar to the EGF receptor. Overexpression of ERBB2 confers Taxol resistance in breast cancer cells. Transfected MDA-MB-435 cells that overexpress HER2 transcriptionally upregulates CDKN1A which when associated with CDC2 would inhibit Taxol-mediated CDC2 activation, and delay cell entrance to G₂/M phase, and thereby inhibits Taxol-induced apoptosis. In CDKNIA anti sense-transfected MDA-MB-435 cells or in p21/MEF cells, ERBB2 was unable to inhibit Taxol-induced apoptosis. Therefore, CDKNIA participates in the regulation of a G₂/M checkpoint that contributes to resistance to Taxol-induced apoptosis in ERBB2-over-expressing breast cancer cells. The TMHMM profile of ERBB2 is shown in FIG. 18.

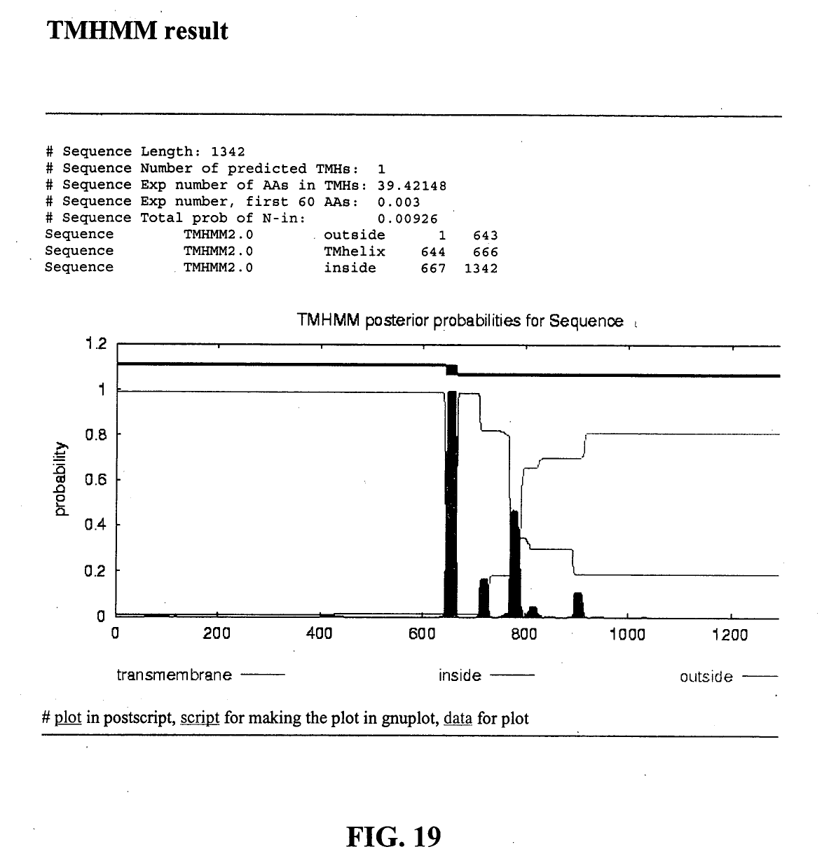

ERBB3/HER3(v-erbB2 erythroblastic leukemia viral oncogene homolog 3 (avian)): ERBB3 is a tyrosine kinase receptor that binds heregulin. Markedly elevated ERBB3 mRNA levels were demonstrated in certain human mammary tumor cell lines, suggesting that it may play a role in some human malignancies just as does EGFR. The TMHMM profile of ERBB3 is shown in FIG. 19.

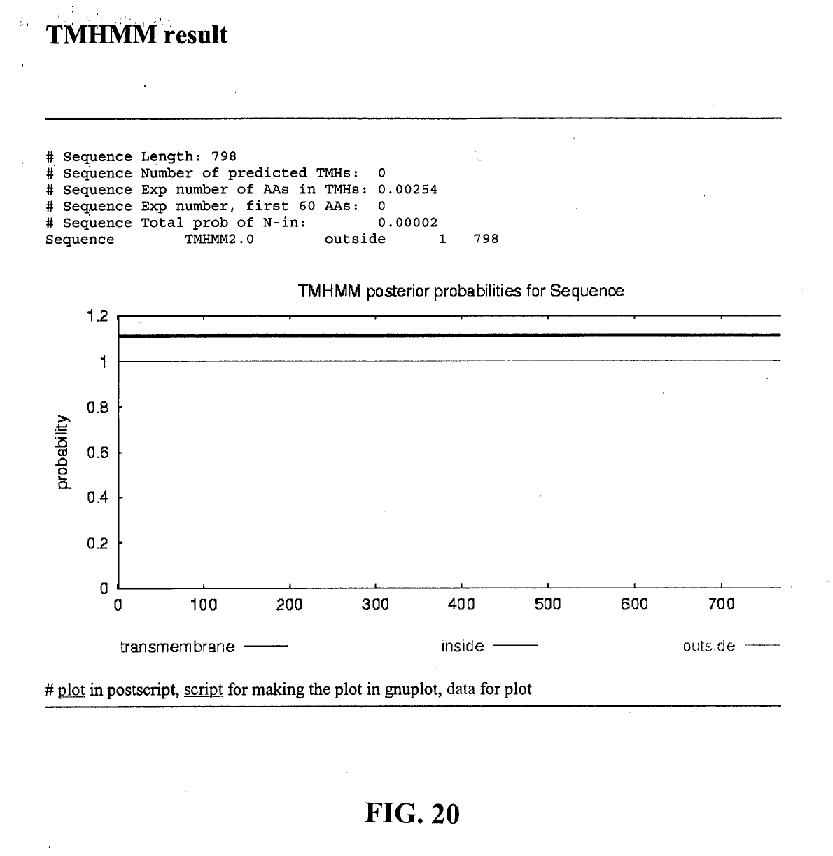

GSG2 (haspin): GSG2 is a predicted protein with tyrosine and ser/thr kinase domains. GSG2 may play a role in cell-cycle cessation and differentiation of haploid germ cells. In addition, GSG2 mRNA can be detected in diploid cell lines and tissues. GSG2-like proteins are identified in several major eukaryotic phyla-including yeasts, plants, flies, fish, and mammals-and an extended group in C elegans. The TMHMM profile of GSG2 is shown in FIG. 20.

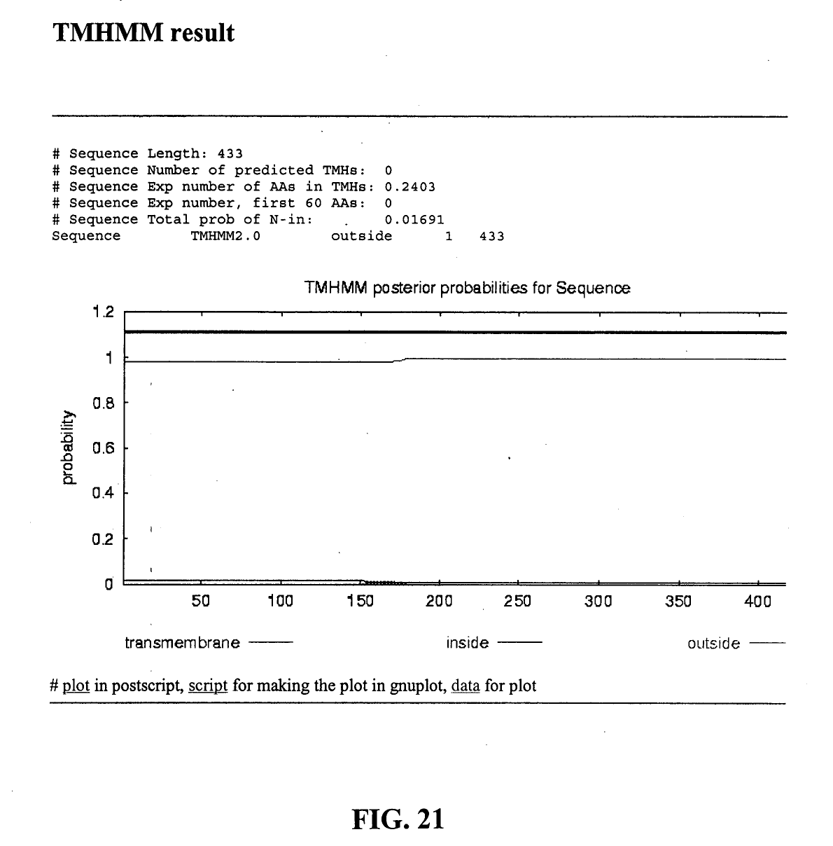

GSK3B (glycogen synthase kinase 3 beta): The intracellular distribution of GSK3B is dynamically regulated by signaling cascades, and apoptotic stimuli cause increased nuclear levels of GSK3B, which facilitates interactions with nuclear substrates. GSK3B is implicated in the hormonal control of several regulatory proteins including glycogen synthase, myb, and the transcription factor c-jun. GSK3B phosphorylates c-jun at sites proximal to its DNA-binding domain, reducing DNA-binding affinity. GSK3B is phosphorylated by AKT1 and ILK1. The ILK protein is a ser/thr protein kinase with 4 ankyrin-like repeats. ILK regulates integrinmediated signal transduction. GSK3B-deficient mice have been generated by targeted disruption. Although GSK3B +/− male and female mice were healthy and fertile, they did not give rise to live GSK3B −/− progeny. Embryonic lethality occurred between embryonic days 13.5 and 14.5 due to severe liver degeneration, a phenotype consistent with excessive tumor necrosis factor TNF toxicity, as observed in mice lacking genes involved in the activation of transcription factor NFKB. GSK3B-deficient embryos were rescued by inhibition of TNF using anti-TNF-alpha antibody. Fibroblasts from GSK3B-deficient embryos were hypersensitive to TNF-alpha and showed reduced NFKB function. Lithium treatment, which inhibits GSK3, sensitized wildtype fibroblasts to TNF and inhibited transactivation of NFKB. The early steps leading to NFKB activation were unaffected by the loss of GSK3B, indicating that NFKB is regulated by GSK3B at the level of the transcriptional complex. The TMHMM profile of GSK3B is shown in FIG. 21.

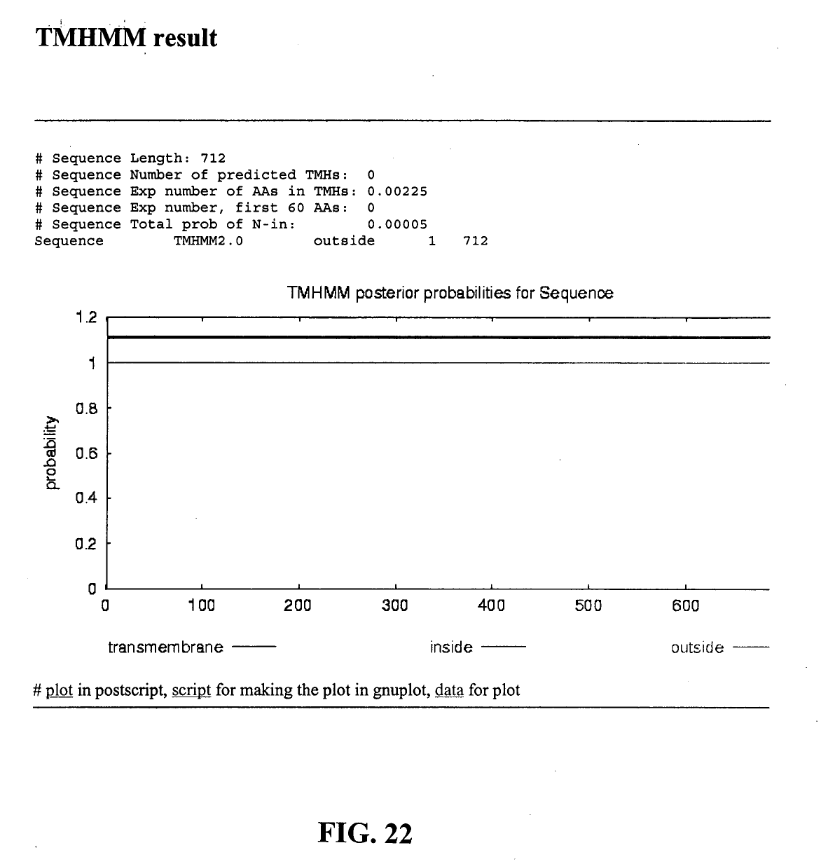

IRAK1 (interleukin-1 receptor-associated kinase 1): IRAK1 is one of the two putative ser/thr kinases that become associated with the interleukin-1 receptor (IL1R) upon stimulation. IRAK1 is partially responsible for IL1-induced upregulation of the transcription factor NF-kappa B. The TMHMM profile of IRAK1 is shown in FIG. 22.

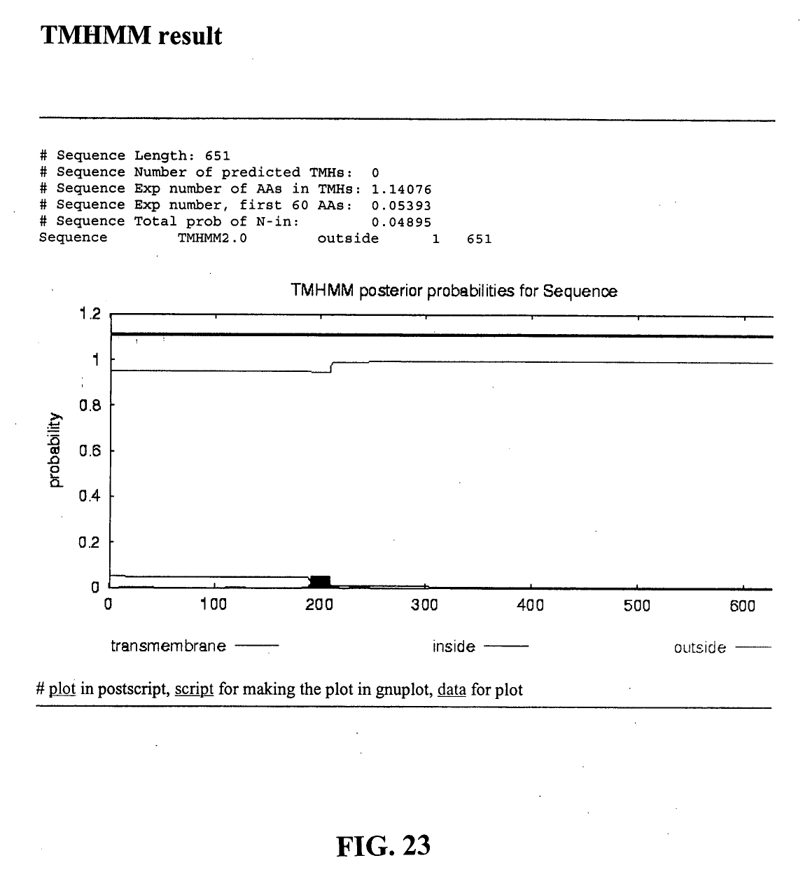

KIAA0175: This gene is the likely orthologue of the murine maternal embryonic leucine zipper kinase which is a member of the SnfI/AMPK kinase family. The mouse gene is known as MELK and encodes a protein with a catalytic domain and a leucine zipper motif. SnfI is a histone kinase that works in concert with the histone acetyltransferase Gen5 to regulate transcription. This gene product may play a role in signal transduction events in the egg and early embryo. The TMHMM profile of KIAA0175 is shown in FIG. 23.

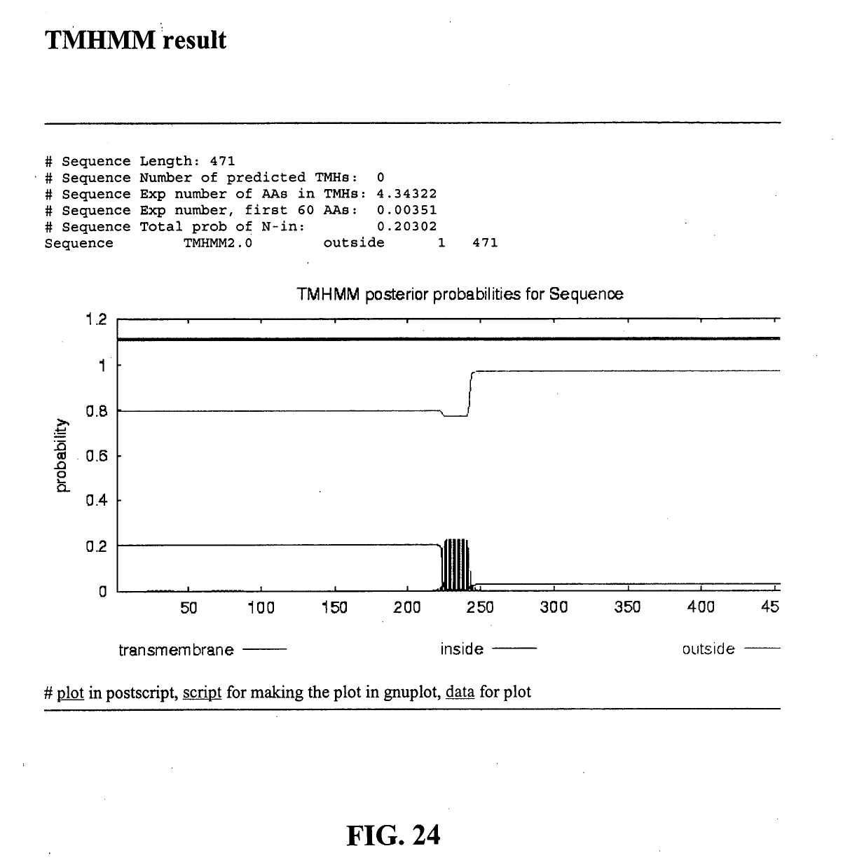

MAPKAPK5 (mitogen-activated protein kinase-activated protein kinase 5): MAPKAPK5 is a ser/thr kinase that phosphorylates HSP2 and may have a role in stress response. MAPKAPK5 is a 471-amino acid protein that shares 20 to 30% sequence identity with RSK, MNK, and MAPKAPK kinases. MAPKAPK5 contains the conserved protein kinase domains I through XI, which are characteristic of all protein kinases. The TMHMM profile of MAPKAPK5 is shown in FIG. 24.

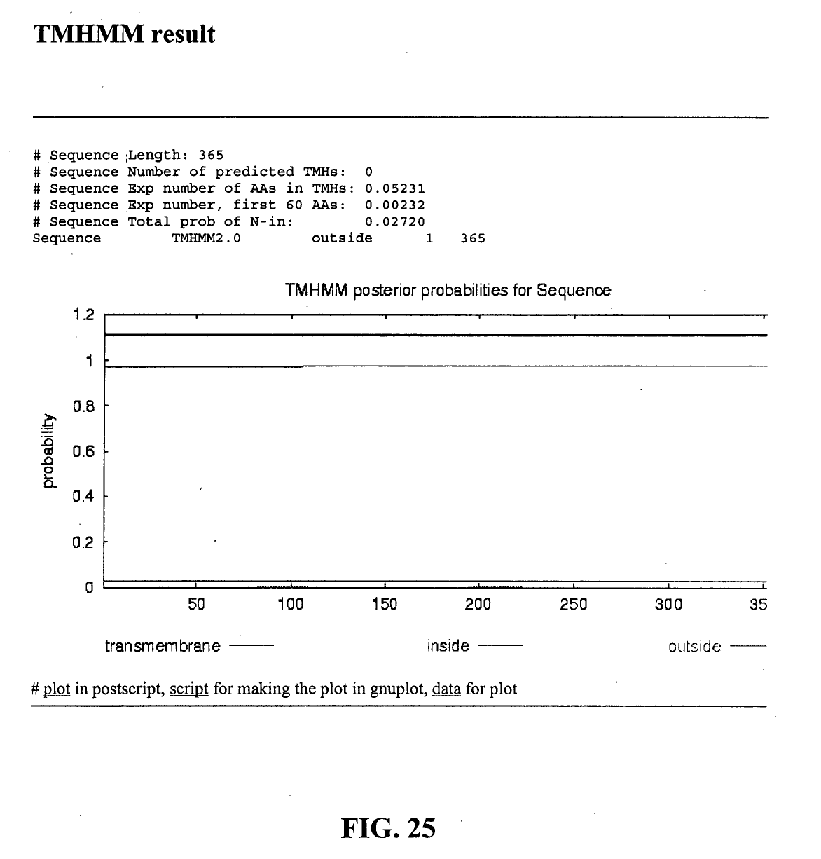

MAPK13 (mitogen-activated protein kinase 13): MAPK13 is a member of the MAP kinase family. MAP kinases act as an integration point for multiple biochemical signals, and are involved in a wide variety of cellular processes such as proliferation, differentiation, transcription regulation and development. MAPK13 is closely related to p38 MAP kinase, both of which can be activated by proinflammatory cytokines and cellular stress. MAP kinase kinases 3, and 6 can phosphorylate and activate MAPK13. Transcription factor ATF2, and microtubule dynamics regulator stathmin have been shown to be the substrates of this kinase. MAPK13 is activated by stress and proinflammatory cytokines and is phosphorylated by MKK6 (PRKMK6). Mitogen-activated protein kinase (MAPK) cascades represent one of the major signal systems used by eukaryotic cells to transduce extracellular signals into cellular responses. The stress-activated protein kinases (SAPKS) are MAPKs that are activated by chemical and environmental stresses as well as by proinflammatory cytokines. The TMHMM profile of MAPK1 3 is shown in FIG. 25.

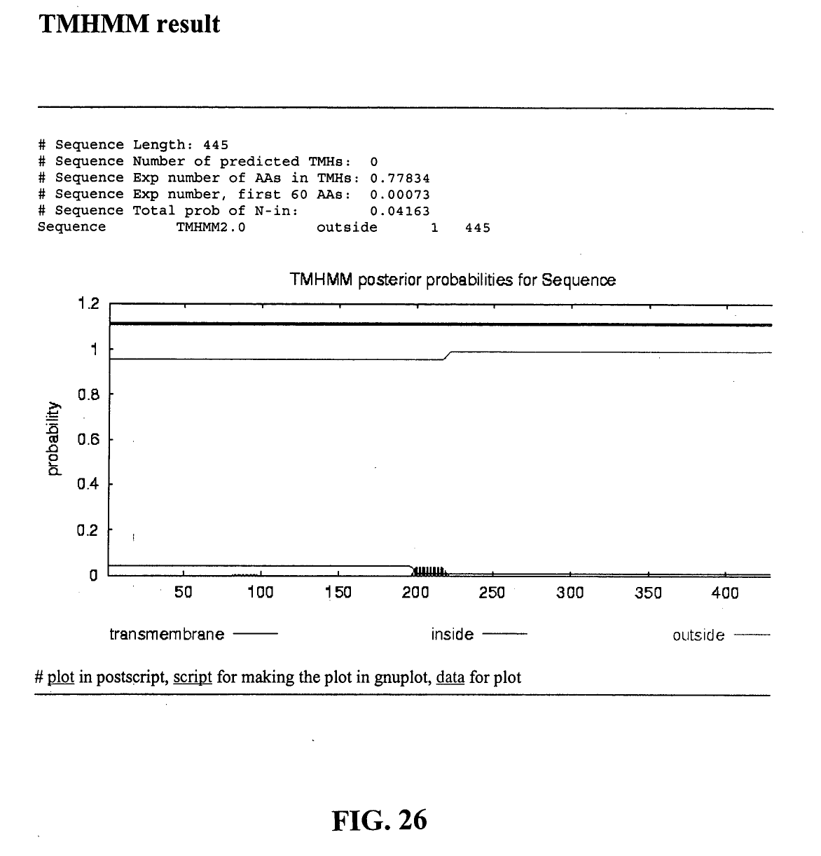

NEK2/NIMA (never in mitosis gene A) related kinase 2): NIMA was first characterized in Aspergillus nidulans as required for entry into mitosis. Cells with a NIMA mutation arrest in G2 while over-expression induces mitosis. Using Western blots of extracts of synchronized HeLa cells, it has been shown that the NEK2 protein was almost undetected during GI, but accumulated progressively throughout S phase reaching maximal levels in late G2. NEK2 localized to the centrosome throughout the cell cycle. Over-expression of active NEK2 induces a splitting of the centrosomes, which probably results from the phosphorylation of the centrosomal proteins by NEK2. NEK2 may play a role in the regulation of centrosome separation. The TMHMM profile of NEK2 is shown in FIG. 26.

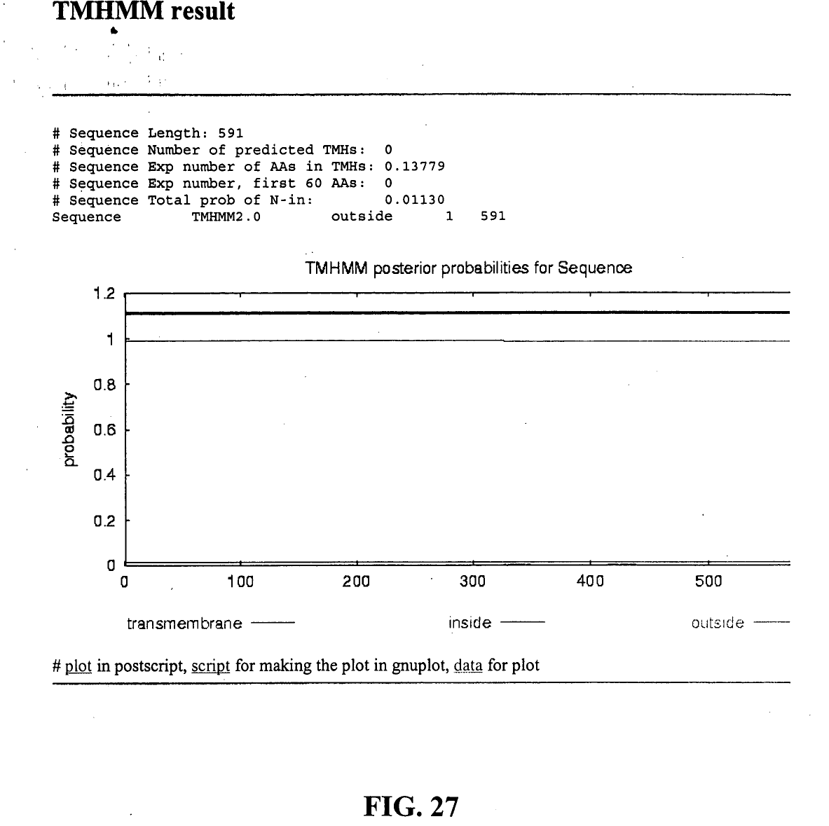

PAK4 (p21(CDKNIA)-activated kinase 4): PAK proteins are critical effectors that link Rho GTPases to cytoskeleton reorganization and nuclear signaling. PAK proteins, a family of ser/thr p21-activating kinases, include PAK1, PAK2, PAK3 and PAK4. PAK proteins serve as targets for the small GTP binding proteins CDC42 and Rae and have been implicated in a wide range of biological activities. PAK4 interacts specifically with the GTP-bound form of CDC42Hs and weakly activates the JNK family of MAP kinases. PAK4 is a mediator of filopodia formation and may play a role in the reorganization of the actin cytoskeleton. The TMHMM profile of PAK4 is shown in FIG. 27.

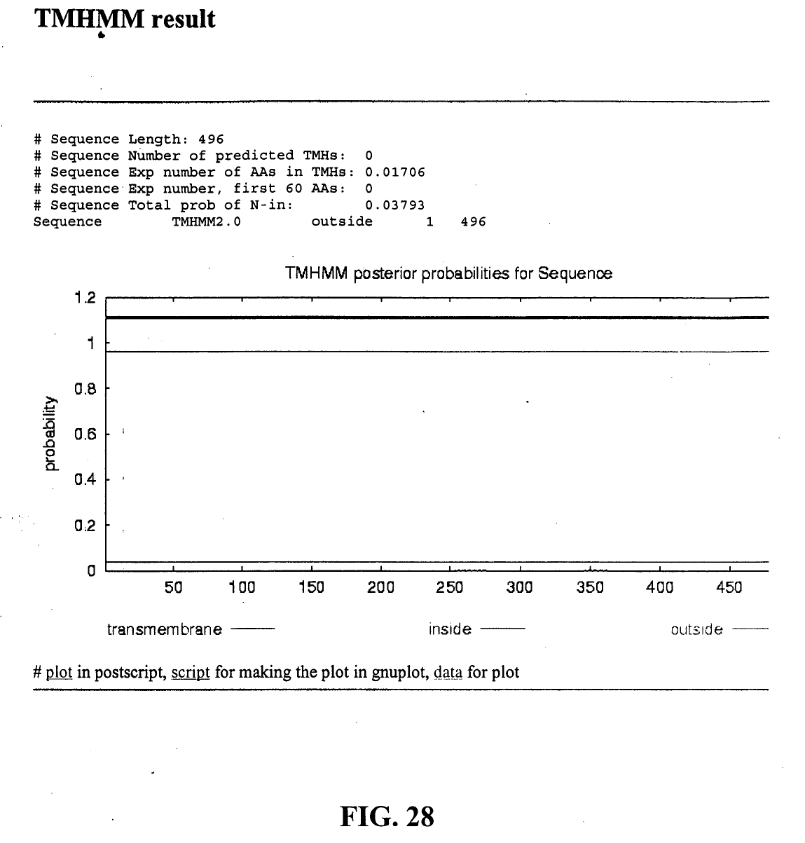

PCTK1/PCTAIRE protein kinase 1: PCTK1 belongs to the PCTAIRE protein kinases subfamily of CDC2 kinases (it is also named PCTAIRE protein kinase 1 for the presence of a cysteine-for-serine substitution in the conserved PSTAIRE amino acid motif found in prototypic CDC2 kinases). Three members of this kinase subfamily, PCTAME1-3, have been identified in humans. This ser/thr kinase may play a role in signal transduction cascades in terminally differentiated cells. The PCTK1 gene is thought to escape X inactivation. There are three alternatively spliced transcript variants described for this gene. PCTK1 is ubiquitously expressed with the highest levels detected in the brain and testis. The TMHMM profile of PCTK1 is shown in FIG. 28.

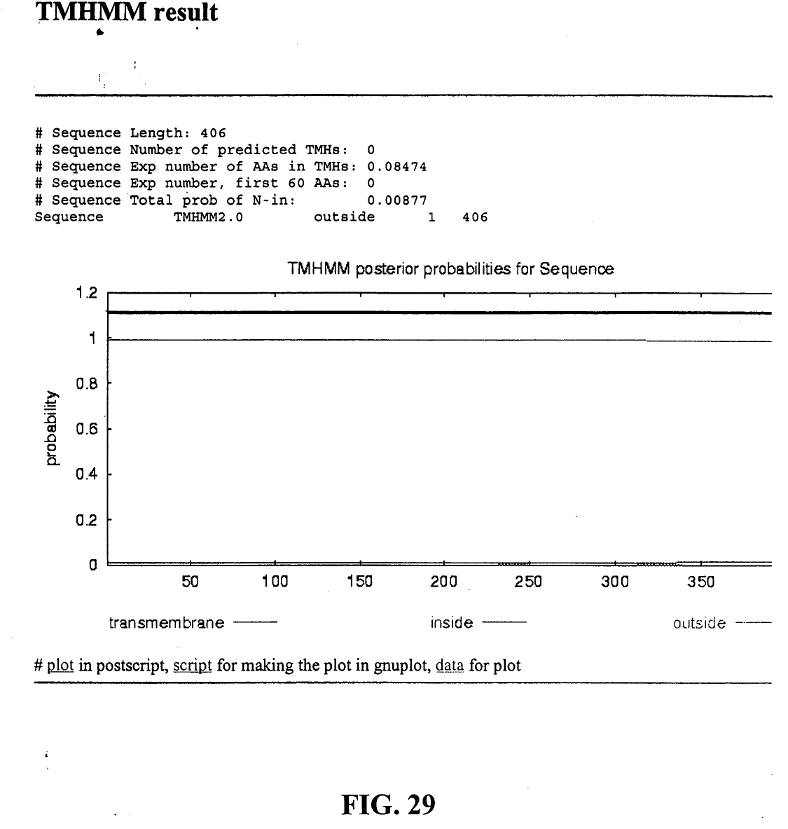

PDK3 (pyruvate dehydrogenase kinase, isoenzyme 3): PDK3 phosphorylates the El alpha subunit of the pyruvate dehydrogenase complex and regulates glucose metabolism. The TMHMM profile of PDK3 is shown in FIG. 29.

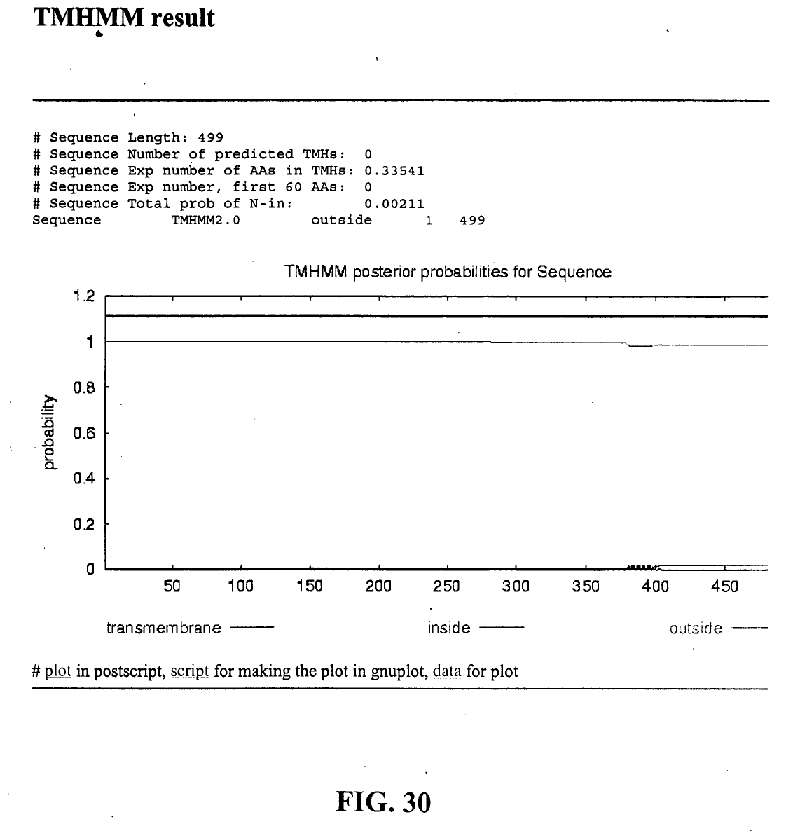

PKMYTI (membrane-associated tyrosine- and threonine-specific cdc2-inhibitory kinase): PKMYTI inhibits the activity of cyclin-bound CDC2 by phosphorylating the protein at residue thr14. Entry into mitosis requires the activity of CDC2 kinase coupled with cyclin B. Phosphorylation of the CDC2 protein on residues thr14 and tyr15 is inhibitory to CDC2 activity. PKMYT1 is, in effect, an inhibitor of mitosis. The TMHMM profile of PKMYT1 is shown in FIG. 30.

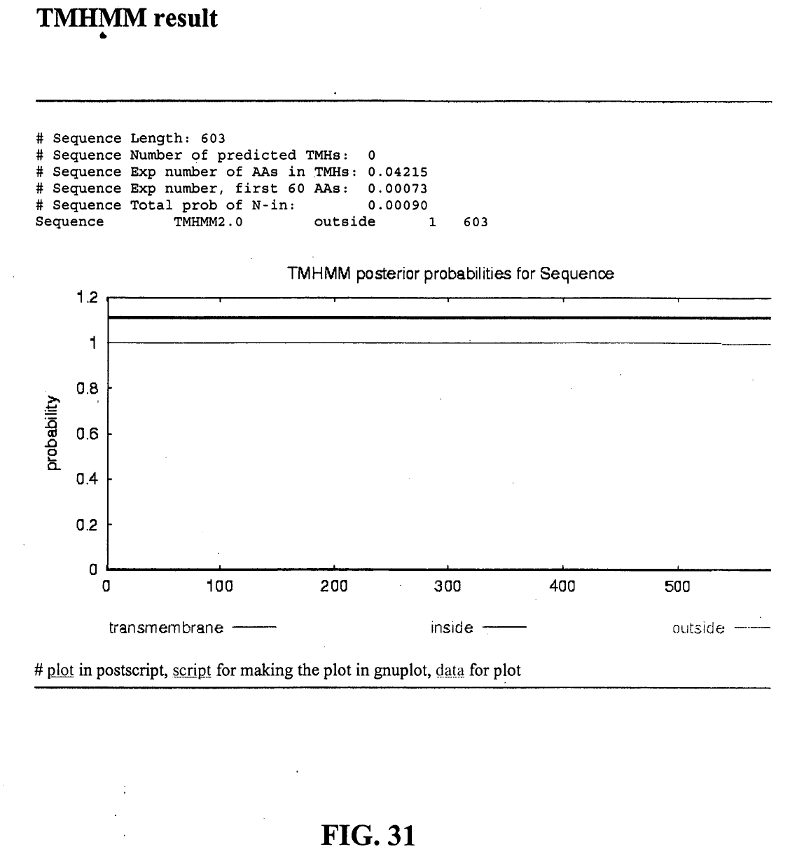

PLK (Polo (Drosophia)-like kinase): PLK is a ser/thr kinase that is active in chromosomal segregation. PLK has been shown not to be expressed in any adult human tissues except placenta. Among cultured cell lines, PLK mRNA was detected in all growing cells. PLK localizes to the mitotic spindle and is thought to be involved in the promotion or progression of cancers. Cells transformed with PLK grew in soft agar and produced tumors in nude mice. PLK may be involved in targeting MPF (mitosis promoting factor) to the nucleus during prophase. The TMHMM profile of PLK is shown in FIG. 31.

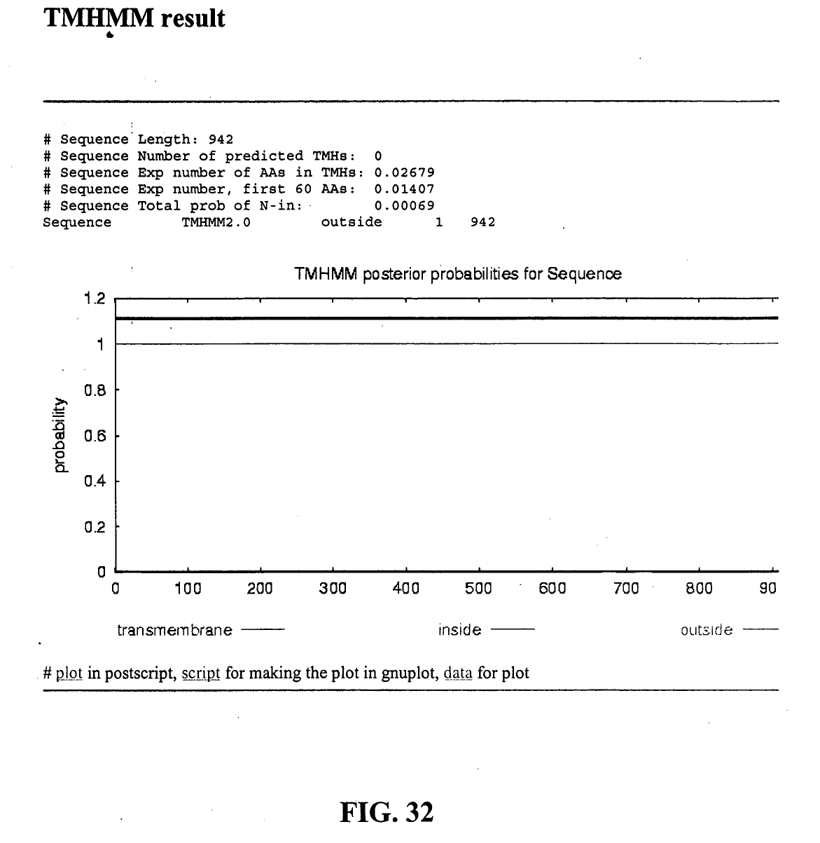

PRKCL1 (protein kinase C-like 1): PRKCL1 phosphorylates ribosomal protein s6, and mediates GTPase rho-dependent intracellular signaling. The putative 942-amino acid protein has leucine zipper-like sequences at its amino terminus and contains a domain with strong similarity to that of the protein kinase C family. PRKCL1 is ubiquitously expressed in human tissues. Antisera detected a 120-kD recombinantly expressed protein on Western blots. The protein showed intrinsic protein kinase activity that was abolished by a mutation in the predicted ATP binding site. The TMHMM profile of PRKCL1 is shown in FIG. 32.

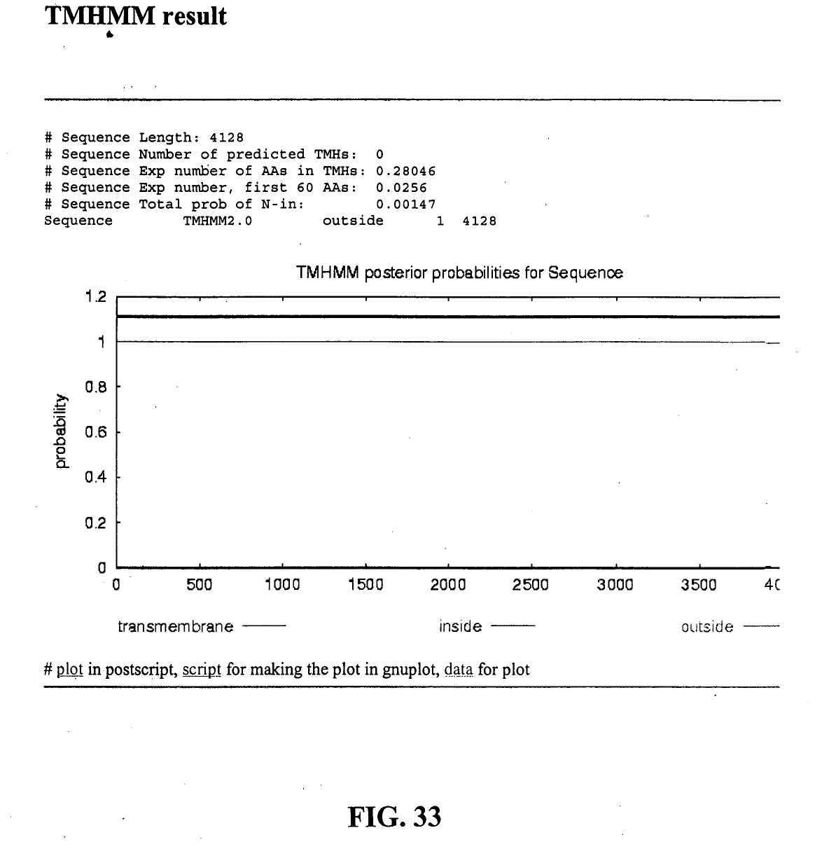

PRKDC (protein kinase, DNA-activated, catalytic polypeptide): PRKDC is the catalytic subunit of DNA-activated (DNA dependent) protein kinase. It has a role in DNA double strand break repair and recombination and has similarity to PI3Ks. PRKDC is a nuclear protein ser/thr kinase that is present in a variety of eukaryotic species. This kinase is not required for p53-dependent response to DNA damage. The hydrophobicity profile of PRKDC is shown in FIG. 33.

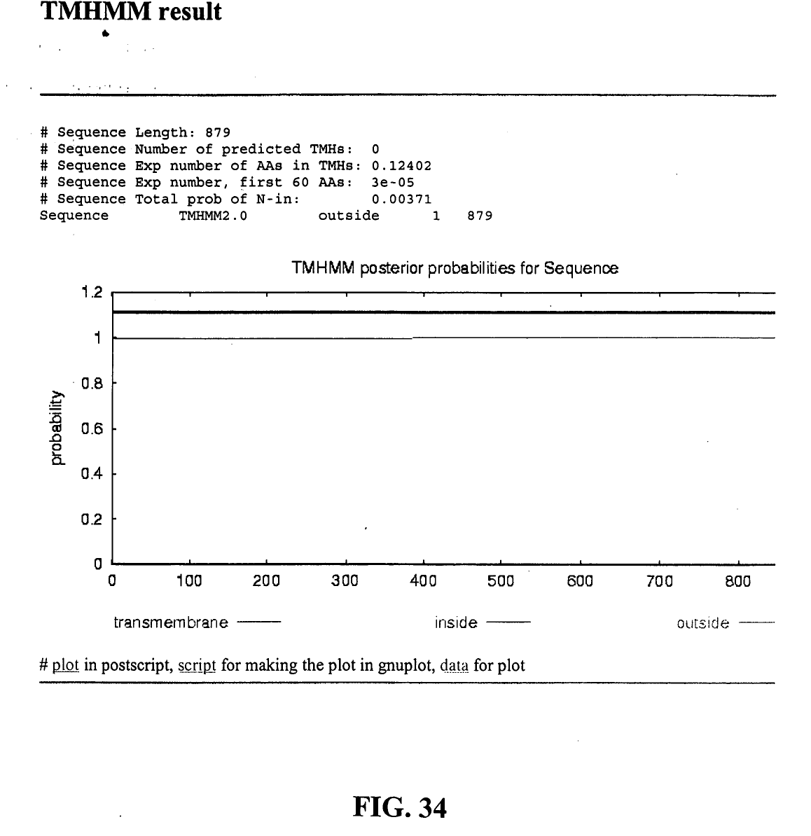

PTK2 (protein tyrosine kinase 2): PTK2 is a putative homolog of chicken focal adhesion associated kinase (FAK). Activation of PTK2 may be an important early step in cell growth and intracellular signal transduction pathways triggered in response to several neural peptides and/or to cell interactions with the extracellular matrix. Activation of focal adhesion kinases (FAK) may be an early step in intracellular signal transduction pathways. This tyrosine-phosphorylation is triggered by integrin interactions with various extracellular matrix adhesive molecules and by neuropeptide growth factors. PTK2 may also play a role in oncogenic transformation resulting in increased kinase activity. The TMHMM profile of PTK2 is shown in FIG. 34.

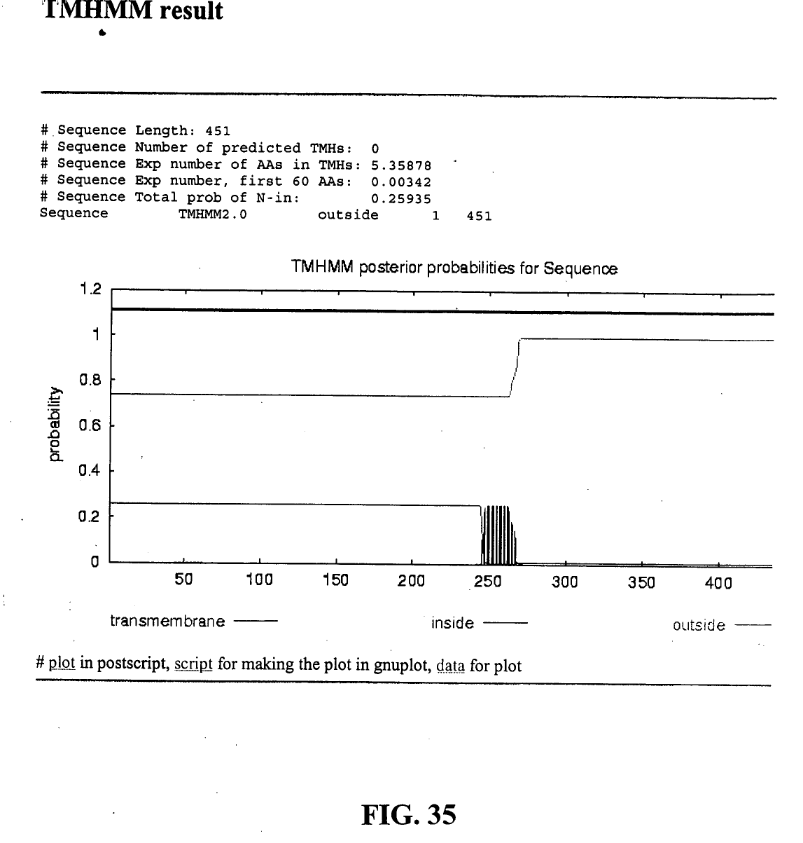

PTK6 (PTK6 protein tyrosine kinase 6): PTK6 is a non-receptor protein tyrosine kinase and is involved in sensitization of mammary epithelial cells to epidermal growth factor (EGF). PTK6 is capable of tyrosine autophosphorylation. Overexpression of PTK6 in mammary epithelial cells led to sensitization of cells to epidermal growth factor (EGF; and resulted in a partially transformed phenotype. Coimmunoprecipitation of BRK and the EGF receptor has been reported. The TMHMM profile of PTK6 is shown in FIG. 35.

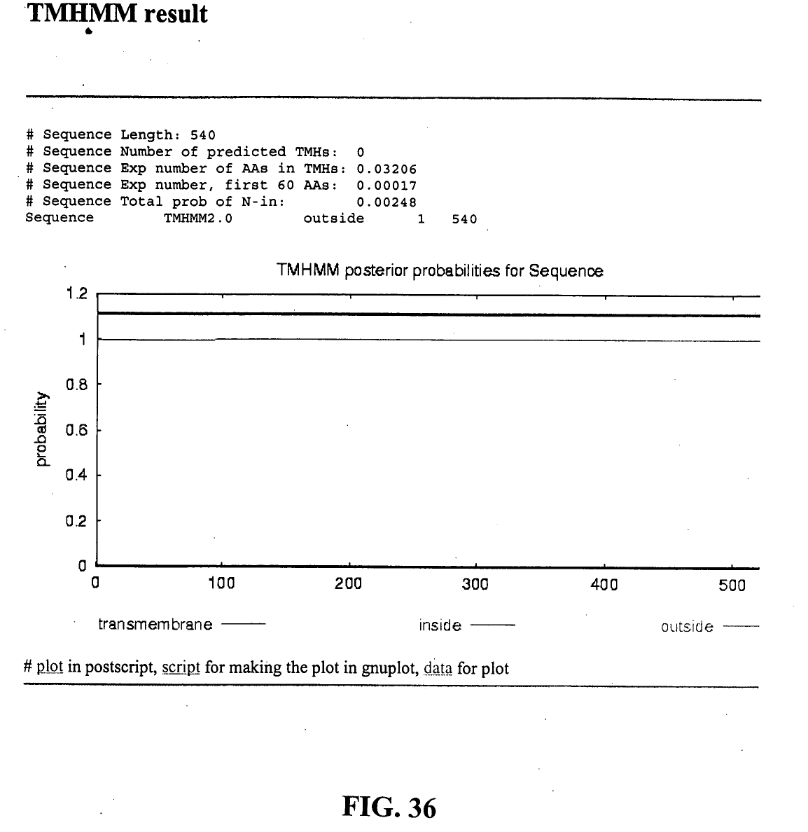

RIPK2 (receptor-interacting ser/thr kinase 2): RIPK2 interacts with CD40 or the tumor necrosis factor receptor; and has a C-terminal domain for caspase recruitment and activation. RIPK2 is a death domain-containing protein kinase that interacts with the death domain of FAS, but does not appear to mediate FAS-initiated apoptosis. The 540-amino acid protein contains an N-terminal ser/thr kinase catalytic domain and a C-terminal caspase activation and recruitment domain (CARD). RIPK2-knockout mice are viable and fertile. However, the subclass-specific IgG responses are lower in RIPK2-deficient mice. T-cell responses, specifically Th1 differentiation and cytokine production, are more severely affected. IFNγ production in response to T-cell receptor activation plus IL12 and/or IL18 stimulation was also reduced in the RIPK2-deficient mice, possibly through defective Stat4 activation. NK cells in the RIPK2-deficient mice were also unable to produce IFNγ in response to IL12 and/or IL18. The TMHMM profile of RIPK2 is shown in FIG. 36.

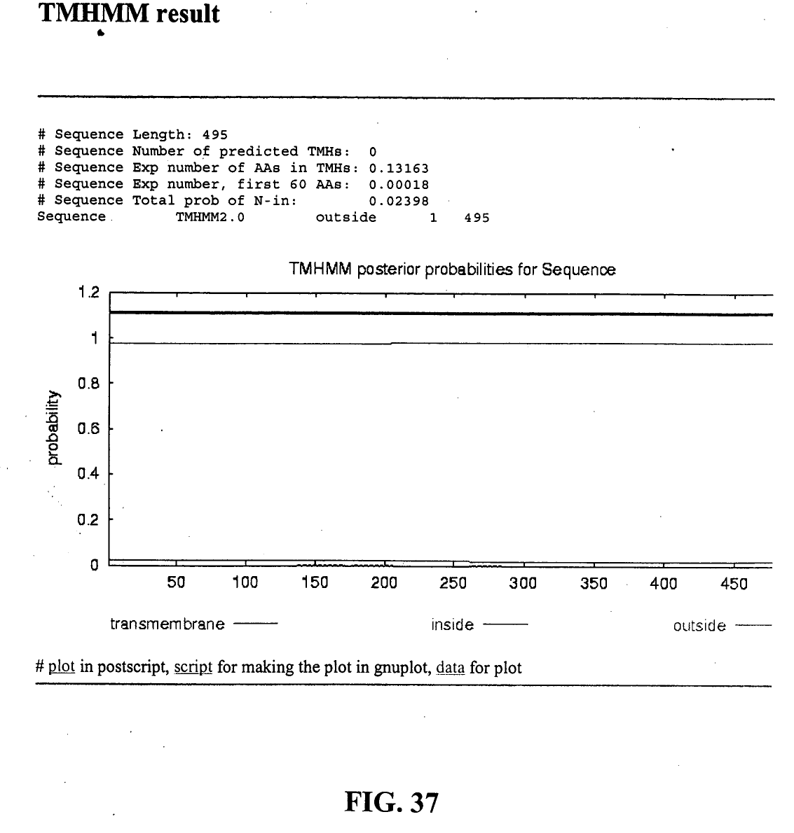

RPS6 KB2/P70S6 KB (ribosomal protein S6 kinase, 70 kD, polypeptide 2): RPS6 KB2 phosphorylates specifically ribosomal protein s6. The enzyme is activated by ser/thr phosphorylation and protein kinase C, and is inactivated by type 2a phosphatase. RPS6 KB2 has both tyrosine and serine/threonine catalytic domains. It is part of mTOR signal transduction pathway. The TMHMM profile of P70S6 KB is shown in FIG. 37.

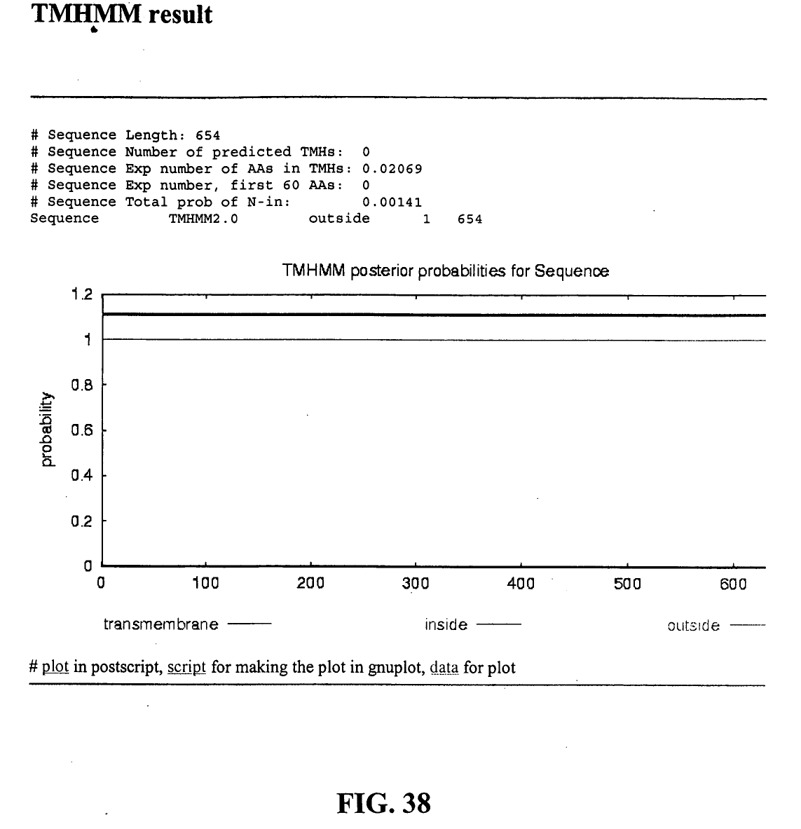

SRPK1 (SFRS protein kinase 1): This gene encodes a ser/arg protein kinase specific for the SR (serine/arginine-rich domain) family of splicing factors. The protein localizes to the nucleus and the cytoplasm. It is thought to play a role in regulation of both constitutive and alternative splicing by regulating intracellular localization of splicing factors. A second alternatively spliced transcript variant for this gene has been described, but its full length nature has not been determined. Inactivation of SRPK1 induces cisplatin resistance in a human ovarian carcinoma cell line. The TMHMM profile of SRPK1 is shown in FIG. 38.

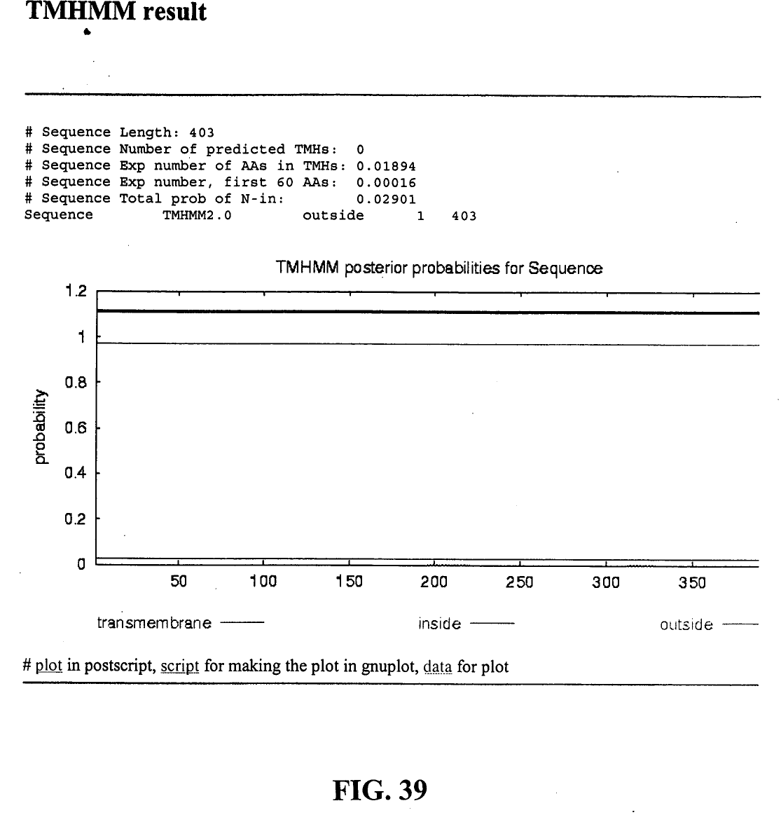

STK6/aurora/IPL1-like (ser/thr kinase 6): STK6 is most highly expressed during mitosis. It has high homology with Aurora and Ip11 kinases. Mutations in yeast STK6 are known to cause abnormal spindle formation and missegregation of chromosomes. Northern and Western blotting analyses revealed a high level of STK6 expression in testis and proliferating culture cells such as HeLa cells. The endogenous levels of STK6 protein and protein kinase activity were tightly regulated during cell cycle progression in HeLa cells. The protein was upregulated during G2/M and rapidly reduced after mitosis. Immunofluorescence studies revealed specific localization of STK6 protein to the spindle pole region during mitosis. The TMHMM profile of STK6 is shown in FIG. 39.

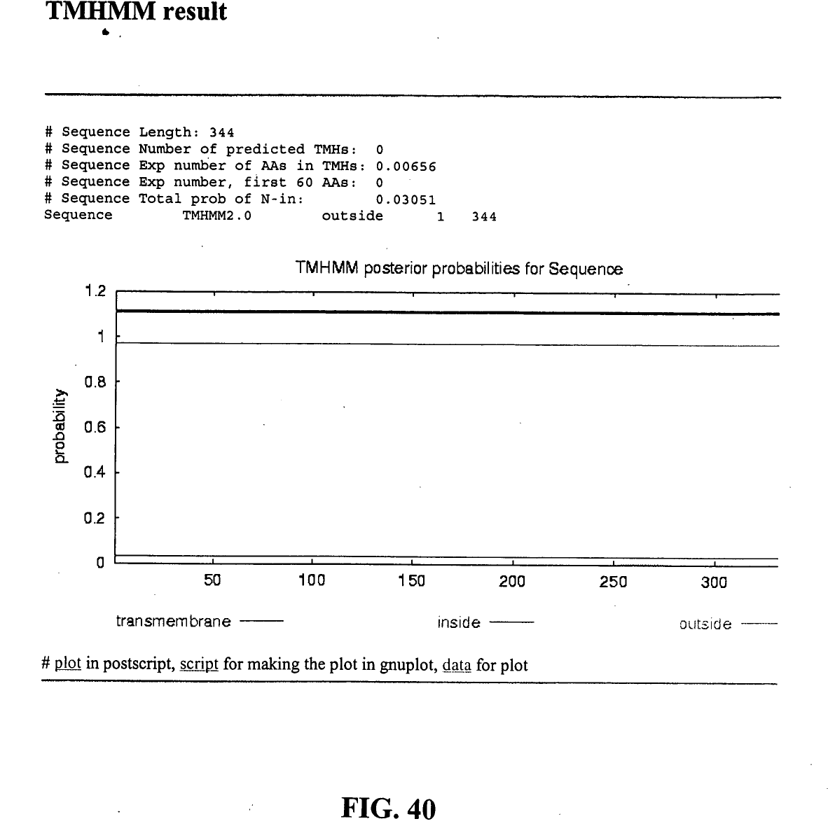

STK12/AURORA-RELATED KINASE 2/ARK2 (Ser/thr kinase 12): Drosophila ‘aurora’ and S. cerevisiae Ip1 1 ser/thr protein kinases (STKs) are involved in mitotic events such as centrosome separation and chromosome segregation. Human STK12 is a 344-amino acid protein containing kinase domains that share high homology with the catalytic domains of other STKs. Cell cycle and Northern blot analyses showed that STK12 is expressed in the S phase and persistently thereafter. Northern blot analysis detected strong expression of a 1.5-kb STK12 transcript in thymus, with weaker expression in small intestine, testis, colon, spleen, and brain. The TMHMM profile of STK12 is shown in FIG. 40.

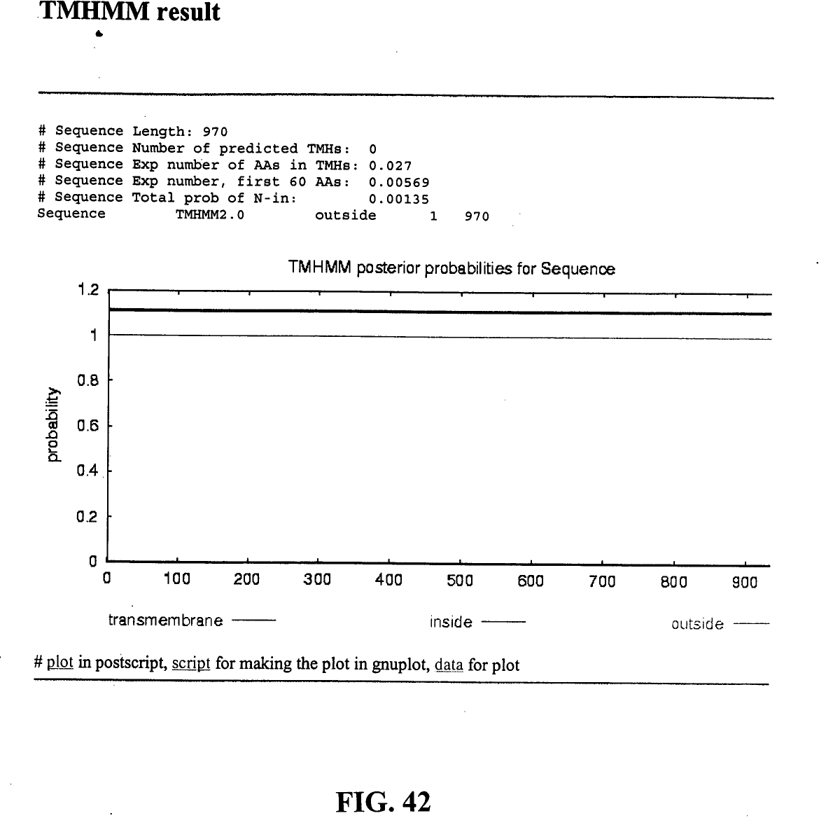

STK18 (ser/thr kinase 18): Chromosomal segregation during mitosis and meiosis is regulated by kinases and phosphatases. The cDNA encoding STK18 was isolated by screening embryonic tissue using degenerate PCR primers corresponding to conserved amino acid motifs within the catalytic domain of protein kinases, followed by screening a squamous cell carcinoma cDNA library. The predicted 970-amino acid STK18 protein shares significant homology with other STKs, particularly to those related to Drosophila ‘polo’, all of which have an N-terminal kinase domain. Because STK18 is homologous to the murine Sak gene, it is also named SAK. Northern blot analysis detected abundant expression of a 4.0-kb STK18 transcript in testis and thymus but not in other tissues or tumors. The TMHMM profile of STK18 is shown in FIG. 42.

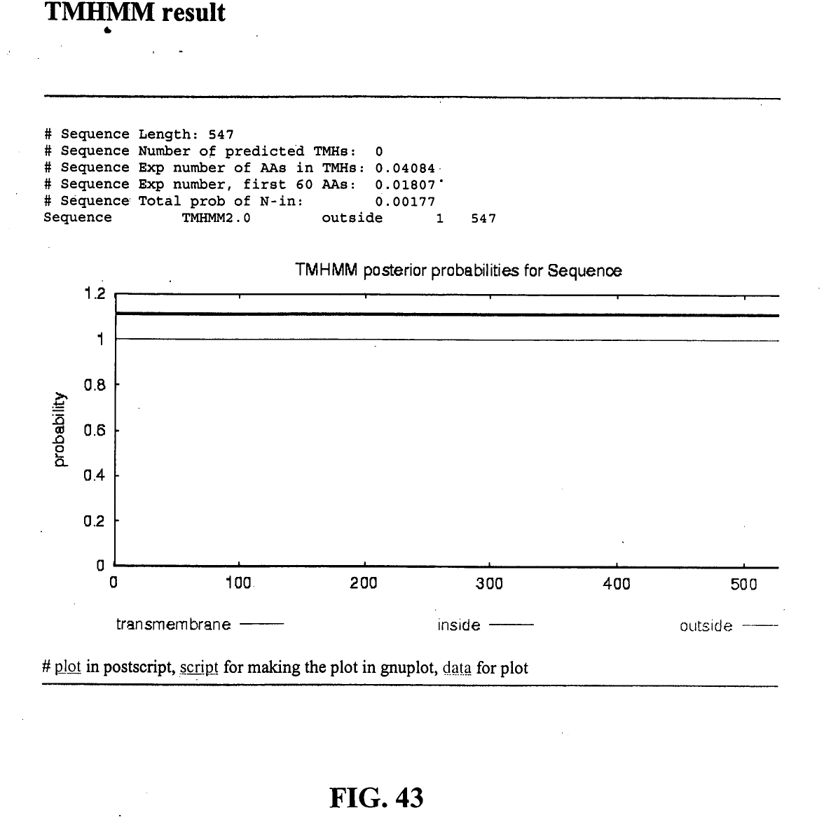

STK39/SPAK/Ste-20 related kinase: Human STK39 is very similar to rat SPAK. SPAK modulates p38 MAP kinase activity and exhibits increased expression in androgen-treated LNCaP cells. R1881-induced SPAK expression was completely abrogated by the antiandrogen casodex and by actinomycin D indicating that androgen induction of SPAK requires the androgen receptor and transcription. Cycloheximide caused a partial inhibition of R1881-induced SPAK expression which suggests that androgen induction of SPAK expression may require synthesis of additional proteins. Northern blot and ribonuclease protection assays demonstrated that SPAK is expressed at high levels in normal human testes and prostate, as well as in a number of breast and prostate cancer cell lines. The TMHMM profile of STK39 is shown in FIG. 43.

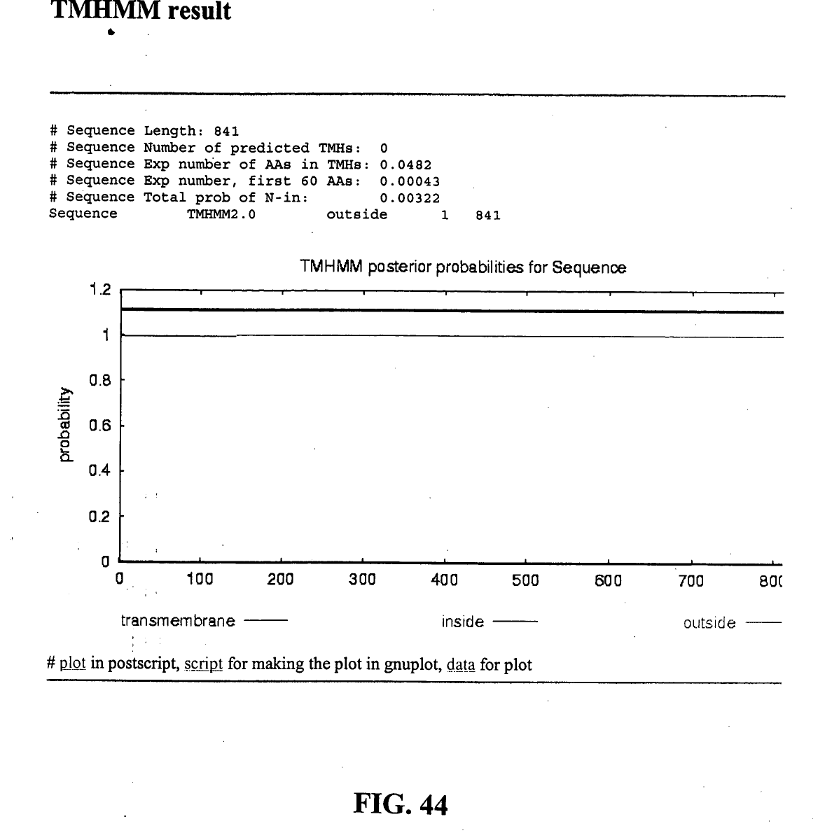

TTK protein kinase: This is a dual specific ser/thr and tyrosine kinase. It functions as a kinetochore-associated kinase whose activity is necessary to establish and maintain the mitotic checkpoint. The TMHMM profile of TTK is shown in FIG. 44.

Various aspects of the invention are described in further detail in the following sections and subsections. The use of sections and subsections is not meant to limit the invention; these section and subsections may apply to any aspect of the invention. As used herein, the term “or” means “and/or” unless otherwise specified.

Cancer-Related Protein Kinases (CPKs) and Cancer-Related Protein Kinase Genes (CPKGs)

1. CPKGs and Cancer

Table 1 provides CPKGs that are expressed at abnormally increased levels in human cancer tissues. These protein kinase genes may be a component in the disease mechanism and can be used as markers for diagnosing and monitoring cancer. Furthermore, CPKGs and CPKG products (CPKPNs and CPKPPs) may become novel therapeutic targets for the treatment and prevention of cancer.

Kinase proteins are a major target for drug action and development. A January 2002 survey of ongoing clinical trials in the USA revealed more than 100 clinical trials involving the modulation of kinases. Trials are ongoing in a wide variety of therapeutic indications including asthma, Parkinson's, inflammation, psoriasis, rheumatoid arthritis, spinal cord injuries, muscle conditions, osteoporosis, graft versus host disease, cardiovascular disorders, autoimmune disorders, retinal detachment, stroke, epilepsy, ischemia/reperfusion, breast cancer, ovarian cancer, glioblastoma, non-Hodgkin's lymphoma, colorectal cancer, non-small cell lung cancer, brain cancer, Kaposi's sarcoma, pancreatic cancer, liver cancer, and other tumors. Numerous kinds of modulators of kinase activity are currently in clinical trials including antisense molecules, antibodies, small molecules, and even gene therapy. The present invention advances the state of the art by providing new links of kinase proteins to the etiology of cancer.

Many therapeutic strategies are aimed at protein kinases since they are critical components in signal transduction pathways. Approaches for regulating kinase gene expression include specific antisense oligonucleotides for inhibiting post-transcriptional processing of the messenger RNA, naturally occurring products and their chemical derivatives to inhibit kinase activity and monoclonal antibodies to inhibit receptor linked kinases. In some cases, kinase inhibitors also allow other therapeutic agents additional time to become effective and act synergistically with current treatments.

The role of phosphorylation in transcriptional control, apoptosis, protein degradation, nuclear import and export, cytoskeletal regulation, and checkpoint signaling has been an important subject in pharmaceutical research. The accumulating knowledge about signaling networks and the proteins involved will be put to practical use in the development of potent and specific pharmacological modulators of phosphorylation-dependent signaling that can be used for therapeutic purposes. The rational structure-based design and development of highly specific kinase modulators is becoming routine and drugs that intercede in signaling pathways are becoming a major class of drug.

The kinases comprise the largest known protein group, a superfamily of enzymes with widely varied functions and specificities. They are usually named after their substrate, their regulatory molecules, or some aspect of a mutant phenotype. With regard to substrates, the protein kinases may be roughly divided into two groups; those that phosphorylate tyrosine residues (protein tyrosine kinases, PTK) and those that phosphorylate serine or threonine residues (ser/thr kinases, STK).

An important subfamily of the STK family is cyclic-AMP dependent protein kinases (PKA). Cyclic-AMP is an intracellular mediator of hormone action in all prokaryotic and animal cells that have been studied. Such hormone-induced cellular responses include thyroid hormone secretion, cortisol secretion, progesterone secretion, glycogen breakdown, bone resorption, and regulation of heart rate and force of heart muscle contraction. PKA is found in all animal cells and is thought to account for the effects of cyclic-AMP in most of these cells. Altered PKA expression is implicated in a variety of disorders and diseases including cancer, thyroid disorders, diabetes, atherosclerosis, and cardiovascular disease.

The mitogen-activated protein kinases (MAP) are also members of the STK family. MAP kinases also regulate intracellular signaling pathways. They mediate signal transduction from the cell surface to the nucleus via phosphorylation cascades. Several subgroups have been identified, and each manifests different substrate specificities and responds to distinct extracellular stimuli. MAP kinase signaling pathways are present in mammalian cells as well as in yeast. The extracellular stimuli that activate mammalian pathways include epidermal growth factor (EGF), ultraviolet light, hyperosmolar medium, heat shock, endotoxic lipopolysaccharide (LPS), and pro-inflammatory cytokines such as tumor necrosis factor (TNF) and interleukin-1 (IL-1).

EGF receptor is found in over half of breast tumors unresponsive to hormone. EGF is found in many tumors, and EGF may be required for tumor cell growth. Antibody to EGF blocked the growth of tumor xenografts in mice. An antisense oligonucleotide for amphiregulin inhibited growth of a pancreatic cancer cell line.

Cell proliferation and differentiation in normal cells are under the regulation and control of multiple MAP kinase cascades. Aberrant and deregulated functioning of MAP kinases can initiate and support carcinogenesis. Insulin and IGF-1 also activate a mitogenic MAP kinase pathway that may be important in acquired insulin resistance occurring in type 2 diabetes.

Many cancers become refractory to chemotherapy by developing a survival strategy involving the constitutive activation of the phosphatidylinositol 3-kinase-protein kinase B/Akt signaling cascade. This survival signaling pathway thus becomes an important target for the development of specific inhibitors that would block its function. PI-3 kinase/Akt signaling is equally important in diabetes. The pathway activated by RTKs subsequently regulates glycogen synthase 3 (GSK3) and glucose uptake. Since Akt has decreased activity in type 2 diabetes, it provides a therapeutic target.

Although some protein kinases have, to date, no known system of physiological regulation, many are activated or inactivated by autophosphorylation or phosphorylation by upstream protein kinases. The regulation of protein kinases also occurs transcriptionally, post-transcriptionally, and post-translationally. The mechanism of post-transcriptional regulation is alternative splicing of precursor mRNA. Protein kinase CβI and βII are two isoforms of a single PKCβ gene derived from differences in the splicing of the exon encoding the C-terminal 50-52 amino acids. Splicing can be regulated by a kinase cascade in response to peptide hormones such as insulin and IGF-1. PKCβI and βII have different specificities for phosphorylating members of the MAP kinase family, for glycogen synthase 3β, for nuclear transcription factors such as TLS/Fus, and for other nuclear kinases. By inhibiting the post-transcriptional alternative splicing of PKCβII mRNA, PKCβII-dependent processes are inhibited.

Protein kinase C isoforms have been implicated in cellular changes observed in the vascular complications of diabetes. Hyperglycemia is associated with increased levels of PKCα and β forms in renal glomeruli of diabetic rats. Oral administration of a PKCβ inhibitor prevented the increased mRNA expression of TGF-β1 and extracellular matrix component genes. Administration of the specific PKCβ inhibitor (LY333531) also normalized levels of cytokines, caldesmon and hemodynamics of retinal and renal blood flow. Over-expression of the PKCβ form in the myocardium resulted in cardiac hypertrophy and failure. The use of LY33353 1 to prevent adverse effects of cardiac PKCPβ over-expression in diabetic subjects is under investigation. The compound is also in Phase I/II clinical trials for diabetic retinopathy and diabetic macular edema indicating that it may be pharmacodynamically active.

PRK (proliferation-related kinase) is a serum/cytokine inducible STK that is involved in regulation of the cell cycle and cell proliferation in human megakaroytic cells. PRK is related to the polo (derived from human polo gene) family of STKs implicated in cell division. PRK is downregulated in lung tumor tissue and may be a proto-oncogene whose deregulated expression in normal tissue leads to oncogenic transformation. Altered MAP kinase expression is implicated in a variety of disease conditions including cancer, inflammation, immune disorders, and disorders affecting growth and development.

Protein kinase inhibitors provide much of our knowledge about in vivo regulation and coordination of kinase functions. A pseudosubstrate sequence within PKC acts to inhibit the kinase in the absence of its lipid activator. A PKC inhibitor such as chelerythrine acts on the catalytic domain to block substrate interaction, while calphostin acts on the regulatory domain to mimic the pseudosubstrate sequence and block ATPase activity, or by inhibiting cofactor binding. The ability to inhibit specific PKC isozymes is limited.

Tamoxifen, a protein kinase C inhibitor with anti-estrogen activity, is currently a standard treatment for hormone-dependent breast cancer. The use of this compound may increase the risk of developing cancer in other tissues such as the endometrium. Raloxifene, a related compound, has been shown to protect against osteoporosis. The tissue specificity of inhibitors must be considered when identifying therapeutic targets.

The cyclin-dependent protein kinases (CDKs) are another group of STKs that control the progression of cells through the cell cycle. Cyclins are small regulatory proteins that act by binding to and activating CDKs that then trigger various phases of the cell cycle by phosphorylating and activating selected proteins involved in the mitotic process. CDKs are unique in that they require multiple inputs to become activated. In addition to the binding of cyclin, CDK activation requires the phosphorylation of a specific threonine residue and the dephosphorylation of a specific tyrosine residue.

Cellular inhibitors of CDKs also play a major role in cell cycle progression. Alterations in the expression, function, and structure of cyclin and CDK are encountered in the cancer phenotype. Therefore CDKs may be important targets for new cancer therapeutic agents.

Often chemotherapy resistant cells tend to escape apoptosis. Under certain circumstances, inappropriate CDK activation may even promote apoptosis by encouraging the progression of the cell cycle under unfavorable conditions, i.e., attempting mitosis while DNA damage is largely unrepaired.

Purines and purine analogs act as CDK inhibitors. Flavopiridol (L86-2,275) is a flavonoid that causes 50% growth inhibition of tumor cells at 60 nM (57). It also inhibits EGFR and protein kinase A. Flavopiridel induces apoptosis and inhibits lymphoid, myeloid, colon, and prostate cancer cells grown in vivo as tumor xenografts in nude mice.

Staurosporine and its derivative, UCN-O1, in addition to inhibiting protein kinase C, inhibit cyclin B/CDK (IC₅₀ 3 to 6 nM). Staurosporine is toxic, but its derivative 7-hydroxystaurosporine (UCN1) has anti-tumor properties and is in clinical trials. UCN-01 affects the phosphorylation of CDKs and alters the cell cycle checkpoint functioning. These compounds illustrate that multiple intracellular targets may be affected as the concentration of an inhibitor is increased within cells.

Protein tyrosine kinases, PTKs, specifically phosphorylate tyrosine residues on their target proteins and may be divided into transmembrane, receptor PTKs and non-transmembrane, non-receptor PTKs. Transmembrane protein-tyrosine kinases are receptors for most growth factors. Binding of growth factor to the receptor activates the transfer of a phosphate group from ATP to selected tyrosine side chains of the receptor and other specific proteins. Growth factors (GF) associated with receptor protein-tyrosine kinases (RTK) include epidermal GF, platelet-derived GF, fibroblast GF, hepatocyte GF, insulin and insulin-like GFs, nerve GF, vascular endothelial GF, and macrophage colony stimulating factor.

Inhibitors of RTKs may inhibit the growth and proliferation of such cancers, since RTKs stimulate tumor cell proliferation. Inhibitors of RTKs are also useful in preventing tumor angiogenesis and can eliminate support from the host tissue by targeting RTKs located on vascular cells (e.g., blood vessel endothelial cells and stromal fibroblasts (FGF receptor)).

Increasing knowledge of the structure and activation mechanism of RTKs and the signaling pathways controlled by tyrosine kinases provided the possibility for the development of target-specific drugs and new anti-cancer therapies. Approaches towards the prevention or interception of deregulated RTK signaling include the development of selective components that target either the extracellular ligand-binding domain or the intracellular tyrosine kinase or substrate binding region.

The most successful strategy to selectively kill tumor cells is the use of monoclonal antibodies (mAbs) that are directed against the extracellular domain of RTKs which are critically involved in cancer and are expressed at the surface of tumor cells. In the past years, recombinant antibody technology has made enormous progress in the design, selection and production of new engineered antibodies, and it is possible to generate humanized antibodies, human-mouse chimeric or biospecific antibodies for targeted cancer therapy. Mechanistically, anti-RTK mAbs might work by blocking the ligand-receptor interaction and therefore inhibiting ligand-induced RTK signaling. In addition, by binding of to certain epitopes on the cancer cells, the anti-RTK mAbs induce immune-mediated responses such as opsonization and complement-mediated lysis and trigger antibody-dependent cellular cytotoxicity by macrophages or natural killer cells. In recent years, it became evident that mAbs control tumor growth by altering the intracellular signaling pattern inside the targeted tumor cell, leading to growth inhibition and/or apoptosis. In contrast, biospecific antibodies can bridge selected surface molecules on a target cell with receptors on an effector cell triggering cytotoxic responses against the target cell. Despite the toxicity that has been seen in clinical trials of bispecific antibodies, advances in antibody engineering, characterization of tumor antigens and immunology might help to produce rationally designed bispecific antibodies for anti-cancer therapy.

Another promising approach to inhibit aberrant RTK signaling are small molecule drugs that selectively interfere with the intrinsic tyrosine kinase activity and thereby block receptor autophosphorylation and activation of downstream signal transducers. The tyrphostins, which belong to the quinazolines, are one important group of such inhibitors that compete with ATP for the ATP binding site at the receptor's tyrosine kinase domain and some members have been shown to specifically inhibit the EGFR. Potent and selective inhibitors of receptors involved in neovascularization have been developed and are now undergoing clinical evaluation. Using the advantages of structure-based drug design, crystallographic structure information, combinatorial chemistry and high-throughput screening, new structural classes of tyrosine kinase inhibitors with increased potency and selectivity, higher in vitro and in vivo efficacy and decreased toxicity have emerged.

Recombinant immunotoxins provide another possibility of target-selective drug design. They are composed of a bacterial or plant toxin either fused or chemically conjugated to a specific ligand such as the variable domains of the heavy and light chains of mAbs or to a growth factor. Immunotoxins either contain the bacterial toxins Pseudomouas exotoxin A or diphtheria toxin or the plant toxins ricin A or clavin. These recombinant molecules can selectively kill their target cells when internalized after binding to specific cell surface receptors.

The use of antisense oligonucleotides represents another strategy to inhibit the activation of RTKs. Antisense oligonucleotides are short pieces of synthetic DNA or RNA that are designed to interact with the mRNA to block the transcription and thus the expression of specific-target proteins. These compounds interact with the mRNA by Watson-Crick base-pairing and are therefore highly specific for the target protein. Several preclinical and clinical studies suggest that antisense therapy might be therapeutically useful for the treatment of solid tumors

A variety of successful target specific drugs such as mAbs and RTK inhibitors have been developed and are currently evaluated in clinical trials. Table 2 summarizes the most successful drugs against receptor tyrosine kinase signaling which are currently evaluated in clinical phases or have already been approved by the FDA.

TABLE 2
RTK Drugs Currently Under Clinical Evaluation

| RTK | Drug | Company | Description | Status |
|---|---|---|---|---|
| EGFR | ZA18539 Iressa | AstraZeneca | TKI that inhibits EGFR | Phase III |
| | | | signaling | |
| EGFR | Cetuximab C225 | ImClone | Mab directed against EGFR | Phase III |
| | | Systems | | |
| EGFR | EGF fusion protein | Seragen | Recombinant diphtheria toxin- | Phase II |
| | | | hEGF fusion protein | |
| HER2 | Trastuzumab | Genetech | Mab directed against HER2 | Approved by |
| | Herceptin | | | the FDA in |
| | | | | 1998 |
| IGF-IR | INX-4437 | INEX USA | Antisense oligonucleotides | Phase I |
| | | | targeting IGR-IR | |
| VEGFR | SU5416 | SUGEN | TKI that inhibits VEGFR2 | Phase II |
| VEGFR/ | SU6668 | SUGEN | RTK inhibition of VEGFR, | Phase I |
| FGFR/ | | | FGFR, and PDGFR | |
| PDGFR | | | | |

Non-receptor PTKs lack transmembrane regions and, instead, form complexes with the intracellular regions of cell surface receptors. Such receptors that function through non-receptor PTKs include those for cytokines, hormones (growth hormone and prolactin) and antigen-specific receptors on T and B lymphocytes.

Many of these PTKs were first identified as the products of mutant oncogenes in cancer cells where their activation was no longer subject to normal cellular controls. In fact, about one third of the known oncogenes encode PTKs, and it is well known that cellular transformation (oncogenesis) is often accompanied by increased tyrosine phosphorylation activity.

Targeting the signaling potential of growth promoting tyrosine kinases such as EGFR, HER2, PDGFR, src, and abl, will block tumor growth while blocking IGF-I and TRK will interfere with tumor cell survival. Inhibiting these kinases will lead to tumor shrinkage and apoptosis. FklI/KDR and src are kinases necessary for neovascularization (angiogenesis) of tumors and inhibition of these will slow tumor growth thereby decreasing metastases.

Inhibitors of RTKs stabilize the tumor in terms of cell proliferation, normal cell loss via apoptosis, and prevent cell migration, invasion and metastases. These drugs are likely to increase the time required for tumor progression, and may inhibit or attenuate the aggressiveness of the disease but may not initially result in measurable tumor regression.

Many tyrosine kinase inhibitors are derived from natural products including flavopiridol, genistem, erbstatin, lavendustin A, staurosporine, and UCN-O1. Inhibitors directed at the ATP binding site are also available. Signals from RTKs can also be inhibited at other target sites such as: nuclear tyrosine kinases, membrane anchors (inhibition of farnesylation) and transcription factors.

An example of cancer arising from a defective tyrosine kinase is a class of ALK positive lymphomas referred to as “ALKomas” which display inappropriate expression of a neural-specific tyrosine kinase, anaplastic lymphoma kinase (ALK).

Iressa (ZD1839) is an orally active selective EGF-R inhibitor. This compound disrupts signaling involved in cancer cell proliferation, cell survival and tumor growth support by the host. The clinical efficacy of this agent shows that it is well tolerated by patients undergoing Phase I/II clinical trials. The compound has shown promising cytotoxicity towards several cancer cell lines.

Since the majority of protein kinases are expressed in the brain, often in neuron-specific fashion, protein phosphorylation must play a key role in the development and function of the vertebrate central nervous system. Thus neuron-specific kinases are well established as targets for the development of pharmacologically active modulators.

In summary, kinase proteins are a major target for drug action and development. Accordingly, it is valuable to the field of pharmaceutical development to identify and characterize kinase proteins that are associated with cancer.

2. CPKGs and CPKG Products As Markers for Cancers

The present invention pertains to the use of the CPKGs listed in Table 1, the transcribed polynucleotides (CPKPN), and the encoded polypeptides (CPKPP) as markers for cancer. Moreover, the use of expression profiles of these genes can indicate the presence of a risk of cancer. These markers are further useful to correlate differences in levels of expression with a poor or favorable prognosis of cancer. The present invention is directed to the use of CPKGs and panels of CPKGs set forth in Table 1 or homologs thereof. For example, panels of the CPKGs can be conveniently arrayed on solid supports, i.e., biochips, such as the GeneChip®, for use in kits. The CPKGs can be used to assess the efficacy of a treatment or therapy of cancer, or as a target for a treatment or therapeutic agent. The CPKGs can also be used to generate vaccines for cancer, to produce antibodies specific to cancer cells, and to construct gene therapy vectors that inhibit tumor growth. Therefore, without limitation as to mechanism, the invention is based in part on the principle that modulation of the expression of the CPKGs of the invention may ameliorate cancer when they are expressed at levels similar or substantially similar to normal (non-diseased) tissue.

In one aspect, the invention provides CPKGs whose level of expression, which signifies their quantity or activity, is correlated with the presence of cancer. In certain embodiments, the invention is performed by detecting the presence of a CPKPN or a CPKPP.

In another aspect of the invention, the expression levels of the CPKGs are determined in a particular subject sample for which either diagnosis or prognosis information is desired. The level of expression of a number of CPKGs simultaneously provides an expression profile, which is essentially a “fingerprint” of the presence or activity of a CPKG or a plurality of CPKGs that is unique to the state of the cell. In certain embodiments, comparison of relative levels of expression is indicative of the severity of cancer, and as such permits for diagnostic and prognostic analysis. Moreover, by comparing relative expression profiles of CPKGs from tissue samples taken at different points in time, e.g., pre- and post-therapy and/or at different time points within a course of therapy, information regarding which genes are important in each of these stages is obtained. The identification of genes that are abnormally expressed in cancer versus normal tissue, as well as differentially expressed genes during cancer development, allows the use of this invention in a number of ways. For example, comparison of expression profiles of CPKGs at different stages of the tumor progression provides a method for long-term prognosis, including survival. In another example mentioned above, the evaluation of a particular treatment regime may be evaluated, including whether a particular drug will act to improve the long-term prognosis in a particular patient.

The discovery of these differential expression patterns for individual or panels of CPKGs allows for screening of test compounds that modulate a particular expression pattern. For example, screening can be done for compounds that will convert an expression profile for a poor prognosis to one for a better prognosis. In certain embodiments, this may be done by making biochips comprising sets of the significant CPKGs, which can then be used in these screens. These methods can also be done on the protein level. Protein expression levels of the CPKGs can be evaluated for diagnostic and prognostic purposes, or used to screen test compounds. For example, in relation to these embodiments, significant CPKGs may comprise CPKGs which are determined to have modulated activity or expression in response to a therapy regime. Alternatively, the modulation of the activity or expression of a CPKG may be correlated with the diagnosis or prognosis of cancer. In addition, the CPKGs can be administered for therapeutic purposes, including the administration of antisense nucleic acids and/or proteins (including CPKPPs, antibodies to CPKPPs and other modulators of CPKPPs).

For example, the CPKG STK-15 has increased expression in cancer tissue samples, relative to control tissue samples. The presence of increased mRNA for this gene (or any other CPKGs set forth in Table 1), or increased levels of the protein products of this gene (or any other CPKGs set forth in Table 1) serve as markers for cancer. Accordingly, amelioration of cancer can be achieved by modulating up-regulated cancer markers, such as STK-15, to normal levels (e.g., levels similar or substantially similar to tissue substantially free of cancer). In many cases, the up-regulated cancer marker is modulated to be similar to a control sample which is taken from a subject or tissue that is substantially free of cancer. Indeed, it is well established that the targets of many cancer therapeutics are kinases.

In another embodiment of the invention, a product of CPKG, either in the form of a polynucleotide or a polypeptide, can be used as a therapeutic compound of the invention. In yet other embodiments, a modulator of CPKG expression or the activity of a CPKG product may be used as a therapeutic compound of the invention. The modulation may also be used in combination with one or more other therapeutic compositions of the invention. Formulation of such compounds into pharmaceutical compositions is described in subsections below. Administration of such a therapeutic may suppress bioactivity of CPKG product, and therefore may be used to ameliorate cancer

3. Sources of CPKG Products

The CPKG products (CPKPNs and CPKPPs) of the invention may be isolated from any body fluid, tissue or cell of a subject. The tissue samples containing one or more of the CPKG products themselves may be useful in the methods of the invention, and one skilled in the art will be cognizant of the methods by which such samples may be conveniently obtained, stored and/or preserved.

Isolated Polynucleotides

One aspect of the invention pertains to isolated polynucleotides. Another aspect of the invention pertains to isolated polynucleotide fragments sufficient for use as hybridization probes to identify a CPKPN in a sample, as well as nucleotide fragments for use as PCR primers of the amplification or mutation of the nucleic acid molecules which encode the CPKPP of the invention.

A CPKPN molecule of the present invention, e.g., a polynucleotide molecule having the nucleotide sequence of one of the CPKGs listed in Table 1, or homologs thereof, or a portion thereof, can be isolated using standard molecular biology techniques and the sequence information provided herein as well as sequence information known in the art. Using all or a portion of the polynucleotide sequence of one of the CPKGs listed Table 1 (or a homolog thereof) as a hybridization probe, a CPKG of the invention or a CPKPN of the invention can be isolated using standard hybridization and cloning techniques.

A CPKPN of the invention can be amplified using cDNA, mRNA or alternatively, genomic DNA, as a template and appropriate oligonucleotide primers according to standard PCR amplification techniques. The polynucleotide so amplified can be cloned into an appropriate vector and characterized by DNA sequence analysis. Furthermore, oligonucleotides corresponding to CPKG nucleotide sequences, or CPKPN of the invention can be prepared by standard synthetic techniques, e.g., using an automated DNA synthesizer.

Alternatively, there are numerous amplification techniques for obtaining a full length coding sequence from a partial cDNA sequence. Within such techniques, amplification is generally performed via PCR. Any of a variety of commercially available kits may be used to perform the amplification step. Primers may be designed using, for example, software well-known in the art. In many embodiments, primers are 22-30 nucleotides in length, have a GC content of at least 50% and anneal to the target sequence at temperatures of about 68° C. to 72° C. The amplified region may be sequenced as described above, and overlapping sequences assembled into a contiguous sequence.

One such amplification technique is inverse PCR, which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are subjected to a second round of amplification with the same linker primer and a second primer specific to the known region. A variation on this procedure, which employs two primers that initiate extension in opposite directions from the known sequence, is described in WO96/38591.

Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic., 1:11-19, 1991) and walking PCR (Parker et al., Nucl. Acids. Res., 19:3055-60, 1991). Other methods using amplification may also be employed to obtain a full length cDNA sequence.

In certain instances, it is possible to obtain a full length cDNA sequence by analysis of sequences provided in an expressed sequence tag (EST) database, such as that available from GenBanK. Searches for overlapping ESTs may generally be performed using well-known programs (e.g., BLAST searches), and such ESTs may be used to generate a contiguous full length sequence. Full length DNA sequences may also be obtained by analysis of genomic fragments.

In another embodiment, an isolated polynucleotide molecule of the invention comprises a polynucleotide molecule which is a complement of the nucleotide sequence of a CPKG listed in Table 1. A polynucleotide molecule which is complementary to such a nucleotide sequence is one which is sufficiently complementary to the nucleotide sequence such that it can hybridize to the nucleotide sequence, thereby forming a stable duplex.

The polynucleotide molecule of the invention, moreover, can comprise sequences corresponding to only a portion of the polynucleotide sequence of a CPKG, for example, a fragment which can be used as targets for developing agents that modulate a CPKPP-mediated activity or as a probe or primer. A biological active portion of a CPKPP may include a fragment of a CPKPP comprising an amino acid that includes fewer amino acids than the full length CPKPP, and exhibits at least one activity of the CPKPP. A biologically active portion of a CPKPP comprises a domain or motif with at least one activity of the CPKPP. The probe/primer typically comprises substantially purified oligonucleotide. The oligonucleotide typically comprises a region of nucleotide sequence that hybridizes under stringent conditions to at least about 7, 10, 15, 25, 50, 75, 100, 125, 150, 175, 200, 225, 250, 275, 300, 325, 350, 400 or more consecutive nucleotides of a CPKG, or a CPKPN.

Probes based on the nucleotide sequence of a CPKG or of a CPKPN can be used to detect transcripts or genomic sequences corresponding to the CPKG and/or CPKPP. In some embodiments, the probe comprises a label group attached thereto, e.g., the label group can be a radioisotope, a fluorescent compound, an enzyme, or an enzyme co-factor. Such probes can be used as a part of a diagnostic test kit for identifying cells or tissue which misexpress (e.g., over- or under-express) a CPKG polynucleotide or polypeptide of the invention, or which have greater or fewer copies of a CPKG. For example, the level of a CPKG product in a sample of cells from a subject may be detected, the amount of polypeptide or mRNA transcript of a CPKG may be determined, or the presence of mutations or deletions of a CPKG of the invention may be assessed.

The invention further encompasses polynucleotide molecules that differ from the polynucleotide sequences of the CPKGs listed in Table 1 due to degeneracy of the genetic code but encode the same proteins encoded by CPKGs shown in Table 1.

The invention also encompasses homologs of the CPKGs listed in Table 1 of other species. Gene homologs are well understood in the art and are available using databases or search engines such as the Pubmed-Entrez database.

The invention also encompasses polynucleotide molecules which are structurally different from the molecules described above (i.e., which have a slight altered sequence), but which have substantially the same properties as the molecules above (e.g., encoded amino acid sequences, or which are changed only in non-essential amino acid residues). Such molecules include allelic variants, and are described in greater detail in subsections herein.

In addition to the nucleotide sequences of the CPKGs listed in Table 1, it will be appreciated by those skilled in the art that DNA sequence polymorphisms leading to changes in the amino acid sequences of the proteins encoded by the CPKGs listed in Table 1 may exist within a population (e.g., the human population). These polymorphic DNA sequences are also encompassed by the present invention. Such genetic polymorphism in the CPKGs listed in Table 1 may exist among individuals within a population due to natural allelic variation. An allele is one of a group of genes which occur alternatively at a given genetic locus. In addition, it will be appreciated that DNA polymorphisms that affect RNA expression levels can also exist and may affect the overall expression level of that gene (e.g., by affecting regulation or degradation). An allelic variant includes a nucleotide sequence which occurs at a given locus and to a polypeptide encoded by the nucleotide sequence.

Polynucleotide molecules corresponding to natural allelic variants and homologs of the CPKGs can be isolated based on their homology to the CPKGs listed in Table 1, using the cDNAs disclosed herein, or a portion thereof, as a hybridization probe according to standard hybridization techniques. Stringency of a hybridization reaction refers to the difficulty with which any two nucleic acid molecules will hybridize to one another. The present invention also includes polynucleotides capable of hybridizing under reduced stringency conditions, stringent conditions, or highly stringent conditions, to polynucleotides described herein. Examples of stringency conditions are shown in Table 3 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 3
Stringency Conditions

| Stringency | Poly-nucleotide | Hybrid | Hybridization | Wash Temp. |
|---|---|---|---|---|
| Condition | Hybrid | Length (bp)¹ | Temperature and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{F}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | <50 | T_{N}*; 6xSSC | T_{N}*; 6xSSC |
| O | DNA:RNA | >50 | 55° C.; 4xSSC -or- | 55° C.; 2xSSC |
| | | | 42° C.; 6xSSC, 50% formamide | |
| P | DNA:RNA | <50 | T_{P}*; 6xSSC | T_{P}*; 6xSSC |
| Q | RNA:RNA | >50 | 60° C.; 4xSSC -or- | 60° C.; 2xSSC |
| | | | 45° C.; 6xSSC, 50% formamide | |
| R | RNA:RNA | <50 | T_{R}*; 4xSSC | T_{R}*; 4xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete. | | | | |
| T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) | | | | |
| # ⁺ 4(# of G + C bases). For hybrids between 18 and 49 base pairs in length, Tₘ(° C.) = 81.5 + 16.6(log₁₀Na⁺) + 0.41(% G⁺C) − (600/N), where N is the number of bases in the hybrid, and Na⁺ is the concentration of sodium ions in the hybridization buffer (Na⁺ for 1xSSC = 0.165M). | | | | |

Polynucleotide molecules corresponding to natural allelic variants and homologs of the CPKGs of the invention can further be isolated by mapping to the same chromosome or locus as the CPKGs of the invention.

In another embodiment, an isolated polynucleotide molecule of the invention is at least 15, 20, 25, 30, 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000 or more nucleotides in length and hybridizes under stringent conditions to a polynucleotide molecule corresponding to a nucleotide sequence of a CPKG of the invention. In one embodiment, an isolated polynucleotide molecule of the invention that hybridizes under stringent conditions to the sequence of one of the CPKGs set forth in Table 1 corresponds to a naturally-occurring polynucleotide molecule.

In addition to naturally-occurring CPKG allelic variants that may exist in the population, the skilled artisan will further appreciate that changes can be introduced by mutation into the nucleotide sequences of the CPKGs of the invention, thereby leading to changes in the amino acid sequence of the encoded proteins, without altering the functional activity of these proteins. For example, nucleotide substitutions leading to amino acid substitutions at “non-essential” amino acid residues can be made. A “non-essential” amino acid residue is a residue that can be altered from the wild-type sequence of a protein without altering the biological activity, whereas an “essential” amino acid residue is required for biological activity. For example, amino acid residues that are conserved among allelic variants or homologs of a gene (e.g., among homologs of a gene from different species) are predicted to be particularly unamenable to alteration.

Accordingly, another aspect of the invention pertains to CPKPP variants that contain changes in amino acid residues that are not essential for activity. Such variants differ in amino acid sequence from the original CPKPP encoded by the CPKG listed in Table 1, yet retain biological activity of the corresponding CPKPP. In one embodiment, the variant comprises an amino acid sequence at least about 60%, 65%, 70%, 75%, 80%, 85%, 90%, 95%, 98% or more homologous to a CPKPP of the invention.

In yet other aspects of the invention, polynucleotides of a CPKG may comprise one or more mutations. An isolated polynucleotide molecule encoding a mutated CPKPP can be created by introducing one or more nucleotide substitutions, additions or deletions into the nucleotide sequence of the gene encoding the CPKPP, such that one or more amino acid substitutions, additions or deletions are introduced into the encoded protein. Such techniques are well-known in the art. Mutations can be introduced into the CPKG polynucleotide of the invention (e.g., a CPKG listed in Table 1) by standard techniques, such as site-directed mutagenesis and PCR-mediated mutagenesis. In many instances, conservative amino acid substitutions are made at one or more predicted non-essential amino acid residues. Alternatively, mutations can be introduced randomly along all or part of a coding sequence of a CPKG of the invention, such as by saturation mutagenesis. The resultant mutants can be screened for biological activity to identify mutants that retain activity. Following mutagenesis, the encoded protein can be expressed recombinantly and the activity of the protein can be determined.

A polynucleotide may be further modified to increase stability in vivo. Possible modifications include, but are not limited to, the addition of flanking sequences at the 5′ and/or 3′ ends; the use of phosphorothioate or 2-o-methyl rather than phosphodiester linkages in the backbone; and the inclusion of nontraditional bases such as inosine, queosine and wybutosine, as well as acetyl-methyl-, thio- and other modified forms of adenine, cytidine, guanine, thymine and uridine.

Another aspect of the invention pertains to isolated polynucleotide molecules that are antisense to the CPKGs of the invention. An “antisense” polynucleotide comprises a nucleotide sequence which is complementary to a “sense” polynucleotide encoding a protein, e.g., complementary to the coding strand of a double-stranded cDNA molecule or complementary to an mRNA sequence. Accordingly, an antisense polynucleotide can form hydrogen bonds to a sense polynucleotide. The antisense polynucleotide can be complementary to an entire coding strand of a CPKG of the invention or to only a portion thereof. In one embodiment, an antisense polynucleotide molecule is antisense to a “coding region” of the coding strand of a nucleotide sequence of the invention. In another embodiment, the antisense polynucleotide molecule is antisense to a “noncoding region” of the coding strand of a nucleotide sequence of the invention.

Antisense polynucleotides of the invention can be designed according to the rules of Watson and Crick base pairing. The antisense polynucleotide molecule can be complementary to the entire coding region of an mRNA corresponding to a gene of the invention. The antisense polynucleotide molecule can also be an oligonucleotide which is antisense to only a portion of the coding or noncoding region. An antisense oligonucleotide can be, for example, about 5, 10, 15, 20, 25, 30, 35, 40, 45 or 50 nucleotides in length. An antisense polynucleotide of the invention can be constructed using chemical synthesis and enzymatic ligation reactions known in the art. For example, an antisense polynucleotide can be chemically synthesized using naturally occurring nucleotides or variously modified nucleotides designed to increase the biological stability of the molecules or to increase the physical stability of the duplex formed between the antisense and sense polynucleotides, e.g., phosphorothioate derivatives and acridine substituted nucleotides can be used. Examples of modified nucleotides which can be used to generate the antisense polynucleotide include 5-fluorouracil, 5-bromouracil, 5-chlorouracil, 5 iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladen4exine, unacil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3)w, and 2,6-diaminopurine. Alternatively, the antisense polynucleotide can be produced biologically using an expression vector into which a polynucleotide has been subcloned in an antisense orientation (i.e., RNA transcribed from the inserted polynucleotide will be of an antisense orientation to a target polynucleotide of interest, described further in the following subsection).

The antisense polynucleotide molecules of the invention are administered to a subject or generated in situ such that they hybridize with or bind to cellular mRNA and/or genomic DNA encoding a CPKPP of the invention to thereby inhibit expression of the protein, e.g., by inhibiting transcription and/or translation. The hybridization may occur based on conventional nucleotide complementarity to form a stable duplex or, in the cases of an antisense polynucleotide molecule which binds to DNA duplexes, through specific interactions in the major groove of the DNA double helix. An example of a route of administration of antisense polynucleotide molecules of the invention is direct injection at a tissue site (e.g., intestine). Alternatively, antisense polynucleotide molecules can be modified to target selected cells and then administered systemically. For systemic administration, antisense molecules can be modified such that they specifically bind to receptors or antigens expressed on a selected cell surface, e.g., by linking the antisense polynucleotide molecules to peptides or antibodies which bind to cell surface receptors or antigens. The antisense polynucleotide molecules can also be delivered to cells using the vectors described herein. To achieve sufficient intracellular concentrations of the antisense molecules, vector constructs in which the antisense polynucleotide molecule is placed under the control of a strong promoter, such as pol II or pol III promoter, may be employed.

In yet another embodiment, the antisense polynucleotide molecule of the invention is an α-anomeric polynucleotide molecule. An α-anomeric polynucleotide molecule forms specific double-stranded hybrids with complementary RNA in which, contrary to the usual β-units, the strands run parallel to each other (Gaultier et al., Polynucleotides. Res., 15:6625-6641, 1987). The antisense polynucleotide molecule can also comprise a 2′-o-methylribonucleotide or a chimeric RNA-DNA analogue.

In still another embodiment, an antisense polynucleotide of the invention is a ribozyme. Ribozymes are catalytic RNA molecules with ribonuclease activity which are capable of cleaving a single-stranded polynucleotide, such as an mRNA, to which they have a complementary region. Thus, ribozymes (e.g., hammerhead ribozymes) can be used to catalytically cleave mRNA transcripts of the CPKGs to thereby inhibit translation of said mRNA. A ribozyme having specificity for a CPKPN can be designed based upon the nucleotide sequence of a gene of the invention, disclosed herein. For example, a derivative of a Tetrahymena L-19 IVS RNA can be constructed in which the nucleotide sequence of the active site is complementary to the nucleotide sequence to be cleaved in a CPKG protein-encoding mRNA. Alternatively, mRNA transcribed from a gene of the invention can be used to select a catalytic RNA having a specific ribonuclease activity from a pool of RNA molecules. Alternatively, expression of a CPKG of the invention can be inhibited by targeting the regulatory region of these genes (e.g., the promoter and/or enhancers) with complementary nucleotide sequences that will form triple helical structures with the target sequence to prevent transcription of the gene in target cells.

Expression of the CPKGs of the invention can also be inhibited using RNA interference (“RNAᵢ”). This is a technique for post-transcriptional gene silencing (“PTGS”), in which target gene activity is specifically abolished with cognate double-stranded RNA (“dsRNA”). RNAᵢ resembles in many aspects PTGS in plants and has been detected in many invertebrates including trypanosome, hydra, planaria, nematode and fruit fly (Drosophila melanogaster). It may be involved in the modulation of transposable element mobilization and antiviral state formation. RNAᵢ technology is disclosed in U.S. Pat. No. 5,919,619 and PCT Publication Nos. WO99/14346, WO01/70949, WO01/36646, WO00/63364, WO00/44895, WO01/75164, WO01/92513, WO01/68836 and WO01/29058. Basically, dsRNA of at least about 21 nucleotides, homologous to the target gene, is introduced into the cell and a sequence specific reduction in gene activity is observed For example, in mammalian cells, introduction of long dsRNA can initiate a potent antiviral response, exemplified by nonspecific inhibition of protein synthesis and RNA degradation. RNA interference provides a mechanism of gene silencing at the mRNA level. In recent years, RNAi has become an endogenous and potent gene-specific silencing technique that uses double-stranded RNAs (dsRNA) to mark a particular transcript for degradation in vivo. It also offers an efficient and broadly applicable approach for gene knock-out. In addition, RNAi technology can be used for therapeutic purposes. For example, RNAi targeting Fas-mediated apoptosis has been shown to protect mice from fulminant hepatitis.

Sequences capable of inhibiting gene expression by RNA interference can have any desired length. For instance, the sequence can have at least 10, 15, 20, 25, or more consecutive nucleotides. The sequence can be dsRNA or any other type of polynucleotide, provided that the sequence can form a functional silencing complex to degrade the target mRNA transcript.

In one embodiment, the sequence comprises or consists of a short interfering RNA (siRNA). The siRNA can be, for example, dsRNA having 19-25 nucleotides. siRNAs can be produced endogenously by degradation of longer dsRNA molecules by an RNase III-related nuclease called Dicer. siRNAs can also be introduced into a cell exogenously or by transcription of an expression construct. Once formed, the siRNAs assemble with protein components into endoribonuclease-containing complexes known as RNA-induced silencing complexes (RISCs). An ATP-generated unwinding of the siRNA activates the RISCs, which in turn target the complementary mRNA transcript by Watson-Crick base-pairing, thereby cleaving and destroying the mRNA. Cleavage of the mRNA takes place near the middle of the region bound by the siRNA strand. This sequence-specific mRNA degradation results in gene silencing.

At least two ways can be employed to achieve siRNA-mediated gene silencing. First, siRNAs can be synthesized in vitro and introduced into cells to transiently suppress gene expression. Synthetic siRNA provides an easy and efficient way to achieve RNAi. siRNA are duplexes of short mixed oligonucleotides which can include, for example, 19 nucleotides with symmetric dinucleotide 3′ overhangs. Using synthetic 21 bp siRNA duplexes (e.g., 19 RNA bases followed by a UU or dTdT 3′ overhang), sequence-specific gene silencing can be achieved in mammalian cells. These siRNAs can specifically suppress targeted gene translation in mammalian cells without activation of DNA-dependent protein kinase (PKR) by longer dsRNA, which may result in non-specific repression of translation of many proteins.

Second, siRNAs can be expressed in vivo from vectors. This approach can be used to stably express siRNAs in cells or transgenic animals. In one embodiment, siRNA expression vectors are engineered to drive siRNA transcription from polymerase III (pol III) transcription units. Pol III transcription units are suitable for hairpin siRNA expression, since they deploy a short AT rich transcription termination site that leads to the addition of 2 bp overhangs (e.g., UU) to hairpin siRNAs—a feature that is helpful for siRNA function. The Pol III expression vectors can also be used to create transgenic mice that express siRNA.

In another embodiment, siRNAs can be expressed in a tissue-specific manner. Under this approach, long double-stranded RNAs (dsRNAs) are first expressed from a promoter (such as CMV (pol II)) in the nuclei of selected cell lines or transgenic mice. The long dsRNAs are processed into siRNAs in the nuclei (e.g., by Dicer). The siRNAs exit from the nuclei and mediate gene-specific silencing. A similar approach can be used in conjunction with tissue-specific promoters to create tissue-specific knockdown mice.

Any 3′ dinucleotide overhang, such as UU, can be used for siRNA design. In some cases, G residues in the overhang are avoided because of the potential for the siRNA to be cleaved by RNase at single-stranded G residues.

With regard to the siRNA sequence itself, it has been found that siRNAs with 30-50% GC content can be more active than those with a higher G/C content in certain cases. Moreover, since a 4-6 nucleotide poly(T) tract may act as a termination signal for RNA pol III, stretches of >4 Ts or As in the target sequence may be avoided in certain cases when designing sequences to be expressed from an RNA pol III promoter. In addition, some regions of mRNA may be either highly structured or bound by regulatory proteins. Thus, it may be helpful to select siRNA target sites at different positions along the length of the gene sequence. Finally, the potential target sites can be compared to the appropriate genome database (human, mouse, rat, etc.). Any target sequences with more than 16-17 contiguous base pairs of homology to other coding sequences may be eliminated from consideration in certain cases.

In one embodiment, siRNA is designed to have two inverted repeats separated by a short spacer sequence and end with a string of Ts that serve as a transcription termination site. This design produces an RNA transcript that is predicted to fold into a short hairpin siRNA. The selection of siRNA target sequence, the length of the inverted repeats that encode the stem of a putative hairpin, the order of the inverted repeats, the length and composition of the spacer sequence that encodes the loop of the hairpin, and the presence or absence of 5′-overhangs, can vary to achieve desirable results.

The siRNA targets can be selected by scanning an mRNA sequence for AA dinucleotides and recording the 19 nucleotides immediately downstream of the AA. Other methods can also been used to select the siRNA targets. In one example, the selection of the siRNA target sequence is purely empirically determined (see e.g., Sui et al, Proc. Natl. Acad. Sci. USA 99: 5515-5520, 2002), as long as the target sequence starts with GG and does not share significant sequence homology with other genes as analyzed by BLAST search. In another example, a more elaborate method is employed to select the siRNA target sequences. This procedure exploits an observation that any accessible site in endogenous mRNA can be targeted for degradation by synthetic oligodeoxyribonucleotide/RNase H method (Lee et al, Nature Biotechnology 20:500-505, 2002).

In another embodiment, the hairpin siRNA expression cassette is constructed to contain the sense strand of the target, followed by a short spacer, the antisense strand of the target, and 5-6 Ts as transcription terminator. The order of the sense and antisense strands within the siRNA expression constructs can be altered without affecting the gene silencing activities of the hairpin siRNA. In certain instances, the reversal of the order may cause partial reduction in gene silencing activities.

The length of nucleotide sequence being used as the stem of siRNA expression cassette can range, for instance, from 19 to 29. The loop size can range from 3 to 23 nucleotides. Other lengths and/or loop sizes can also be used.

In yet another embodiment, a 5′ overhang in the hairpin siRNA construct can be used, provided that the hairpin siRNA is functional in gene silencing. In one example, the 5′ overhang includes about 6 nucleotide residues.

In still yet another embodiment, the target sequences for RNAi are 21-mer or 20-mer sequence fragments selected from CPKG coding sequences, such as SEQ ID NOS:1-44. The target sequences can be selected from either ORF regions or non-ORF regions. The 5′ end of each target sequence has dinucleotide “NA,” where “N” can be any base and “A” represents adenine. The remaining 19-mer or 18-mer sequence has a GC content of between 30% and 65%. In many examples, the remaining 19-mer or 18-mer sequence does not include any four consecutive A or T (i.e., AAAA or TTTT), three consecutive G or C (i.e., GGG or CCC), or seven “GC” in a row. Examples of the target sequences prepared using the above-described criteria (“Relaxed Criteria”) are illustrated in Table 4. Each target sequence in Table 4 has SEQ ID NO:3n−1, and the corresponding siRNA sense and antisense strands have SEQ ID NO:3n and SEQ ID NO:3n+1, respectively, where n is a positive integer. Antisense strand sequences (SEQ ID NO:3n+1) are presented in the 3′ to 5′ direction. For each CPKG coding sequence (SEQ ID NOS:1-44), multiple target sequences can be selected.

Additional criteria can be used for RNAi target sequence design. In one example, the GC content of the remaining 19-mer or 18-mer sequence is limited to between 35% and 55%, and any 19-mer or 18-mer sequence having three consecutive A or T (i.e., AAA or TTT) or a palindrome sequence with 5 or more bases is excluded. In addition, the 19-mer or 18-mer sequence can be selected to have low sequence homology to other human genes. In one embodiment, potential target sequences are searched by BLASTN against NCBI's human UniGene cluster sequence database. The human UniGene database contains non-redundant sets of gene-oriented clusters. Each UniGene cluster includes sequences that represent a unique gene. 19-mer/18-mer sequences producing no hit to other human genes under the BLASTN search can be selected. During the search, the e-value may be set at a stringent value (such as “1”). Furthermore, the target sequence can be selected from the ORF region, and is at least 75-bp from the start and stop codons. Examples of the target sequences prepared using these criteria (“Stringent Criteria”) are demonstrated in Table 4. siRNA sense and antisense sequences (SEQ ID NO:3n and SEQ ID NO:3n+1, respectively) for each target sequence (SEQ ID NO:3n−1) are also provided. Antisense strand sequences (SEQ ID NO:3n+1) are presented in the 3′ to 5′ direction.

TABLE 4
RNAi Target Sequences and siRNA Sequences for CPKGs

| | Relaxed Criteria | Stringent Criteria |
|---|---|---|
| SEQ ID NO | (target seq.: SEQ ID NO: 3n − 1; | (target seq.: SEQ ID NO: 3n − 1; |
| (CPKG | siRNA sense seq.: SEQ ID NO: 3n; | siRNA sense seq.: SEQ ID NO: 3n; |
| coding seq.) | siRNA antisense seq.: SEQ ID NO: 3n + 1) | siRNA antisense seq.: SEQ ID NO: 3n + 1) |
| 1 | SEQ ID NOS: 89-3,829 | SEQ ID NOS: 3,830-4,717 |
| 2 | SEQ ID NOS: 4,718-5,950 | SEQ ID NOS: 5,951-6,067 |
| 3 | SEQ ID NOS: 6,068-7,723 | SEQ ID NOS: 7,724-8,029 |
| 4 | SEQ ID NOS: 8,030-8,443 | SEQ ID NOS: 8,444-8,500 |
| 5 | SEQ ID NOS: 8,501-9,349 | |
| 6 | SEQ ID NOS: 9,350-10,396 | SEQ ID NOS: 10,397-10,468 |
| 7 | SEQ ID NOS: 10,469-10,939 | SEQ ID NOS: 10,940-10,987 |
| 8 | SEQ ID NOS: 10,988-11,422 | SEQ ID NOS: 11,423-11,476 |
| 9 | SEQ ID NOS: 11,477-11,710 | |
| 10 | SEQ ID NOS: 11,711-12,037 | SEQ ID NOS: 12,038-12,043 |
| 11 | SEQ ID NOS: 12,044-12,574 | SEQ ID NOS: 12,575-12,730 |
| 12 | SEQ ID NOS: 12,731-13,450 | SEQ ID NOS: 13,451-13,576 |
| 13 | SEQ ID NOS: 13,577-15,277 | SEQ ID NOS: 15,278-15,442 |
| 14 | SEQ ID NOS: 15,443-15,658 | |
| 15 | SEQ ID NOS: 15,659-16,294 | SEQ ID NOS: 16,295-16,297 |
| 16 | SEQ ID NOS: 16,298-16,729 | SEQ ID NOS: 16,730-16,741 |
| 17 | SEQ ID NOS: 16,742-18,592 | SEQ ID NOS: 18,593-18,742 |
| 18 | SEQ ID NOS: 18,743-19,651 | SEQ ID NOS: 19,652-19,735 |
| 19 | SEQ ID NOS: 19,736-21,373 | SEQ ID NOS: 21,374-21,622 |
| 20 | SEQ ID NOS: 21,623-22,375 | SEQ ID NOS: 22,376-22,489 |
| 21 | SEQ ID NOS: 22,490-23,338 | SEQ ID NOS: 23,339-23,533 |
| 22 | SEQ ID NOS: 23,534-24,172 | SEQ ID NOS: 24,173-24,214 |
| 23 | SEQ ID NOS: 24,215-25,198 | SEQ ID NOS: 25,199-25,360 |
| 24 | SEQ ID NOS: 25,361-26,278 | SEQ ID NOS: 26,279-26,446 |
| 25 | SEQ ID NOS: 26,447-26,986 | SEQ ID NOS: 26,987-27,028 |
| 26 | SEQ ID NOS: 27,029-27,988 | SEQ ID NOS: 27,989-28,162 |
| 27 | SEQ ID NOS: 28,163-28,468 | SEQ ID NOS: 28,469-28,486 |
| 28 | SEQ ID NOS: 28,487-29,218 | SEQ ID NOS: 29,219-29,323 |
| 29 | SEQ ID NOS: 29,324-29,941 | SEQ ID NOS: 29,942-30,043 |
| 30 | SEQ ID NOS: 30,044-30,217 | |
| 31 | SEQ ID NOS: 30,218-30,763 | SEQ ID NOS: 30,764-30,877 |
| 32 | SEQ ID NOS: 30,878-31,504 | SEQ ID NOS: 31,505-31,540 |
| 33 | SEQ ID NOS: 31,541-36,385 | SEQ ID NOS: 36,386-37,465 |
| 34 | SEQ ID NOS: 37,466-38,731 | SEQ ID NOS: 38,732-38,947 |
| 35 | SEQ ID NOS: 38,948-39,466 | SEQ ID NOS: 39,467-39,496 |
| 36 | SEQ ID NOS: 39,497-40,159 | SEQ ID NOS: 40,160-40,288 |
| 37 | SEQ ID NOS: 40,289-40,570 | SEQ ID NOS: 40,571-40,573 |
| 38 | SEQ ID NOS: 40,574-42,196 | SEQ ID NOS: 42,197-42,313 |
| 39 | SEQ ID NOS: 42,314-43,150 | SEQ ID NOS: 43,151-43,222 |
| 40 | SEQ ID NOS: 43,223-43,528 | SEQ ID NOS: 43,529-43,564 |
| 41 | SEQ ID NOS: 43,565-44,392 | SEQ ID NOS: 44,393-44,404 |
| 42 | SEQ ID NOS: 44,405-45,721 | SEQ ID NOS: 45,722-45,946 |
| 43 | SEQ ID NOS: 45,947-46,879 | SEQ ID NOS: 46,880-46,999 |
| 44 | SEQ ID NOS: 47,000-48,358 | SEQ ID NOS: 48,359-48,640 |

The effectiveness of the siRNA sequences can be evaluated using various methods known in the art. For instance, a siRNA sequence of the present invention can be introduced into a cell that expresses a CPKG. The polypeptide or mRNA level of the CPKG in the cell can be detected. A substantial change in the expression level of the CPKG before and after the introduction of the siRNA sequence is indicative of the effectiveness of the siRNA sequence in suppressing the expression of the CPKG. In one example, the expression levels of other genes are also monitored before and after the introduction of the siRNA sequence. A siRNA sequence which has inhibitory effect on the CPKG expression but does not significantly affect the expression of other genes can be selected. In another example, multiple siRNA or other RNAi sequences can be introduced into the same target cell. These siRNA or RNAi sequences specifically inhibit the CPKG gene expression but not the expression of other genes. In yet another example, siRNA or other RNAi sequences that inhibit the expression of both the CPKG gene and other gene or genes can be used.

In yet another embodiment, the polynucleotide molecules of the present invention can be modified at the base moiety, sugar moiety or phosphate backbone to improve, e.g., the stability, hybridization, or solubility of the molecule. For example, the deoxyribose phosphate backbone of the polynucleotide molecules can be modified to generate peptide polynucleotides (PNAs). In PNAs, the deoxyribose phosphate backbone is replaced by a pseudopeptide backbone. The neutral backbone of PNAs has been shown to allow for specific hybridization to DNA and RNA under conditions of low ionic strength. The synthesis of PNA oligomers can be performed using standard solid phase peptide synthesis protocols.

PNAs can be used in therapeutic and diagnostic applications. For example, PNAs can be used as antisense or antigene agents for sequence specific modulation of CPKG expression by inducing transcription or translation arrest or inhibiting replication. PNAs of the polynucleotide molecules of the invention (e.g., set forth in Table 1 or homologs thereof) can also be used in the analysis of single base pair mutations in a gene, (e.g., by PNA-directed PCR clamping), as artificial restriction enzymes when used in combination with other enzymes (e.g., S1 nucleases) or as probes or primers for DNA sequencing or hybridization.

In another embodiment, PNAs can be modified to enhance their stability or cellular uptake by attaching lipophilic or other helper groups to PNA, by the formation of PNA-DNA chimeras, or by the use of liposomes or other techniques of drug delivery known in the art. For example, PNA-DNA chimeras of the polynucleotide molecules of the invention can be generated. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation. The synthesis of PNA-DNA chimeras can be performed. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a spacer between the PNA and the 5′ end of DNA. PNA monomers are then coupled in a stepwise manner to produce a chimeric molecule with a 5′ PNA segment and a 3′ DNA segment. Alternatively, chimeric molecules can be synthesized with a 5′ DNA segment and a 3′ PNA segment.

In other embodiments, the polynucleotide may include other appended groups such as peptides (e.g., for targeting host cell receptors in vivo), or agents facilitating transport across the cell membrane or the blood-kidney barrier (see, e.g., PCT Publication No. WO89/10134). In addition, polynucleotides can be modified with hybridization-triggered cleavage agents or intercalating agents. To this end, the polynucleotide may be conjugated to another molecule (e.g., a peptide, hybridization triggered cross-linking agent, transport agent, or hybridization-triggered cleavage agent). Finally, the polynucleotide may be detectably labeled, either such that the label is detected by the addition of another reagent (e.g., a substrate for an enzymatic label), or is detectable immediately upon hybridization of the nucleotide (e.g., a radioactive label or a fluorescent label).

Isolated Polypeptides

Several aspects of the invention pertain to isolated CPKPPs and biologically active portions thereof, as well as polypeptide fragments suitable for use as immunogens to raise anti-CPKPP antibodies. In one embodiment, native CPKPPs can be isolated from cells or tissue sources by an appropriate purification scheme using standard protein purification techniques.

Standard purification methods include electrophoretic, molecular, immunological and chromatographic techniques, including ion exchange, hydrophobic, affinity, and reverse-phase HPLC chromatography, and chromatofocusing. For example, the CPKPPs may be purified using a standard anti-CPKPP antibody column. Ultrafiltration and diafiltration techniques, in conjunction with protein concentration, are also useful. The degree of purification necessary will vary depending on the use of the CPKPP. In some instances no purification will be necessary.

In another embodiment, CPKPPs or mutated CPKPPs capable of inhibiting normal CPKPP activity (dominant-negative mutants) are produced by recombinant DNA techniques. Alternative to recombinant expression, a CPKPP or mutated CPKPP can be synthesized chemically using standard peptide synthesis techniques.

The invention provides CPKPPs encoded by CPKGs set forth in Table 1 or homologs thereof. In other embodiments, the CPKPP is substantially homologous to a CPKPP encoded by a CPKG listed in Table 1, and retains the functional activity of the CPKPP, yet differs in amino acid sequence due to natural allelic variation or mutagenesis, as described in detail above. Accordingly, in another embodiment, the CPKPP is a protein which comprises an amino acid sequence at least about 60%, 65%, 70%, 75%, 80%, 85%, 90%, 95%, 98% or more homologous to the amino acid sequence encoded by a CPKG listed in Table 1.

When the proteins are “homologs” and “homologous,” the first protein region and the second protein region are compared in terms of identity. To determine the percent identity of two amino acid sequences or of two polynucleotide sequences, the sequences are aligned for optimal comparison purposes (e.g., gaps can be introduced in one or both of a first and a second amino acid or polynucleotide sequence for optimal alignment and non-homologous sequences can be disregarded for comparison purposes). In one embodiment, the length of a reference sequence aligned for comparison purposes is at least 30%, 40%, 50%, 60%, 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or polynucleotide “identity” is equivalent to amino acid or polynucleotide “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman and Wunsch (J. Mol. Biol., 48:444-453, 1970) algorithm which has been incorporated into the GAP program in the GCG software package, using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package, using a NWSgapdna.CMP matrix and a gap weight of 40, 50, 60, 70, or 80 and a length weight of 1,2,3,4,5, or 6.

The polynucleotide and protein sequences of the present invention can further be used as a “query sequence” to perform a search against public databases to, for example, identify other family members or related sequences. Such searches can be performed using BLAST programs available at the BLAST website maintained by the National Center of Biotechnology Information (NCBI), National Library of Medicine, Washington, D.C., USA.

The invention also provides chimeric or fusion CPKPPs. A fusion CPKPP may contain all or a portion of a CPKPP and a fusion partner (non-CPKPP-related polypeptide). In one embodiment, a fusion CPKPP comprises at least one biologically active portion of a CPKPP. The non-CPKPP-related polypeptide can be fused to the N-terminus or C-terminus of the CPKPP-related polypeptide.

A peptide linker sequence may be employed to separate the CPKPP-related polypeptide from non-CPKPP-related polypeptide by a distance sufficient to ensure that each polypeptide folds into its secondary and tertiary structures. Such a peptide linker sequence is incorporated into the fusion protein using standard techniques well-known in the art. Suitable peptide linker sequences may be chosen based on the following factors: (1) their ability to adopt a flexible extended conformation; (2) their inability to adopt a secondary structure that could interact with functional epitopes on the CPKPP-related polypeptide and non-CPKPP-related polypeptide; and (3) the lack of hydrophobic or charged residues that might react with the polypeptide functional epitopes. Exemplary peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene, 40:39-46, 1985; Murphy et al., Proc. Natl. Acad. Sci., USA, 83:8258-8262, 1986; U.S. Pat. No. 4,935,233 and U.S. Pat. No. 4,751,180. The linker sequence may generally be from 1 to about 50 amino acids in length. Linker sequences are not required when the CPKPP-related polypeptide and non-CPKPP-related polypeptide have non-essential N-terminal amino acid regions that can be used to separate the functional domains and prevent steric interference.

In one embodiment, the fusion protein is a glutathione s-transferase (GST)-CPKPP fusion protein in which the CPKPP sequence is fused to the C-terminus of the GST sequences. Such fusion proteins can facilitate the purification of recombinant CPKPPs.

In another embodiment, the fusion protein is a CPKPP containing a heterologous signal sequence at its N-terminus. In certain host cells (e.g., mammalian host cells), expression and/or secretion of CPKPPs can be increased through use of a heterologous signal sequence. Such signal sequences are well-known in the art.

The CPKPP fusion proteins of the invention can be incorporated into pharmaceutical compositions and administered to a subject in vivo. The CPKPP fusion proteins can be used to affect the bioavailability of a CPKPP substrate. Use of CPKPP fusion proteins may be useful therapeutically for the treatment of or prevention of damage caused by, for example, (i) aberrant modification or mutation of a CPKG; (ii) mis-regulation of a CPKG; and (iii) aberrant post-translational modification of a CPKPP.

Moreover, the CPKPP fusion proteins of the invention can be used as immunogens to produce anti-CPKPP antibodies in a subject, to purify CPKPP ligands and in screening assays to identify molecules which inhibit the interaction of a CPKPP with a CPKPP substrate.

CPKPP fusion proteins used as immunogens may comprise a non-CPKPP immunogenic polypeptide. In one embodiment, the immunogenic protein is capable of eliciting a recall response.

In another embodiment, a CPKPP chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation.

In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence. Moreover, many expression vectors are commercially available that already encode a fusion moiety (e.g., a GST polypeptide). A CPKPP-encoding polynucleotide can be cloned into such an expression vector such that the fusion moiety is linked in-frame to the CPKPP.

A signal sequence can be used to facilitate secretion and isolation of the secreted protein or other proteins of interest. Signal sequences are characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a polynucleotide sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods.

Alternatively, the signal sequence can be linked to the protein of interest using a sequence which facilitates purification, such as with a GST domain.

The present invention also pertains to variants of the CPKPPs of the invention which function as either agonists or as antagonists to the CPKPPs. These CPKPP variants differ from their native CPKPPs in one or more substitutions, deletions, additions and/or insertions, such that the activity or immunogenicity of the native polypeptide is not substantially diminished. In one embodiment, a bioactivity of a CPKPP variant or the ability of a variant CPKPP to react with antigen-specific antisera is enhanced or diminished by less than 50% relative to the native polypeptide. In another embodiment, CPKPP variants include variants in which a small portion (e.g., 1-30 amino acids or 5-15 amino acids) has been removed from the N- and/or C-terminal of the mature protein.

In one embodiment, antagonists or agonists of CPKPPs are used as therapeutic agents. For example, antagonists of an up-regulated CPKG that can decrease the activity or expression of such a gene may ameliorate cancer in a subject wherein the CPKG is abnormally increased in level or activity. In this embodiment, treatment of such a subject may comprise administering the antagonists to decrease activity or expression of the targeted CPKG. Variants of the CPKPPs may contain conservative substitutions wherein an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. In addition, a variant CPKPP may also, or alternatively, contain nonconservative changes. For example, a variant CPKPP may differ from its native sequence by substitution, deletion or addition of five amino acids or fewer. A variant CPKPP may also (or alternatively) be modified by, for example, the deletion or addition of amino acids that have minimal influence on the immunogenicity, secondary structure and hydropathic nature of the polypeptide. In one embodiment, a CPKPP variant exhibits at least about 70%, 80%, 90%, 95% or more sequence homology to the original polypeptide. Lastly, a variant CPKPP may include a CPK polypeptide that is modified from the original CPK polypeptide by either natural processes, such as post-translational processing, or by chemical modification techniques that are well-known in the art. Modifications can occur anywhere in the CPK polypeptide, including the peptide backbone, the amino acid side-chains and the amino or carboxyl termini. It will be appreciated that the same type of modification may be present in the same or varying degrees at several sites in a given polypeptide.

In certain embodiments, an agonist of the CPKPPs can retain substantially the same, or a subset, of the biological activities of the naturally occurring form of.a CPKPP or may enhance an activity of a CPKPP. In certain embodiments, an antagonist of a CPKPP can inhibit one or more of the activities of the naturally occurring form of the CPKPP by, for example, competitively modulating an activity of a CPKPP. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the CPKPP.

Mutants of a CPKPP which function as either CPKPP agonists or as CPKPP antagonists can be identified by screening combinatorial libraries of mutants, e.g., truncation mutants, of a CPKPP for CPKPP agonist or antagonist activity. A variegated library of CPKPP variants can be produced by, for example, enzymatically ligating a mixture of synthetic oligonucleotides into gene sequences such that a degenerate set of potential CPKPP sequences is expressible as individual polypeptides, or alternatively, as a set of larger fusion proteins (e.g., for phage display) containing the set of CPKPP sequences therein. There are a variety of methods which can be used to produce libraries of potential CPKPP variants from a degenerate oligonucleotide sequence. Chemical synthesis of a degenerate gene sequence can be performed in an automatic DNA synthesizer, and the synthetic gene is then ligated into an appropriate expression vector. Use of a degenerate set of genes allows for the provision, in one mixture, of all of the sequences encoding the desired set of potential CPKPP sequences. Methods for synthesizing degenerate oligonucleotides are known in the art.

In addition, libraries of fragments of a protein coding sequence corresponding to a CPKPP of the invention can be used to generate a variegated population of CPKPP fragments for screening and subsequent selection of variants of a CPKPP. In one embodiment, a library of coding sequence fragments can be generated by treating a double-stranded PCR fragment of a CPKPP coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By this method, an expression library can be derived which encodes N-terminal, C-terminal and internal fragments of various sizes of the CPKPP.

Several techniques are known in the art for screening gene products of combinatorial libraries made by point mutations or truncation, and for screening cDNA libraries for gene products having a selected property. The most widely used techniques, which are amenable to high-throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify CPKPP variants (Delgrave et al. Protein Engineering, 6:327-331, 1993).

Portions of a CPKPP or variants of a CPKPP having less than about 100 amino acids, and generally less than about 50 amino acids, may also be generated by synthetic means, using techniques well-known to those of ordinary skill in the art. For example, such polypeptides may be synthesized using any of the commercially available solid-phase techniques, such as the Merrifield solid-phase synthesis method, where amino acids are sequentially added to a growing amino acid chain. Equipment for automated synthesis of polypeptides is commercially available from suppliers such as Perkin Elmer/Applied BioSystems Division (Foster City, Calif.), and may be operated according to the manufacturer's instructions.

Methods and compositions for screening for protein inhibitors or activators are known in the art (see U.S. Pat. Nos. 4,980,281, 5,266,464, 5,688,635, and 5,877,007, which are incorporated herein by reference).

Antibodies

In another aspect, the invention includes antibodies that are specific to CPKPPs of the invention or their variants. In one embodiment, the antibodies are, without limitation, monoclonal or humanized.

In another aspect, the invention provides methods of making an isolated hybridoma which produces an antibody useful for diagnosing a patient or animal with cancer. In this method, a CPKPP or its variant is isolated (e.g., by purification from a cell in which it is expressed or by transcription and translation of a polynucleotide encoding the protein in vivo or in vitro using known methods). A vertebrate, such as a mammal (e.g., a mouse, rabbit or sheep), is immunized using the isolated polypeptide or polypeptide fragment. The vertebrate may be immunized at least one additional time with the isolated polypeptide or polypeptide fragment, so that the vertebrate exhibits a robust immune response to the polypeptide or polypeptide fragment. Splenocytes are isolated from the immunized vertebrate and fused with an immortalized cell line to form hybridomas, using any of a variety of methods well-known in the art. Hybridomas formed in this manner are then screened using standard methods to identify one or more hybridomas which produce an antibody which specifically binds with the polypeptide or polypeptide fragment. The invention also includes hybridomas made by this method and antibodies made using such hybridomas.

An isolated CPKPP, or a portion or fragment thereof, can be used as an immunogen to generate antibodies that bind the CPKPP using standard techniques for polyclonal and monoclonal antibody preparation. A full-length CPKPP can be used or, alternatively, the invention provides antigenic peptide fragments of the CPKPP for use as immunogens. The antigenic peptide of a CPKPP comprises at least 8 amino acid residues of an amino acid sequence encoded by a CPKG set forth in Table 1, and encompasses an epitope of a CPKPP such that an antibody raised against the peptide forms a specific immune complex with the CPKPP. In many instances, the antigenic peptide comprises at least 8, 12, 16, 20, or more amino acid residues.

Immunogenic portions (epitopes) may generally be identified using well-known techniques. Such techniques include screening polypeptides for the ability to react with antigen-specific antibodies, antisera and/or T cell lines or clones. When an antisera and antibodies are antigen-specific, they bind to an antigen with a siginifcant binding affinity. In many cases, the binding affinity is equal to or greater than 10⁵ M⁻¹. Such antisera and antibodies may be prepared by using well-known techniques. An epitope of a CPKPP is a portion that reacts with such antisera and/or T cells at a level that is not substantially less than the reactivity of the full length polypeptide (e.g., in an ELISA and/or T cell reactivity assay). Such epitopes may react within such assays at a level that is similar to or greater than the reactivity of the full length polypeptide. Such screens may generally be performed using methods well-known to those of ordinary skill in the art. For example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

Exemplary epitopes encompassed by the antigenic peptide are regions of the CPKPP that are located on the surface of the protein, e.g., hydrophilic regions, as well as regions with high antigenicity.

A CPKPP immunogen typically is used to prepare antibodies by immunizing a suitable subject (e.g., rabbit, goat, mouse or other mammal) with the immunogen. An appropriate immunogenic preparation can contain, for example, recombinantly expressed CPKPP or a chemically synthesized CPKPP. The preparation can further include an adjuvant, such as Freund's complete or incomplete adjuvant, or similar immunostimulatory agent. Immunization of a suitable subject with an immunogenic CPKPP preparation induces a polyclonal anti-CPKPP antibody response. Techniques for preparing, isolating and using antibodies are well-known in the art.

Accordingly, another aspect of the invention pertains to monoclonal or polyclonal anti-CPKPP antibodies. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to CPKPP.

Polyclonal anti-CPKPP antibodies can be prepared as described above by immunizing a suitable subject with a CPKPP. The anti-CPKPP antibody titer in the immunized subject can be monitored over time by standard techniques, such as with an enzyme linked immunosorbent assay (ELISA) using immobilized CPKPP. If desired, the antibody molecules directed against CPKPPs can be isolated from the mammal (e.g., from the blood) and further purified by well-known techniques, such as protein A chromatography, to obtain the IgG fraction. At an appropriate time after immunization, e.g., when the anti-CPKPP antibody titers are highest, antibody-producing cells can be obtained from the subject and used to prepare monoclonal antibodies by standard techniques, such as the hybridoma technique, human B cell hybridoma technique, the EBV-hybridoma technique, or trioma techniques. The technology for producing monoclonal antibody hybridomas is well-known. Briefly, an immortal cell line (typically a myeloma) is fused to lymphocytes (typically splenocytes) from a mammal immunized with a CPKPP immunogen as described above, and the culture supernatants of the resulting hybridoma cells are screened to identify a hybridoma producing a monoclonal antibody that binds to a CPKPP of the invention.

Any of the many well-known protocols used for fusing lymphocytes and immortalized cell lines can be applied for the purpose of generating an anti-CPKPP monoclonal antibody. Moreover, the ordinarily skilled worker will appreciate that there are many variations of such methods which also would be useful.

Alternative to preparing monoclonal antibody-secreting hybridomas, a monoclonal anti-CPKPP antibody can be identified and isolated by screening a recombinant combinatorial immunoglobulin library (e.g., an antibody phase display library) with CPKPP to thereby isolate immunoglobulin library members that bind to a CPKPP. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612).

The anti-CPKPP antibodies also include “Single-chain Fv” or “scFv” antibody fragments. The scFv fragments comprise the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding.

Additionally, recombinant anti-CPKPP antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

Humanized antibodies are particularly desirable for therapeutic treatment of human subjects. Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies), which contain minimal sequence derived from non-human immunoglobulin. Humanized antibodies include human immunoglobulins (recipient antibody) in which residues forming a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the constant regions being those of a human immunoglobulin consensus sequence. In one embodiment, the humanized antibody comprises at least a portion of an immunoglobulin constant region (Fc), typically that of a human immunoglobulin.

Such humanized antibodies can be produced using transgenic mice which are incapable of expressing endogenous immunoglobulin heavy and light chain genes, but which can express human heavy and light chain genes. The transgenic mice are immunized in the normal fashion with a selected antigen, e.g., all or a portion of a polypeptide corresponding to a CPKPP of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies.

Humanized antibodies which recognize a selected epitope can be generated using a technique referred to as “guided selection.” In this approach a selected non-human monoclonal antibody, e.g., a murine antibody, is used to guide the selection of a humanized antibody recognizing the same epitope.

In one embodiment, the antibodies to CPKPP are capable of reducing or eliminating the biological function of CPKPP, as is described below. That is, the addition of anti-CPKPP antibodies (either polyclonal or monoclonal) to CPKPP (or cells containing CPKPP) may reduce or eliminate the CPKPP activity. In one embodiment, at least a 25% decrease in activity is achieved. In another embodiment, at least a 50%, 60%, 70%, 80%, 90%, or 100% decrease in activity is achieved.

An anti-CPKPP antibody can be used to isolate a CPKPP of the invention by standard techniques, such as affinity chromatography or immunoprecipitation. An anti-CPKPP antibody can facilitate the purification of natural CPKPPs from cells and of recombinantly produced CPKPPs expressed in host cells. Moreover, an anti-CPKPP antibody can be used to detect a CPKPP (e.g., in a cellular lysate or cell supernatant on the cell surface) in order to evaluate the abundance and pattern of expression of the CPKPP. Anti-CPKPP antibodies can be used diagnostically to monitor protein levels in tissue as part of a clinical testing procedure, for example, to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Anti-CPKPP antibodies of the invention are also useful for targeting a therapeutic to a cell or tissue comprising the antigen of the anti-CPKPP antibody. For example, a therapeutic such as a small molecule can be linked to the anti-CPKPP antibody in order to target the therapeutic to the cell or tissue comprising the CPKPP antigen. The method is particularly useful in connection with CPKPPs which are surface markers.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional, may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used.

In another embodiment, antibodies to a CPKPP may be used to eliminate the CPKPP-containing cell population in vivo by activating the complement system, by mediating antibody-dependent cellular cytotoxicity (ADCC), or by causing uptake of the antibody coated cells by the receptor-mediated endocytosis (RE) system.

CPKPP-specific Cytotoxic Lymphocytes (T cells)

Another aspect of the invention pertains to immunotherapeutic compositions comprising T cells specific for a CPKPP. Such cells may generally be prepared in vitro or ex vivo, using standard procedures. T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.). Alternatively, T cells may be derived from related or unrelated humans, non-human mammals, cell lines or cultures.

T cells may be stimulated with a CPKPP or polynucleotide encoding a CPKPP and/or an antigen presenting cell (APC) that expresses a CPKPP. Such stimulation is performed under conditions and for a time sufficient to permit the generation of T cells that are specific for the polypeptide. In one embodiment, a CPKPP or polynucleotide encoding a CPKPP is present within a delivery vehicle, such as a microsphere, to facilitate the generation of specific T cells.

T cells are considered to be specific for a CPKPP if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two-fold increase in lysis and/or proliferation, compared to negative controls, indicates T cell specificity. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a tumor polypeptide (e.g., 100 ng/ml-100 μg/ml, or 200 ng/ml-25 μg/ml) for 3-7 days should result in at least a two-fold increase in proliferation of the T cells. Contact as described above for 2-3 hours should result in activation of the T cells, as measured using standard cytokine assays in which a two-fold increase in the level of cytokine release (e.g., TNF or IFNγ) is indicative of T cell activation. T cells that have been activated in response to a CPKPP, polynucleotide encoding a CPKPP, or CPKPPe-expressing APC may be CD4⁺ and/or CD8⁺. Tumor protein-specific T cells may be expanded using standard techniques. Within many embodiments, the T cells are derived from a patient, a related donor or an unrelated donor, and are administered to the patient following stimulation and expansion.

For therapeutic purposes, CD4⁺ or CD8⁺ T cells that proliferate in response to a CPKPP, polynucleotide encoding a CPKPP, or APC can be expanded in number either in vitro or in vivo. Proliferation of such T cells in vitro may be accomplished in a variety of ways. For example, the T cells can be re-exposed to a CPKPP, or a short peptide corresponding to an immunogenic portion of such a polypeptide, with or without the addition of T cell growth factors, such as interleukin-2, and/or stimulator cells that synthesize a CPKPP. Alternatively, one or more T cells that proliferate in the presence of a CPKPP can be expanded in number by cloning. Methods for cloning cells are well-known in the art, and include limiting dilution.

Vaccines

Within certain aspects, CPKPP, CPKPN, CPKPP-specific T cell, CPKPP-presenting APC, and CPKG-containing vectors including, but not limited to, expression vectors and gene delivery vectors, may be utilized as vaccines for cancer. Vaccines may comprise one or more such compounds/cells and an immunostimulant. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes (into which the compound is incorporated). Vaccines within the scope of the present invention may also contain other compounds, which may be biologically active or inactive. For example, one or more immunogenic portions of other tumor antigens may be present, either incorporated into a fusion polypeptide or as a separate compound, within the composition of vaccine.

A vaccine may contain DNA encoding one or more CPKPP or portion of CPKPP, such that the polypeptide is generated in situ. As noted above, the DNA may be present within any of a variety of delivery systems known to those of ordinary skill in the art, including nucleic acid expression vectors, gene delivery vectors, and bacteria expression systems. Numerous gene delivery techniques are well-known in the art. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In one embodiment, the DNA may be introduced using a viral expression system (e.g., vaccinia or other pox virus, retrovirus, or adenovirus), which may involve the use of a non-pathogenic (defective), replication competent virus. Techniques for incorporating DNA into such expression systems are well-known to those of ordinary skill in the art. The DNA may also be “naked,” as described, for example, in Ulmer et al., (Science, 259:1745-1749, 1993). The uptake of naked DNA may be increased by coating the DNA onto biodegradable beads, which are efficiently transported into the cells. A vaccine may comprise both a polynucleotide and a polypeptide component. Such vaccines may provide for an enhanced immune response.

A vaccine may contain pharmaceutically acceptable salts of the polynucleotides and polypeptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

Any of a variety of immunostimulants may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable micro spheres; monophosphoryl lipid A and quil A. Cytokines, such as GM-CSF or IL-2, IL-7, or IL-12, may also be used as adjuvants.

Within the vaccines provided herein, the adjuvant composition can be designed to induce an immune response predominantly of the Th1 type. High levels of Th1-type cytokines (e.g., IFNγ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within one embodiment, in which a response is predominantly Th1-type, the level of Th1-type cytokines will increase to a greater extent than the level of Th2-type cytokines. The levels of these cytokines may be readily assessed using standard assays.

Exemplary adjuvants for use in eliciting a predominantly Th1-type response include, for example, a combination of monophosphoryl lipid A (e.g., 3-de-O-acylated monophosphoryl lipid A (3D-MPL)) together with an aluminum salt. MPL adjuvants are available from Corixa Corporation (Seattle, Wash.). CpG-containing oligonucleotides (in which the CpG dinucleotide is unmethylated) also induce a predominantly Th1 response. Such oligonucleotides are well-known. Immunostimulatory DNA sequences are also described, for example, by Sato et al., Science, 273:352, 1996. Another exemplary adjuvant is a saponin, such as QS21 (Aquila Biopharmaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL as described in WO94/00153, or a less reactogenic composition where the QS21 is quenched with cholesterol, as described in WO96/33739. Other exemplary formulations comprise an oil-in-water emulsion and tocopherol. A particularly potent adjuvant formulation involving QS21, 3D-MPL and tocopherol in an oil-in-water emulsion is described in WO 95/17210.

Other examples of adjuvants include Montamide ISA 720 (Seppic, France), SAF (Chiron, Calif.), ISCOMS (CSL), MF-59 (Chiron, Calif.), the SBAS series of adjuvants (e.g., SBAS-2 or SBAS-4, available from SmithKline Beecham, Rixensart, Belgium), Detox (Ribi ImmunoChem Research Inc., Hamilton, Mont.), RC-529 (Ribi ImmunoChem Research Inc., Hamilton, Mont.) and Aminoalkyl glucosaminide 4-phosphates (AGPs).

Any vaccine provided herein may be prepared using well-known methods that result in a combination of antigen, immune response enhancer and a suitable carrier or excipient. The compositions described herein may be administered as part of a sustained release formulation (i.e., a formulation such as a capsule, sponge or gel (composed of polysaccharides, for example) that effects a slow release of compound following administration). Such formulations may generally be prepared using well-known technology and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable. In one embodiment, the formulation provides a relatively constant level of active component release. Such carriers include microparticles of poly (lactide-co-glycolide), as well as polyacrylate, latex, starch, cellulose and dextran. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254). The amount of active compound contained within a sustained release formulation depends upon the site of implantation, the rate and expected duration of release and the nature of the condition to be treated or prevented.

Any of a variety of delivery vehicles may be employed within vaccines to facilitate production of an antigen-specific immune response that targets cancer cells. Delivery vehicles include antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain embodiments of the present invention use dendritic cells or progenitors thereof as APCs. Dendritic cells are highly potent APCs and have been shown to be effective as a physiological adjuvant for eliciting prophylactic or therapeutic antitumor immunity. In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med., 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well-characterized phenotypes. However, this nomenclature should not be construed to exclude all possible intermediate stages of differentiation. Immature dendritic cells are characterized as APC with a high capacity for antigen uptake and processing, which correlates with the high expression of Fcy receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide encoding a CPKPP (or portion or other variant thereof) such that the CPKPP, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO97/24447, or the gene gun approach described by Mahvi et al., Immunology and Cell Biology, 75:456-460, 1997. Antigen loading of dendritic cells may be achieved by incubating dendritic cells or progenitor cells with the CPKPPs, DNA or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

Vaccines may be presented in unit-dose or multi-dose containers, such as sealed ampoules or vials. Such containers can be hermetically sealed to preserve sterility of the formulation until use. In general, formulations may be stored as suspensions, solutions or emulsions in oily or aqueous vehicles. Alternatively, a vaccine may be stored in a freeze-dried condition requiring only the addition of a sterile liquid carrier immediately prior to use. Vectors

Another aspect of the invention pertains to vectors containing a polynucleotide encoding a CPKPP or a portion thereof. One type of vector is a “plasmid,” which includes a circular double-stranded DNA loop into which additional DNA segments can be ligated. In the present specification, “plasmid” and “vector” can be used interchangeably as the plasmid is the most commonly used form of vector. Vectors include expression vectors and gene delivery vectors. The latter may be non-plasmid vectors such as viral vectors.

The expression vectors of the invention comprise a polynucleotide encoding a CPKPP or a portion thereof in a form suitable for expression of the polynucleotide in a host cell, which means that the expression vectors include one or more regulatory sequences, selected on the basis of the host cells to be used for expression, which are operatively linked to the polynucleotide sequence to be expressed. It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by polynucleotides as described herein (e.g., CPKPPs, mutant forms of CPKPPs, fusion proteins, and the like).

The expression vectors of the invention can be designed for expression of CPKPPs in prokaryotic or eukaryotic cells. For example, CPKPPs can be expressed in bacterial cells such as E. coli, insect cells (using baculovirus expression vectors), yeast cells, or mammalian cells. In certain embodiments, such protein may be used, for example, as a therapeutic protein of the invention. Alternatively, the expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of the recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia, Piscataway, N.J.), pMAL (New England Biolabs, Beverly, Mass.) and pRITS (Pharmacia, Piscataway, N.J.), which fuse GST, maltose E binding protein, or protein A, respectively, to the target recombinant protein.

Purified fusion proteins can be utilized in CPKPP activity assays, (e.g., direct assays or competitive assays described in detail below), or to generate antibodies specific for CPKPPs.

Examples of suitable inducible non-fusion E. coli expression vectors include pTrc and pET 11d. Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HSLE174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein. Another strategy is to alter the polynucleotide sequence of the polynucleotide to be inserted into an expression vector so that the individual codons for each amino acid are those preferentially utilized in E. coli. Such alteration of polynucleotide sequences of the invention can be carried out by standard DNA synthesis techniques.

In another embodiment, the CPKPP expression vector is a yeast expression vector. Examples of vectors for expression in yeast S. cerevisiae include pYepSec1, pMFa, pJRY88, pYES2 and picZ (Invitrogen Corp, San Diego, Calif.).

Alternatively, CPKPPs of the invention can be expressed in insect cells using baculovirus expression vectors. Baculovirus vectors available for expression of proteins in cultured insect cells (e.g., Sf9 cells) include the pAc series and the pVL series.

In yet another embodiment, a polynucleotide of the invention is expressed in mammalian cells using a mammalian expression vector. Examples of mammalian expression vectors include pCDM8 and pMT2PC. When used in mammalian cells, the expression vector's control functions are often provided by viral regulatory elements. For example, commonly used promoters are derived from polyoma, adenovirus 2, cytomegalovirus and Simian Virus 40. Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21 (DE3) or HSLE174(DE3) from a resident prophage harboring a T7 gn1 gene under the transcriptional control of the lacUV 5 promoter.

In another embodiment, the mammalian expression vector is capable of directing expression of the polynucleotide preferentially in a particular cell type (e.g., tissue-specific regulatory elements are used to express the polynucleotide). Tissue-specific regulatory elements are known in the art and may include epithelial cell-specific promoters. Other non-limiting examples of suitable tissue-specific promoters include the liver-specific albumin promoter, lymphoid-specific promoters, promoters of T cell receptors and immunoglobulins, neuron-specific promoters (e.g., the neurofilament promoter), pancreas-specific promoters, and mammary gland-specific promoters (e.g., milk whey promoter). Developmentally-regulated promoters are also encompassed, for example the α-fetoprotein promoter.

The CPKGs identified in the present invention can be used for therapeutical purposes. For example, antisense constructs of the CPKGs can be delivered therapeutically to cancer cells. The goal of such therapy is to retard the growth rate of the cancer cells. Expression of the sense molecules and their translation products or expression of the antisense mRNA molecules has the effect of inhibiting the growth rate of cancer cells or inducing apoptosis (a radical reduction in the growth rate of a cell).

The invention provides a recombinant expression vector comprising a polynucleotide encoding a CPKPP cloned into the expression vector in an antisense orientation. That is, the DNA molecule is operatively linked to a regulatory sequence in a manner which allows for expression (by transcription of the DNA molecule) of an RNA molecule which is antisense to mRNA corresponding to a CPKG of the invention. Regulatory sequences operatively linked to a polynucleotide cloned in the antisense orientation can be chosen to direct the continuous expression of the antisense RNA molecule in a variety of cell types. For instance viral promoters and/or enhancers, or regulatory sequences can be chosen to direct constitutive, tissue specific or cell type specific expression of antisense RNA. The antisense expression vector can be in the form of a recombinant plasmid, phagemid or attenuated virus in which antisense polynucleotides are produced under the control of a high efficiency regulatory region. The activity of the promoter/enhancer can be determined by the cell type into which the vector is introduced.

The invention further provides gene delivery vehicles for delivery of polynucleotides to cells, tissues, or a mammal for expression. For example, a polynucleotide sequence of the invention can be administered either locally or systemically in a gene delivery vehicle. These constructs can utilize viral or non-viral vector approaches in in vivo or ex vivo modality. Expression of such coding sequence can be induced using endogenous mammalian or heterologous promoters. Expression of the coding sequence in vivo can be either constituted or regulated. The invention includes gene delivery vehicles capable of expressing the contemplated polynucleotides. The gene delivery vehicle can be, without limitation, a viral vector, such as a retroviral, lentiviral, adenoviral, adeno-associated viral (AAV), herpes viral, or alphavirus vector. The viral vector can also be an astrovirus, coronavirus, orthomyxovirus, papovavirus, paramyxovirus, parvovirus, picomavirus, poxvirus, or togavirus viral vector.

Delivery of the gene therapy constructs of this invention into cells is not limited to the above mentioned viral vectors. Other delivery methods and media may be employed such as, for example, nucleic acid expression vectors, polycationic condensed DNA linked or unlinked to killed adenovirus alone, ligand linked DNA, liposome-DNA complex, eukaryotic cell delivery vehicles cells, deposition of photopolymerized hydrogel materials, handheld gene transfer particle gun, ionizing radiation, nucleic charge neutralization or fusion with cell membranes. Particle mediated gene transfer may be employed. Briefly, the sequence can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose or transferrin. Naked DNA may also be employed. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Regulatable Expression Systems

Another aspect of the invention pertains to the expression of CPKGs using a regulatable expression system. Systems to regulate expression of therapeutic genes have been developed and incorporated into the current viral and nonviral gene delivery vectors. Examples of these systems include, but are not limited to, Tet-on/off system, Ecdysone system, Progesterone-system, and Rapamycin-system.

Host Cells

Another aspect of the invention pertains to host cells into which a polynucleotide molecule of the invention is introduced, e.g., a CPKG listed in Table 1, or homolog thereof, within an expression vector, a gene delivery vector, or a polynucleotide molecule of the invention containing sequences which allow it to homologously recombine into a specific site of the host cell's genome. The terms “host cell” and “recombinant host cell” are used interchangeably and do not not only refer to a particular subject cell but also to the progeny or potential progeny of such cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a CPKPP of the invention can be expressed in bacterial cells such as E. coli, insect cells, yeast or mammalian cells (such as Chinese hamster ovary cells (CHO), COS cells, Fischer 344 rat cells, HLA-B27 rat cells, HeLa cells, A549 cells, or 293 cells). Other suitable host cells are known to those skilled in the art.

Vector DNA can be introduced into prokaryotic or eukaryotic cells via conventional transformation or transfection techniques. A variety of art-recognized techniques are available for introducing foreign polynucleotide (e.g., DNA) into a host cell, including calcium phosphate or calcium chloride co-precipitation, DEAE-dextran-mediated transfection, lipofection, or electoporation.

For stable transfection of mammalian cells, it is known that, depending upon the expression vector and transfection technique used, only a small fraction of cells may integrate the foreign DNA into their genome. In order to identify and select these integrants, a gene that encodes a selectable flag (e.g., resistance to antibiotics) is generally introduced into the host cells along with the gene of interest. Examples of selectable flags include those which confer resistance to drugs, such as G418, hygromycin and methotrexate. Polynucleotide encoding a selectable flag can be introduced into a host cell on the same vector as that encoding a CPKPP or can be introduced on a separate vector. Cells stably transfected with the introduced polynucleotide can be identified by drug selection (e.g., cells that have incorporated the selectable flag gene will survive, while the other cells die).

A host cell of the invention, such as a prokaryotic or eukaryotic host cell in culture, can be used to produce (i.e., express) a CPKPP. Accordingly, the invention further provides methods for producing a CPKPP using the host cells of the invention. In one embodiment, the method comprises culturing the host cell of invention (into which a recombinant expression vector encoding a CPKPP has been introduced) in a suitable medium such that a CPKPP of the invention is produced. In another embodiment, the method further comprises isolating a CPKPP from the medium or the host cell.

Transgenic and Knockout Animals

The host cells of the invention can also be used to produce non-human transgenic animals. For example, in one embodiment, a host cell of the invention is a fertilized oocyte or an embryonic stem cell into which CPKPP-coding sequences have been introduced. Such host cells can then be used to create non-human transgenic animals in which exogenous sequences encoding a CPKPP of the invention have been introduced into their genome or homologous recombinant animals in which endogenous sequences encoding the CPKPP of the invention have been altered. Such animals are useful for studying the function and/or activity of a CPKPP and for identifying and/or evaluating modulators of CPKPP activity.

A transgenic animal of the invention can be created by introducing a CPKPP-encoding polynucleotide into the mate pronuclei of a fertilized oocyte, e.g., by microinjection or retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to a transgene to direct expression of a CPKPP to particular cells. Methods for generating transgenic animals via embryo manipulation and microinjection, particularly animals such as mice, have become conventional in the art. Similar methods are used for production of other transgenic animals. A transgenic founder animal can be identified based upon the presence of a transgene of the invention in its genome and/or expression of mRNA corresponding to a gene of the invention in tissues or cells of the animals. A transgenic founder animal can then be used to breed additional animals carrying the transgene. Moreover, transgenic animals carrying a transgene encoding a CPKPP can further be bred to other transgenic animals carrying other transgenes.

To create a homologous recombinant animal (knockout animal), a vector is prepared which contains at least a portion of a gene of the invention into which a deletion, addition or substitution has been introduced to thereby alter, e.g., functionally disrupt, the gene. The gene can be a human gene or a non-human homolog of a human gene of the invention (e.g., a homolog of a CPKG listed in Table 1). For example, a mouse gene can be used to construct a homologous recombination polynucleotide molecule, e.g., a vector, suitable for altering an endogenous gene of the invention in the mouse genome. In one embodiment, the homologous recombination polynucleotide molecule is designed such that, upon homologous recombination, the endogenous gene of the invention is functionally disrupted (i.e., no longer encodes a functional protein; also referred to as a “knockout” vector). Alternatively, the homologous recombination polynucleotide molecule can be designed such that, upon homologous recombination, the endogenous gene is mutated or otherwise altered but still encodes functional protein (e.g., the upstream regulatory region can be altered to thereby alter the expression of the endogenous CPKPP). In the homologous recombination polynucleotide molecule, the altered portion of the gene of the invention is flanked at its 5′ and 3′ ends by additional polynucleotide sequence of the gene of the invention to allow for homologous recombination to occur between the exogenous gene carried by the homologous recombination polynucleotide molecule and an endogenous gene in a cell, e.g., an embryonic stem cell. The additional flanking polynucleotide sequence is of sufficient length for successful homologous recombination with the endogenous gene.

Typically, several kilobases of flanking DNA (both at the 5′ and 3′ ends) are included in the homologous recombination polynucleotide molecule. The homologous recombination polynucleotide molecule is introduced into a cell, e.g., an embryonic stem cell line (e.g., by electroporation) and cells in which the introduced gene has homologously recombined with the endogenous gene are selected. The selected cells can then be injected into a blastocyst of an animal (e.g., a mouse) to form aggregation chimeras. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination polynucleotide molecules is well-known in the art.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Laksa et al., Proc. Natl. Acad. Sci., USA, 89:6232-6236, 1992. Another example of a recombinase system is the FLP recombinase system of Saccharomyces cerevisiae (O'Gorman et al., Science, 251:1351-1355, 1991). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al., Nature, 385:810-813, 1997, and PCT International Publication Nos. WO97/07668 and WO97/07669. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

In many embodiments of the invention, the non-human transgenic animals comprise a CPKG, such as, for example, STK15. In some other embodiments, the non-human “knock-out” animal is a STK15 knock-out.

Detection Methods

As discussed earlier, expression level of CPKGs may be used as a marker for cancer. Detection and measurement of the relative amount of a CPKG product (polynucleotide or polypeptide) of the invention can be by any method known in the art.

Methodologies for detection of a transcribed polynucleotide include RNA extraction from a cell or tissue sample, followed by hybridization of a labeled probe (i.e., a complementary polynucleotide molecule) specific for the target RNA to the extracted RNA and detection of the probe (i.e., Northern blotting).

Methodologies for peptide detection include protein extraction from a cell or tissue sample, followed by binding of an antibody specific for the target protein to the protein sample, and detection of the antibody. For example, detection of STK15 may be accomplished using polyclonal anti-STK15 antibody. Antibodies are generally detected by the use of a labeled secondary antibody. The label can be a radioisotope, a fluorescent compound, an enzyme, an enzyme co-factor, or ligand. Such methods are well understood in the art.

In certain embodiments, the CPKGs themselves (i.e., the DNA or cDNA) may serve as markers for cancer. For example, an increase of genomic copies of a CPKG, such as by duplication of the gene, may also be correlated with cancer.

Detection of specific polynucleotide molecules may also be assessed by gel electrophoresis, column chromatography, or direct sequencing, quantitative PCR (in the case of polynucleotide molecules), RT-PCR, or nested-PCR among many other techniques well-known to those skilled in the art.

Detection of the presence or number of copies of all or a part of a CPKG of the invention may be performed using any method known in the art. It is convenient to assess the presence and/or quantity of a DNA or cDNA by Southern analysis, in which total DNA from a cell or tissue sample is extracted, is hybridized with a labeled probe (i.e., a complementary DNA molecules), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

In certain embodiments, the CPKPPs may serve as markers for cancer. Detection of specific polypeptide molecules may be assessed by gel electrophoresis, Western blot, column chromatography, or direct sequencing, among many other techniques well-known to those skilled in the art.

Panels of CPKGs

Expression level of each CPKG may be considered individually, although it is within the scope of the invention to provide combinations of two or more CPKGs for use in the methods and compositions of the invention to increase the confidence of the analysis. In another aspect, the invention provides panels of the CPKGs of the invention. A panel of CPKGs comprises two or more CPKGs. A panel may also comprise 2-5,5-15, 15-35, 35-50, or more than 50 CPKGs. In one embodiment, these panels of CPKGs are selected such that the CPKGs within any one panel share certain features. For example, the CPKGs of a first panel may be protein kinases that exhibit at least a two-fold increase in quantity or activity in a cancer sample, as compared to a sample which is substantially free of cancer from the same subject or a sample which is substantially free of cancer from a different subject without cancer. Alternatively, CPKGs of a second panel may each exhibit differential regulation as compared to a first panel. Similarly, different panels of CPKGs may be composed of CPKGs representing different stages of cancer. Panels of the CPKGs of the invention may be made by independently selecting CPKGs from Table 1, and may further be provided on biochips, as discussed below.

Screening Methods

The invention also provides methods (also referred to herein as “screening assays”) for identifying modulators, i.e., candidate or test compounds or agents comprising therapeutic moieties (e.g., peptides, peptidomimetics, peptoids, polynucleotides, small molecules or other drugs) which (a) bind to a CPKPP, or (b) have a modulatory (e.g., up-regulation or down-regulation; stimulatory or inhibitory; potentiation/induction or suppression) effect on the activity of a CPKPP or, more specifically, (c) have a modulatory effect on the interactions of the CPKPP with one or more of its natural substrates, or (d) have a modulatory effect on the expression of the CPKPPs. Such assays typically comprise a reaction between the CPKPP and one or more assay components. The other components may be either the test compound itself, or a combination of test compound and a binding partner of the CPKPP.

The test compounds of the present invention are generally either small molecules or biomolecules. Small molecules include, but are not limited to, inorganic molecules and small organic molecules. Biomolecules include, but are not limited to, naturally-occurring and synthetic compounds that have a bioactivity in mammals, such as polypeptides, polysaccharides, and polynucleotides. In one embodiment the test compound is a small molecule. In another embodiment, the test compound is a biomolecule. One skilled in the art will appreciate that the nature of the test compound may vary depending on the nature of the protein encoded by the CPKG of the invention. For example, if the CPKG encodes an orphan receptor having an unknown ligand, the test compound may be any of a number of biomolecules which may act as cognate ligand, including but not limited to, cytokines, lipid-derived mediators, small biogenic amines, hormones, neuropeptides, or proteases.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., J. Med. Chem., 37:2678-85, 1994); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer Drug Des., 12:145, 1997).

Screening for Inhibitors of CPKPP

The invention provides methods of screening test compounds for inhibitors of CPKPP, and to the pharmaceutical compositions comprising the test compounds. The method of screening comprises obtaining samples from subjects diagnosed with or suspected of having cancer, contacting each separate aliquot of the samples with one of a plurality of test compounds, and comparing expression of one or more CPKGs in each of the aliquots to determine whether any of the test compounds provides a substantially decreased level of expression or activity of a CPKG relative to samples with other test compounds or relative to an untreated sample or control sample. In addition, methods of screening may be devised by combining a test compound with a protein and thereby determining the effect of the test compound on the protein.

In addition, the invention is further directed to a method of screening for test compounds capable of modulating with the binding of a CPKPP and a binding partner, by combining the test compound, CPKPP, and binding partner together and determining whether binding of the binding partner and CPKPP occurs. The test compound may be either small molecules or a biomolecule. As discussed below, test compounds may be provided from a variety of libraries well-known in the art.

Modulators of a CPKG expression, activity or binding ability are useful as therapeutic compositions of the invention. Such modulators (e.g., antagonists or agonists) may be formulated as pharmaceutical compositions, as described herein below. Such modulators may also be used in the methods of the invention, for example, to diagnose, treat, or prognose cancer.

High-Throughput Screening Assays

The invention provides methods of conducting high-throughput screening for test compounds capable of inhibiting activity or expression of a CPKPP of the present invention. In one embodiment, the method of high-throughput screening involves combining test compounds and the CPKPP and detecting the effect of the test compound on the CPKPP.

A variety of high-throughput functional assays well-known in the art may be used in combination to screen and/or study the reactivity of different types of activating test compounds. Since the coupling system is often difficult to predict, a number of assays may need to be configured to detect a wide range of coupling mechanisms. A variety of fluorescence-based techniques are well-known in the art and are capable of high-throughput and ultra high throughput screening for activity, including but not limited to BRET® or FRET® (both by Packard Instrument Co., Meriden, Conn.). The ability to screen a large volume and a variety of test compounds with great sensitivity permits for analysis of the therapeutic targets of the invention to further provide potential inhibitors of cancer. For example, where the CPKG encodes an orphan receptor with an unidentified ligand, high-throughput assays may be utilized to identify the ligand, and to further identify test compounds which prevent binding of the receptor to the ligand. The BIACORE® system may also be manipulated to detect binding of test compounds with individual components of the therapeutic target, to detect binding to either the encoded protein or to the ligand.

By combining test compounds with CPKPPs of the invention and determining the binding activity between such, diagnostic analysis can be performed to elucidate the coupling systems. Generic assays using cytosensor microphysiometer may also be used to measure metabolic activation, while changes in calcium mobilization can be detected by using the fluorescence-based techniques such as FLIPR® (Molecular Devices Corp, Sunnyvale, Calif.). In addition, the presence of apoptotic cells may be determined by TUNEL assay, which utilizes flow cytometry to detect free 3-OH termini resulting from cleavage of genomic DNA during apoptosis. As mentioned above, a variety of functional assays well-known in the art may be used in combination to screen and/or study the reactivity of different types of activating test compounds. In some cases, the high-throughput screening assay of the present invention utilizes label-free plasmon resonance technology as provided by BIACORE® systems (Biacore International AB, Uppsala, Sweden). Plasmon free resonance occurs when surface plasmon waves are excited at a metal/liquid interface. By reflecting directed light from the surface as a result of contact with a sample, the surface plasmon resonance causes a change in the refractive index at the surface layer. The refractive index change for a given change of mass concentration at the surface layer is similar for many bioactive agents (including proteins, peptides, lipids and polynucleotides), and since the BIACORE® sensor surface can be functionalized to bind a variety of these bioactive agents, detection of a wide selection of test compounds can thus be accomplished.

Therefore, the invention provides for high-throughput screening of test compounds for the ability to inhibit activity of a protein encoded by the CPKGs listed in Table 1, by combining the test compounds and the protein in high-throughput assays such as BIACORE®, or in fluorescence-based assays such as BRET®. In addition, high-throughput assays may be utilized to identify specific factors which bind to the encoded proteins, or alternatively, to identify test compounds which prevent binding of the receptor to the binding partner. In the case of orphan receptors, the binding partner may be the natural ligand for the receptor. Moreover, the high-throughput screening assays may be modified to determine whether test compounds can bind to either the encoded protein or to the binding partner (e.g., substrate or ligand) which binds to the protein.

In one embodiment, the high-throughput screening assay detects the ability of a plurality of test compounds to bind to a Group I gene product. In another specific embodiment, the high-throughput screening assay detects the ability of a plurality of a test compound to inhibit a binding partner (such as a ligand) to bind to a Group I gene product. In yet another specific embodiment, the high-throughput screening assay detects the ability of a plurality of a test compounds to modulate signaling through a Group I gene product.

Predictive Medicine

The present invention pertains to the field of predictive medicine in which diagnostic assays, prognostic assays, pharmacogenetics and monitoring clinical trials are used for prognostic (predictive) purpose to thereby treat an individual prophylactically. Accordingly, one aspect of the present invention relates to diagnostic assays for determining CPKG polynucleotide and/or polypeptide expression and/or activity, in the context of a biological sample (e.g., blood, serum, cells, tissue) to thereby determine whether an individual is at risk for developing cancer associated with modulated CPKG expression or activity. The invention also provides for prognostic (or predictive) assays for determining whether an individual is at risk of developing cancer associated with aberrant CPKG protein or polynucleotide expression or activity.

For example, the number of copies of a CPKG can be assayed in a biological sample. Such assays can be used for prognostic or predictive purposes to thereby prophylactically treat an individual prior to the onset of cancer associated with aberrant CPKG protein, polynucleotide expression or activity.

Another aspect of the invention pertains to monitoring the influence of agents (e.g., drugs, compounds) on the expression or activity of CPKGs in clinical trials.

Diagnostic Assays

An exemplary method for detecting the presence or absence of a CPKPP or polynucleotide encoding a CPKPP in a biological sample involves contacting a biological sample with a compound or an agent capable of detecting the CPKPP or polynucleotide (e.g., mRNA, genomic DNA) that encodes the CPKPP such that the presence of the CPKPP or polynucleotide is detected in the biological sample. One example agent for detecting mRNA or genomic DNA corresponding to a CPKG or CPKPP of the invention is a labeled polynucleotide probe capable of hybridizing to an mRNA or genomic DNA of the invention. In one embodiment, the polynucleotides to be screened are arranged on a GeneChip®. Suitable probes for use in the diagnostic assays of the invention are described herein. One example agent for detecting a CPKPP of the invention is an antibody which specifically recognizes the CPKPP.

The diagnostic assays may also be used to quantify the amount of expression or activity of a CPKG in a biological sample. Such quantification is useful, for example, to determine the progression or severity of cancer. Such quantification is also useful, for example, to determine the severity of cancer following treatment.

Determining Severity of Cancer

In the field of diagnostic assays, the invention also provides methods for determining the severity of cancer by isolating a sample from a subject (e.g., a biopsy), detecting the presence, quantity and/or activity of one or more CPKGs of the invention in the sample relative to a second sample from a normal sample or control sample. In one embodiment, the expression levels of CPKGs in the two samples are compared, and a modulation in one or more CPKGs in the test sample indicates cancer. In other embodiments the modulation of 2, 3, 4 or more CPKGs indicate a severe case of cancer.

In another aspect, the invention provides CPKGs whose quantity or activity is correlated with the severity of cancer. The subsequent level of expression may further be compared to different expression profiles of various stages of the cancer to confirm whether the subject has a matching profile. In yet another aspect, the invention provides CPKGs whose quantity or activity is correlated with a risk in a subject for developing cancer.

In one embodiment, the agent for detecting CPKPP is an antibody capable of binding to CPKPP, including an antibody with a detectable label. Antibodies can be, for example, polyclonal or monoclonal. An intact antibody, or a fragment thereof (e.g., Fab or F(ab′)₂) can be used. The probe or antibody can be directly labeled by coupling (i.e., physically linking) a detectable substance to the probe or antibody and can be indirectly labeled by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. Biological sample includes tissues, cells and biological fluids isolated from a subject, as well as tissues, cells and fluids present within a subject. That is, the detection method of the invention can be used to detect CPKG mRNA, protein or genomic DNA in a biological sample in vitro as well as in vivo. For example, in vitro techniques for detection of CPKG mRNA include Northern hybridizations and in situ hybridizations. In vitro techniques for detection of CPKPP include enzyme linked immunosorbent assays (ELISAs), Western blots, immunoprecipitations and immunofluorescence. In vitro techniques for detection of CPKG genomic DNA include Southern hybridizations. Furthermore, in vivo techniques for detection of CPKPP include introducing into a subject a labeled anti-CPKPP antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. An exemplary biological sample is a serum sample isolated by conventional means from a subject, e.g., a biopsy or blood draw.

In another embodiment, the methods further involve obtaining a control biological sample from a subject, contacting the control sample with a compound or agent capable of detecting CPKG protein, mRNA, or genomic DNA of, such that the presence of CPKG protein, mRNA or genomic DNA is detected in the biological sample, and comparing the presence of CPKG protein, mRNA or genomic DNA in the control sample with the presence of CPKG protein, mRNA or genomic DNA in the test sample.

Detection of CPKPP Specific T cells

Cancer may also be detected based on the presence of T cells that specifically react with a CPKPP in a biological sample. Within certain methods, a biological sample comprising CD4⁺ and/or CD8⁺ T cells isolated from a patient is incubated with a CPKPP, a polynucleotide encoding such a polypeptide and/or an APC that expresses at least an immunogenic portion of such a polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with polypeptide (e.g., 5-25 μg/ml). It may be desirable to incubate another aliquot of a T cell sample in the absence of tumor polypeptide to serve as a control. For CD4⁺ T cells, activation can be detected, for instance, by evaluating proliferation of the T cells. For CD8⁺ T cells, activation can be detected, for instance, by evaluating cytolytic activity. A level of proliferation that is at least two-fold greater and/or a level of cytolytic activity that is at least 20% greater than in disease-free patients indicates the presence of cancer in the patient.

Prognostic Assays

The diagnostic method described herein can furthermore be utilized to identify subjects having or at risk of developing colon cancer associated with aberrant CPKG expression or activity.

The assays described herein, such as the preceding or following assays, can be utilized to identify a subject having cancer associated with an aberrant level of CPKG activity or expression. Alternatively, the prognostic assays can be utilized to identify a subject at risk for developing cancer associated with aberrant levels of CPKG protein activity or polynucleotide expression. Thus, the present invention provides a method for identifying cancer associated with aberrant CPKG expression or activity in which a test sample is obtained from a subject and CPKG protein or polynucleotide (e.g., mRNA or genomic DNA) is detected, wherein the presence of CPKG protein or polynucleotide is diagnostic or prognostic for a subject having or at risk of developing cancer with aberrant CPKG expression or activity.

Furthermore, the prognostic assays described herein can be used to determine whether a subject can be administered an agent (e.g., an agonist, antagonist, peptidomimetic, protein, peptide, polynucleotide, small molecule, or other drug candidate) to treat or prevent cancer associated with aberrant CPKG expression or activity, such as, for example, a cytokine. For example, such methods can be used to determine whether a subject can be effectively treated with an agent to inhibit cancer. Thus, the present invention provides methods for determining whether a subject can be effectively treated with an agent for cancer associated with increased CPKG expression or activity in which a test sample is obtained and CPKG protein or polynucleotide expression or activity is detected (e.g., wherein the abundance of CPKG protein or polynucleotide expression or activity is diagnostic for a subject that can be administered the agent to treat injury associated with aberrant CPKG expression or activity).

Prognostic assays can be devised to determine whether a subject undergoing treatment for cancer has a poor outlook for long term survival or disease progression. In one embodiment, prognosis can be determined shortly after diagnosis, i.e., within a few days. By establishing expression profiles of different stages of CPKGs, from onset to later stages, an expression pattern may emerge to correlate a particular expression profile to increased likelihood of a poor prognosis. The prognosis may then be used to devise a more aggressive treatment program and enhance the likelihood of long-term survival and well-being.

The methods of the invention can also be used to detect genetic alterations in a CPKG, thereby determining if a subject with the altered gene is at risk for damage characterized by aberrant regulation in CPKG protein activity or polynucleotide expression. In some embodiments, the methods include detecting, in a sample of cells from the subject, the presence or absence of a genetic alteration characterized by at least one alteration affecting the integrity of a CPKG, or the aberrant expression of the CPKG. For example, such genetic alterations can be detected by ascertaining the existence of at least one of the following: 1) deletion of one or more nucleotides from a CPKG; 2) addition of one or more nucleotides to a CPKG; 3) substitution of one or more nucleotides of a CPKG; 4) a chromosomal rearrangement of a CPKG; 5) alteration in the level of a messenger RNA transcript of a CPKG; 6) aberrant modification of a CPKG, such as of the methylation pattern of the genomic DNA; 7) the presence of a non-wild-type splicing pattern of a messenger RNA transcript of a CPKG; 8) non-wild-type level of a CPKG protein; 9) allelic loss of a CPKG; and 10) inappropriate post-translational modification of a CPKG protein. As described herein, there are a large number of assays known in the art which can be used for detecting alterations in a CPKG. An exemplary biological sample is a blood sample isolated by conventional means from a subject.

In certain embodiments, detection of the alteration involves the use of a probe/primer in a polymerase chain reaction (PCR), such as anchor PCR or RACE PCR, or, alternatively, in a ligation chain reaction (LCR), the latter of which can be particularly useful for detecting point mutations in the CPKG. This method can include the steps of collecting a cell sample of from a subject, isolating a polynucleotide sample (e.g., genomic, mRNA or both) from the cell sample, contacting the polynucleotide sample with one or more primers which specifically hybridize to a CPKG under conditions such that hybridization and amplification of the CPKG (if present) occur, and detecting the presence or absence of an amplification product, or detecting the size of the amplification product and comparing the length to a control sample. It is understood that PCR and/or LCR may be desirable to use as a preliminary amplification step in conjunction with any of the techniques used for detecting mutations described herein.

Alternative amplification methods include: self sustained sequence replication, transcriptional amplification system, Q-Beta Replicase, or any other polynucleotide amplification method, followed by the detection of the amplified molecules using techniques well-known to those of skill in the art. These detection schemes are especially useful for the detection of polynucleotide molecules if such molecules are present in very low numbers.

In an alternative embodiment, mutations in a CPKG from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicate mutations in the sample DNA. Moreover, sequence specific ribozymes (see, for example, U.S. Pat. No. 5,498,531) can be used to score for the presence of specific mutations by development or loss of a.ribozyme cleavage site.

In other embodiments, genetic mutations in a CPKG can be identified by hybridizing sample and control polynucleotides, e.g., DNA or RNA, to high density arrays containing hundreds or thousands of oligonucleotide probes. For example, genetic mutations in a CPKG can be identified in two dimensional arrays containing light generated DNA probes. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the CPKG and detect mutations by comparing the sequence of the sample CPKG with the corresponding wild-type (control) sequence. It is also contemplated that any of a variety of automated sequencing procedures can be utilized when performing the diagnostic assays, including sequencing by mass spectrometry.

Other methods for detecting mutations in a CPKG include methods in which protection from cleavage agents is used to detect mismatched bases in RNA/RNA or RNA/DNA heteroduplexes. In general, the art technique of “mismatch cleavage” starts by providing heteroduplexes by hybridizing (labeled) RNA or DNA containing the wild-type CPKG sequence with potentially mutant RNA or DNA obtained from a tissue sample. The double-stranded duplexes are treated with an agent which cleaves single-stranded regions of the duplex, which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. In one embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in CPKG cDNAs obtained from samples of cells. For example, the mutY enzyme of E. coli cleaves A at G/A mismatches and the thymidine DNA glycosylase from HeLa cells cleaves T at G/T mismatches. According to another embodiment, a probe based on a CPKG sequence, e.g., a wild-type CPKG sequence, is hybridized to cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in CPKGs. For example, single strand conformation polymorphism (SSCP) may be used to detect differences in electrophoretic mobility between mutant and wild-type polynucleotides. Single-stranded DNA fragments of sample and control CPKG polynucleotides will be denatured and allowed to renature. The secondary structure of single-stranded polynucleotides varies according to sequence. The resulting alteration in electrophoretic mobility enables the detection of even a single base change. The DNA fragments may be labeled or detected with labeled probes. The sensitivity of the assay may be enhanced by using RNA (rather than DNA) in which the secondary structure is more sensitive to a change in sequence. In one embodiment, the subject method utilizes heteroduplex analysis to separate double-stranded heteroduplex molecules on the basis of changes in electrophoretic mobility (Keen et al., Trends Genet., 7:5, 1991).

In yet another embodiment the movement of mutant or wild-type fragments in polyacrylamide gels containing a gradient of denaturant is assayed using denaturing gradient gel electrophoresis (DGGE). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner, Biophys. Chem., 265:12753, 1987).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al., Proc. Natl. Acad. Sci., USA, 86:6230, 1989). Such allele specific oligonucleotides are hybridized to PCR amplified target or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) or at the extreme 3′ end of one primer where, under appropriate conditions, mismatch can prevent or reduce polymerase extension. In addition, it may be desirable to introduce a novel restriction site in the region of the mutation to create cleavage-based detection. It is anticipated that, in certain embodiments, amplification may also be performed using Taq ligase for amplification. In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing prepackaged diagnostic kits comprising at least one probe polynucleotide or antibody reagent described herein, which may be conveniently used, e.g., in clinical settings to diagnose subjects exhibiting symptoms or family history of a disease or illness involving a CPKG.

Furthermore, any cell type or tissue in which a CPKG is expressed may be utilized in the prognostic or diagnostic assays described herein.

Monitoring Effects During Clinical Trials

Monitoring the influence of agents (e.g., drugs, small molecules and biomolecule) on the expression or activity of a CPKG protein can be applied not only in basic drug screening, but also in clinical trials. For example, the effectiveness of an agent determined by a screening assay, as described herein to decrease CPKG expression, protein levels, or downregulate CPKG activity, can be monitored in clinical trials of subjects exhibiting increased CPKG expression, protein levels, or up-regulated CPKG activity. In such clinical trials, the expression or activity of a CPKG can be used as a “read out” of the phenotype of a particular tissue.

For example, and not by way of limitation, genes, including CPKGs, that are modulated in tissues by treatment with an agent that modulates CPKPP activity (e.g., identified in a screening assay as described herein) can be identified. Thus, to study the effect of agents on CPKPP-associated damage, for example, in a clinical trial, cells can be isolated and RNA prepared and analyzed for the levels of expression of a CPKG. The levels of gene expression or a gene expression pattern can be quantified by Northern blot analysis, RT-PCR or GeneChip® as described herein, or alternatively by measuring the amount of protein produced, by one of the methods as described herein, or by measuring the levels of activity of CPKPP. In this way, the gene expression pattern can serve as a read-out, indicative of the physiological response of the cells to the agent. Accordingly, this response state may be determined before treatment and at various points during treatment of the individual with the agent.

In one embodiment, the present invention provides a method for monitoring the effectiveness of treatment of a subject with an agent (e.g., an agonist, antagonist, peptidomimetic, biomolecule, small molecule, or other drug candidate identified by the screening assays described herein) including the steps of (i) obtaining a pre-administration sample from a subject prior to administration of the agent; (ii) detecting the level of expression of a CPKG protein or mRNA in the pre-administration sample; (iii) obtaining one or more post-administration samples from the subject; (iv) detecting the level of expression or activity of the CPKG protein or mRNA in the post-administration samples; (v) comparing the level of expression or activity of the CPKG protein or mRNA in the pre-administration sample with the CPKG protein or mRNA the post administration sample or samples; and (vi) altering the administration of the agent to the subject accordingly. For example, decreased administration of the agent may be desirable to decrease expression or activity of CPKG to lower levels than detected, i.e., to decrease the effectiveness of the agent. According to such an embodiment, CPKG expression or activity may be used as an indicator of the effectiveness of an agent, even in the absence of an observable phenotypic response.

Methods of Treatment

The present invention provides for both prophylactic and therapeutic methods of treating a subject at risk for, susceptible to or diagnosed with cancer. With regard to both prophylactic and therapeutic methods of treatment, such treatments may be specifically tailored or modified, based on knowledge obtained from the field of pharmacogenomics. Pharmacogenomics includes the application of genomics technologies such as gene sequencing, statistical genetics, and gene expression analysis to drugs in clinical development and on the market and the study of how a subject's genes determine his or her response to a drug (e.g., a subject's “drug response phenotype” or “drug response genotype”). Thus, another aspect of the invention provides methods for tailoring an individual's prophylactic or therapeutic treatment with either the CPKPP molecules of the present invention or CPKPP modulators (e.g., agonists or antagonists) according to that individual's drug response. Pharmacogenomics allows a clinician or physician to target prophylactic or therapeutic treatments to subjects who will most benefit from the treatment and to avoid treatment of subjects who will experience toxic drug-related side effects.

Prophylactic Methods

In one aspect, the invention provides a method for preventing in a subject cancer associated with aberrant CPKG expression or activity, by administering to the subject a CPKG protein or an agent which modulates CPKG protein expression or activity.

Subjects at risk for cancer which is caused or contributed to by aberrant CPKG expression or activity can be identified by, for example, any or a combination of diagnostic or prognostic assays as described herein.

Administration of a prophylactic agent can occur prior to the manifestation of symptoms characteristic of the differential CPKG protein expression, such that cancer is prevented or, alternatively, delayed in its progression. Depending on the type of CPKG aberrancy (e.g., typically a modulation outside the normal standard deviation), a CPKG protein, CPKG agonist or antagonist agent can be used for treating the subject. The appropriate agent can be determined based on screening assays described herein.

Therapeutic Methods

Another aspect of the invention pertains to methods of modulating CPKG protein expression or activity for therapeutic purposes. Accordingly, in one embodiment, the modulatory method of the invention involves contacting a cell with an agent that modulates one or more of the activities of a CPKG product activity associated with the cell. An agent that modulates CPKG product activity can be an agent as described herein, such as a polynucleotide (e.g., an antisense molecule) or a polypeptide (e.g., a dominant-negative mutant of a CPKPP), a naturally-occurring target molecule of a CPKPP (e.g., a CPKPP substrate), an anti-CPKPP antibody, a CPKPP modulator (e.g., agonist or antagonist), a peptidomimetic of a CPKG protein agonist or antagonist, or other small molecules.

The invention further provides methods of modulating a level of expression of a CPKG of the invention, comprising administration to a subject having cancer, a variety of compositions which correspond to the CPKGs of Table 1, including proteins or antisense oligonucleotides. The protein may be provided by further providing a vector comprising a polynucleotide encoding the protein to the cells. Alternatively, the expression levels of the CPKGs of the invention may be modulated by providing an antibody, a plurality of antibodies or an antibody conjugated to a therapeutic moiety. Treatment with the antibody may further be localized to the tissue comprising cancer. In another aspect, the invention provides methods for localizing a therapeutic moiety to cancer tissue or cells comprising exposing the tissue or cells to an antibody which is specific to a protein encoded by the CPKGs of the invention. This method may therefore provide a means to inhibit expression of a specific gene corresponding to a CPKG listed in Table 1.

Determining Efficacy of a Test Compound or Therapy

The invention also provides methods of assessing the efficacy of a test compound or therapy for inhibiting cancer in a subject. These methods involve isolating samples from a subject suffering from cancer, who is undergoing treatment or therapy, and detecting the presence, quantity, and/or activity of one or more CPKGs of the invention in the first sample relative to a second sample. Where the efficacy of a test compound is determined, the first and second samples can be, for example, sub-portions of a single sample taken from the subject, wherein the first portion is exposed to the test compound and the second portion is not. In one aspect of this embodiment, the CPKG is expressed at a substantially decreased level in the first sample, relative to the second. In some instances, the level of expression in the first sample approximates (i.e., is less than the standard deviation for normal samples) the level of expression in a third control sample, taken from a control sample of normal tissue. This result suggests that the test compound inhibits the expression of the CPKG in the sample. In another aspect of this embodiment, the CPKG is expressed at a substantially increased level in the first sample, relative to the second. In some other instances, the level of expression in the first sample approximates (i.e., is less than the standard deviation for normal samples) the level of expression in a third control sample, taken from a control sample of normal tissue. This result suggests that the test compound augments the expression of the CPKG in the sample.

Where the efficacy of a therapy is being assessed, the first sample obtained from the subject can be obtained prior to provision of at least a portion of the therapy, whereas the second sample is obtained following provision of the portion of the therapy. The levels of CPKGs in the samples are compared, for example, against a third control sample as well, and correlated with the presence, or risk of presence, of cancer. In one embodiment, the level of CPKGs in the second sample approximates the level of expression of a third control sample. In the present invention, a substantially decreased level of expression of a CPKG indicates that the therapy is efficacious for treating cancer.

Pharmacogenomics

The CPKG protein and polynucleotide molecules of the present invention, as well as agents, inhibitors or modulators which have a stimulatory or inhibitory effect on CPKG or CPKG protein as identified by a screening assay described herein, can be administered to individuals to treat (prophylactically or therapeutically) cancer associated with aberrant CPKG activity.

In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a CPKG product (polynucleotide or polypeptide) or CPKG modulator as well as tailoring the dosage and/or therapeutic regimen of treatment with a CPKG product or CPKG modulator.

Pharmacogenomics deals with clinically significant hereditary variations in the response to drugs due to altered drug disposition and abnormal action in affected persons. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as a “genome-wide association,” relies primarily on a high-resolution map of the human genome consisting of already known gene-related sites (e.g., a “bi-allelic” gene marker map which consists of 60,000-100,000 polymorphic or variable sites on the human genome, each of which has two variants). Such a high-resolution genetic map can be compared to a map of the genome of each of a statistically substantial number of subjects taking part in a Phase II/III drug trial to identify genes associated with a particular observed drug response or side effect. Alternatively, such a high resolution map can be generated from a combination of some ten-million known single nucleotide polymorphisms (SNPs) in the human genome. A SNP may be involved in a disease process. However, the vast majority of SNPs may not be disease associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals. Thus, mapping of the CPKGs of the invention to SNP maps of cancer patients may allow easier identification of these genes according to the genetic methods described herein.

Alternatively, a method termed the “candidate gene approach,” can be utilized to identify genes that predict drug response. According to this method, if a gene that encodes a drug target is known (e.g., a CPKG of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYPZC19) has provided an explanation as to why some subjects do not obtain the expected drug effects or show exaggerated drug response and serious toxicity after taking the standard and safe dose of a drug. These polymorphisms are expressed in two phenotypes in the population, the extensive metabolizer and poor metabolizer. The prevalence of poor metabolizer phenotypes is different among different populations. For example, the gene coding for CYP2D6 is highly polymorphic and several mutations have been identified in poor metabolizers, which all lead to the absence of functional CYP2D6. Poor metabolizers of CYP2D6 and CYP2C19 quite frequently experience exaggerated drug response and side effects when they receive standard doses. If a metabolite is the active therapeutic moiety, poor metabolizers show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., CPKG expression in response to a CPKG modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a CPKG product or CPKG modulator, such as a modulator identified by one of the exemplary screening assays described herein.

Pharmaceutical Compositions

The invention is further directed to pharmaceutical compositions comprising the test compound, or bioactive agent, or a CPKG modulator (i.e., agonist or antagonist), which may further include a CPKG product, and can be formulated as described herein. Alternatively, these compositions may include an antibody which specifically binds to a CPKG protein of the invention and/or an antisense polynucleotide molecule which is complementary to a CPKG polynucleotide of the invention and can be formulated as described herein.

One or more of the CPKGs of the invention, fragments of CPKGs, CPKG products, fragments of CPKG products, CPKG modulators, or anti-CPKPP antibodies of the invention can be incorporated into pharmaceutical compositions suitable for administration.

Suitable pharmaceutically acceptable carriers include solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

The invention includes methods for preparing pharmaceutical compositions for modulating the expression or activity of a polypeptide or polynucleotide corresponding to a CPKG of the invention. Such methods comprise formulating a pharmaceutically acceptable carrier with an agent which modulates expression or activity of a polypeptide or polynucleotide corresponding to a CPKG of the invention. Such compositions can further include additional active agents. Thus, the invention further includes methods for preparing a pharmaceutical composition by formulating a pharmaceutically acceptable carrier with an agent which modulates expression or activity of a polypeptide or polynucleotide corresponding to a CPKG of the invention and one or more additional bioactive agents.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols such as manitol, sorbitol, sodium chloride can be included in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a CPKPP or an anti-CPKPP antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, examples of methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bioactive compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from e.g. Alza Corporation and Nova Pharmaceuticals, Inc. Liposomal suspensions (including liposomes targeted to infected cells with monoclonal antibodies to viral antigens) can also be used as pharmaceutically acceptable carriers.

It is especially advantageous to formulate oral or parenteral compositions in dosage unit form for ease of administration and uniformity of dosage. Dosage unit form as used herein includes physically discrete units suited as unitary dosages for the subject to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In many embodiments, compounds which exhibit large therapeutic indices are selected. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds can lie within a range of circulating concentrations that includes the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The CPKGs of the invention can be inserted into gene delivery vectors and used as gene therapy vectors. Gene therapy vectors can be delivered to a subject by, for example, intravenous administration, intraportal administration, intrabiliary administration, intra-arterial administration, direct injection into the liver parenchyma, by intramusclular injection, by inhalation, by perfusion, or by stereotactic injection. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Kits

The invention also encompasses kits for detecting the presence of a CPKG product in a biological sample, the kit comprising reagents for assessing expression of the CPKGs of the invention. The reagents may be an antibody or fragment thereof, wherein the antibody or fragment thereof specifically binds with a protein corresponding to a CPKG from Table 1. For example, antibodies of interest may be prepared by methods known in the art. Optionally, the kits may comprise a polynucleotide probe wherein the probe specifically binds with a transcribed polynucleotide corresponding to a CPKG. The kits may also include an array of CPKGs arranged on a biochip, such as, for example, a GeneChip®. The kit may contain means for determining the amount of the CPKG protein or mRNA in the sample and means for comparing the amount of the CPKG protein or mRNA in the sample with a control or standard. The compound or agent can be packaged in a suitable container. The kit can further comprise instructions for using the kit to detect CPKG protein or polynucleotide

The invention further provides kits for assessing the suitability of each of a plurality of compounds for inhibiting cancer in a subject. Such kits include a plurality of compounds to be tested, and a reagent (i.e., antibody specific to corresponding proteins, or a probe or primer specific to corresponding polynucleotides) for assessing expression of a CPKG listed in Table 1.

Computer Readable Means and Arrays

Computer readable media comprising CPKG information of the present invention is also provided. Suitable computer readable media include any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon CPKG information of the present invention.

A variety of data processor programs and formats can be used to store the CPKG information of the present invention on computer readable medium. For example, the polynucleotide sequence corresponding to the CPKGs can be represented in a word processing text file, formatted in commercially-available software such as WordPerfect and Microsoft Word, or represented in the form of an ASCII file, stored in a database application, such as DB2, Sybase, Oracle, or the like. Any number of data processor structuring formats (e.g., text file or database) may be adapted in order to obtain computer readable medium having recorded thereon the CPKG information of the present invention.

By providing the CPKG information of the invention in computer readable form, one can routinely access the CPKG sequence information for a variety of purposes. For example, one skilled in the art can use the nucleotide or amino acid sequences of the invention in computer readable form to compare a target sequence or target structural motif with the sequence information stored within the data storage means. Search means are used to identify fragments or regions of the sequences of the invention which match a particular target sequence or target motif.

Arrays and Biochips

The invention also includes an array comprising a panel of CPKGs of the present invention. The array can be used to assay expression of one or more genes in the array.

It will be appreciated by one skilled in the art that the panels of CPKGs of the invention may conveniently be provided on solid supports, as a biochip. For example, polynucleotides may be coupled to an array (e.g., a biochip using GeneChip® for hybridization analysis), to a resin (e.g., a resin which can be packed into a column for column chromatography), or a matrix (e.g., a nitrocellulose matrix for northern blot analysis). The immobilization of molecules complementary to the CPKG(s), either covalently or noncovalently, permits a discrete analysis of the presence or activity of each CPKG in a sample. In an array, for example, polynucleotides complementary to each member of a panel of CPKGs may individually be attached to different, known locations on the array. The array may be hybridized with, for example, polynucleotides extracted from a blood or tissue sample from a subject. The hybridization of polynucleotides from the sample with the array at any location on the array can be detected, and thus the presence or quantity of the CPKG and CPKG transcripts in the sample can be ascertained. In one embodiment, an array based on a biochip is employed. Similarly, Western analyses may be performed on immobilized antibodies specific for CPKPPs hybridized to a protein sample from a subject.

It will also be apparent to one skilled in the art that the entire CPKG product (protein or polynucleotide) molecule need not be conjugated to the biochip support; a portion of the CPKG product or sufficient length for detection purposes (i.e., for hybridization), for example a portion of the CPKG product which is 7, 10, 15, 20, 25, 30, 35, 40, 45, 50, 55, 60, 65, 70, 75, 100 or more nucleotides or amino acids in length, may be sufficient for detection purposes.

In one embodiment, the array can be used to assay gene expression in a tissue to ascertain tissue specificity of genes in the array. In this manner, up to about 12,000 genes can be simultaneously assayed for expression. This allows an expression profile to be developed showing a battery of genes specifically expressed in one or more tissues at a given point in time.

In addition to such qualitative determination, the invention allows the quantitation of gene expression in the biochip. Thus, not only tissue specificity, but also the level of expression of a battery of CPKGs in the tissue is ascertainable. Thus, CPKGs can be grouped on the basis of their tissue expression per se and level of expression in that tissue. Normal levels of expression can be determined using cancer-free samples. The determination of normal levels of expression is useful, for example, in ascertaining the relationship of gene expression between or among tissues. Thus, one tissue or cell type can be perturbed and the effect on gene expression in a second tissue or cell type can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the arrays can be used to monitor the time course of expression of one or more genes in the array. This can occur in various biological contexts, such as development and differentiation, disease progression and cellular transformation and activation.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

Importantly, the invention provides arrays useful for ascertaining differential expression patterns of one or more genes identified in diseased tissue versus non-diseased tissue. This provides a battery of genes that serve as a molecular target for diagnosis or therapeutic intervention. In particular, biochips can be made comprising arrays not only of the CPKGs listed in Table 1, but of CPKGs specific to subjects suffering from specific manifestations or stages of the disease (i.e., metastasized vs. non-metastasized cancer).

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant any material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, etc. In general, the substrates allow optical detection and have low background fluorescence.

Generally the substrate is planar, although as will be appreciated by those in the art, other configurations of substrates may be used as well. For example, the probes may be placed on the inside surface of a tube, for flow-through sample analysis to minimize sample volume. Similarly, the substrate may be flexible, such as a flexible foam, including closed cell foams made of particular plastics.

In one embodiment, the surface of the biochip and the probe may be derivatized with chemical functional groups for subsequent attachment of the two. Thus, for example, the biochip is derivatized with a chemical functional group including, but are not limited to, amino groups, carboxy groups, oxo groups and thiol groups. Using these functional groups, the probes can be attached using functional groups on the probes. For example, nucleic acids containing amino groups can be attached to surfaces comprising amino groups using homo-or hetero-bifunctional linkers. In addition, in some cases, additional linkers, such as alkyl groups (including substituted and heteroalkyl groups) may be used.

In an embodiment, the oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In an additional embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface. For example, photoactivation techniques utilizing photopolymerization compounds and techniques are used. In one embodiment, the nucleic acids can be synthesized in situ, using well-known photolithographic techniques. In one embodiment, a substantial portion of the polynucleotide probes stably attached to a nucleic acid array of the present invention can hybridize under stringent conditions to RNA transcripts of cancer genes, or the complements thereof. For instance, at least 15%, 20%, 25%, 30%, 35%, 40%, 45%, or 50% of the polynucleotide probes on the nucleic acid array can hybridize to cancer genes.

The present invention also contemplates polypeptide arrays. In one embodiment, a substantial portion of the polypeptides that are stably associated with a polypeptide array of the present invention are antibodies specific for polypeptides encoded by cancer genes. In many examples, these antibodies constitute at least 15%, 20%, 25%, 30%, 35%, 40%, 45%, or 50% of the total polypeptides attached to the polypeptide array.

Modifications to the above-described compositions and methods of the invention, according to standard techniques, will be readily apparent to one skilled in the art and are meant to be encompassed by the invention.

This invention is further illustrated by the following examples which should not be construed as limiting.

### EXAMPLES

### Example 1

### Two-Tier Statistical Analysis of Gene Expression Data

The two-tier statistical analysis approach can be described in the following way. Sample sets are created for the four cancers listed above along with the corresponding normal tissues. The gene expression data generated from the Affymetrix MG U95 microarray set for each of the tissue sample types is “extracted” from the Gene Logic BioExpress™ database. The number of samples in each of the sample sets is in the range of 25 to over 100. The number of different genes whose expression is monitored by these arrays is in the range of 40,000-50,000.

The first step in the statistical analysis was to do a contrast analysis in the expression data for the above described sample sets. This method used the results of a one-way analysis of variance (ANOVA) on the individual sample sets. Unlike a simple t-test, which would compare the mean expression for each gene across a two-sample set, a contrast analysis compared the relative levels of the mean expression of each gene for the eight samples sets (described above) to a specified pattern. This pattern was defined as “high in the tumor sample set, low in the normal sample set.” As in the case with a two-group t-test, a ranking score (t-score) was generated to characterize how well a pattern matches the data. The analysis was done in a way such that ranking the genes for the comparisons in the decreasing order of t-score gave the same order as ranking the gene in increasing order of p-value. The actual contrast analysis was carried out with the Contrast Tool algorithm presented in the Gene Logic gx2000 analysis suite. A complete description of this algorithm can be found in the GeneExpress® 2000 Users Manual. Table 5A lists the U95 probe set names (qualifiers) that met the specified contrast pattern with a p-value equal to or smaller than 0.01 for the eight sample sets defined in the above paragraph. Table 5B provides the corresponding gene name for each qualifier in Table 5A.

The second step in the two-tier approach was to perform a simple t-test on each pair of cancer and normal tissues samples and identify the genes that show a statistical difference in expression with a p-value that was equal to or smaller than 0.01. This analysis was executed with the Fold Change Analysis tool in the Gene Logic gx200O analysis suite. A complete description of this algorithm can be found in the GeneExpress® 2000 Users Manual. The p-value of the simple t-test for each gene in Table 5A on each pair of cancer and normal tissue samples is depicted in Table 6A. Genes with p-values of no greater than 0.01 in at least two of the four sample sets of the major cancer types listed above are identified in Table 6B.

### Example 2

### Transmembrane Hidden Markov Model (TMHMM) Analysis

The TMHMM profiles of the polypeptides encoded by the CPKGs were generated using the TMHMM algorithm described by Krogh et al., J. Mol. Biol., 305:567-580, 2001.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

TABLE 5A
Contrast Analysis of Gene Expression in Cancer and Cancer-Free Samples

| | | | BLAST Hits And | | |
|---|---|---|---|---|---|
| | | GenBank | Warnings.Ref sequence | | |
| On Chip | Qualifier | Accession No. | ID | T Score | P-Value |
| HG_U95A | 36174_at | X70326 | NM_023009 | 16.0300 | 0.0000 |
| HG_U95A | 40690_at | X54942 | NM_001827 | 12.9000 | 0.0000 |
| HG_U95A | 35699_at | AF053306 | NM_001211 | 11.4700 | 0.0000 |
| HG_U95A | 1721_g_at | U65410 | NM_002358 | 11.4600 | 0.0000 |
| HG_U95A | 38618_at | AC002073 | | 10.9400 | 0.0000 |
| HG_U95A | 1942_s_at | U37022 | NM_000075, NM_032913 | 10.8400 | 0.0000 |
| HG_U95E | 91194_at | AF154332 | | 10.5500 | 0.0000 |
| HG_U95A | 572_at | M86699 | NM_003318 | 10.0200 | 0.0000 |
| HG_U95A | 40129_at | U47077 | NM_023936 | 10.0100 | 0.0000 |
| HG_U95A | 38847_at | D79997 | NM_014791 | 9.6900 | 0.0000 |
| HG_U95A | 40788_at | U84371 | NM_001625 | 9.6600 | 0.0000 |
| HG_U95A | 34852_g_at | AF011468 | NM_003158, NM_003600 | 9.6400 | 0.0000 |
| HG_U95A | 910_at | M15205 | NM_003258 | 9.5900 | 0.0000 |
| HG_U95A | 40915_r_at | Y00272 | | 9.5300 | 0.0000 |
| HG_U95B | 51141_at | AI949781 | NM_021158 | 9.5100 | 0.0000 |
| HG_U95A | 33324_s_at | D88357 | NM_001786 | 9.3600 | 0.0000 |
| HG_U95A | 37310_at | X02419 | NM_002658 | 8.7500 | 0.0000 |
| HG_U95A | 1100_at | L76191 | NM_001569 | 8.7100 | 0.0000 |
| HG_U95A | 37677_at | V00572 | NM_000291 | 8.6000 | 0.0000 |
| HG_U95A | 34851_at | AF011468 | NM_003158, NM_003600 | 8.4200 | 0.0000 |
| HG_U95A | 480_at | U56816 | NM_004203 | 8.2500 | 0.0000 |
| HG_U95A | 366_s_at | Z29066 | NM_002497 | 8.1600 | 0.0000 |
| HG_U95A | 1225_g_at | X66363 | NM_006201 | 8.0800 | 0.0000 |
| HG_U95A | 1803_at | X05360 | NM_001786 | 7.9900 | 0.0000 |
| HG_U95A | 31873_at | U52112 | NM_003491 | 7.9700 | 0.0000 |
| HG_U95A | 1031_at | U09564 | NM_003137 | 7.8600 | 0.0000 |
| HG_U95A | 33266_at | AF015254 | NM_004217 | 7.5600 | 0.0000 |
| HG_U95A | 38920_at | AF016582 | NM_001274 | 7.3100 | 0.0000 |
| HG_U95A | 36004_at | AF074382 | NM_003639 | 7.3100 | 0.0000 |
| HG_U95A | 33317_at | L20320 | NM_001799 | 7.2500 | 0.0000 |
| HG_U95A | 33559_at | U61412 | NM_005975 | 7.0700 | 0.0000 |
| HG_U95E | 88045_at | AI345571 | | 6.9700 | 0.0000 |
| HG_U95A | 975_at | Y13115 | NM_014264 | 6.9300 | 0.0000 |
| HG_U95C | 62248_at | R80823 | | 6.9000 | 0.0000 |
| HG_U95A | 39183_at | X66363 | NM_006201 | 6.8700 | 0.0000 |
| HG_U95A | 1224_at | X66363 | NM_006201 | 6.8000 | 0.0000 |
| HG_U95A | 37228_at | U01038 | NM_005030 | 6.7900 | 0.0000 |
| HG_U95A | 1250_at | U47077 | | 6.6800 | 0.0000 |
| HG_U95A | 35714_at | U89606 | NM_003681 | 6.6600 | 0.0000 |
| HG_U95A | 41374_at | AB016869 | NM_003952 | 6.4700 | 0.0000 |
| HG_U95A | 594_s_at | M55265 | NM_001895 | 6.3900 | 0.0000 |
| HG_U95A | 37238_s_at | AF014118 | NM_004203 | 6.3100 | 0.0000 |
| HG_U95B | 47096_at | AA161293 | | 6.2400 | 0.0000 |
| HG_U95A | 32378_at | M26252 | NM_002654 | 6.1900 | 0.0000 |
| HG_U95A | 40549_at | L04658 | NM_004935 | 5.9900 | 0.0000 |
| HG_U95A | 32081_at | AB023166 | | 5.9600 | 0.0000 |
| HG_U95A | 40645_at | L33801 | NM_002093 | 5.8600 | 0.0000 |
| HG_U95E | 91445_at | AI560159 | | 5.7100 | 0.0000 |
| HG_U95A | 168_at | U50196 | NM_001123, NM_006721 | 5.6900 | 0.0000 |
| HG_U95A | 41384_at | AF117829 | | 5.6100 | 0.0000 |
| HG_U95B | 52888_at | AA456454 | | 5.5200 | 0.0000 |
| HG_U95C | 55889_at | W72923 | NM_016364 | 5.4000 | 0.0000 |
| HG_U95E | 73710_at | AI475805 | | 5.3700 | 0.0000 |
| HG_U95A | 905_at | L76200 | NM_000858 | 5.3100 | 0.0000 |
| HG_U95A | 33208_at | U28424 | NM_006260 | 5.2900 | 0.0000 |
| HG_U95A | 32799_at | AF023268 | NM_005698 | 5.2500 | 0.0000 |
| HG_U95D | 68350_s_at | AI589365 | NM_004327, NM_021574 | 5.1100 | 0.0000 |
| HG_U95A | 37229_at | U49844 | NM_001184 | 5.0700 | 0.0000 |
| HG_U95B | 53647_at | AL037995 | | 5.0200 | 0.0000 |
| HG_U95A | 31670_s_at | U81554 | NM_001222, NM_006947 | 4.9900 | 0.0000 |
| HG_U95A | 40966_at | AF099989 | NM_013233 | 4.9800 | 0.0000 |
| HG_U95A | 38819_at | U33635 | NM_002821 | 4.9800 | 0.0000 |
| HG_U95A | 1823_g_at | HG4677-HT5102 | | 4.8600 | 0.0000 |
| HG_U95E | 73996_at | AA912743 | | 4.8300 | 0.0000 |
| HG_U95A | 31488_s_at | S81916 | NM_000291 | 4.8100 | 0.0000 |
| HG_U95A | 1752_at | AD000092 | NM_004343 | 4.8100 | 0.0000 |
| HG_U95D | 88282_at | AA765234 | | 4.8000 | 0.0000 |
| HG_U95A | 1809_at | AB003698 | NM_003503 | 4.7900 | 0.0000 |
| HG_U95B | 55644_at | R49183 | | 4.7700 | 0.0000 |
| HG_U95A | 1108_s_at | M18391 | NM_005232 | 4.7600 | 0.0000 |
| HG_U95A | 1438_at | X75208 | NM_004443 | 4.7500 | 0.0000 |
| HG_U95A | 1064_at | U02680 | NM_002822 | 4.7400 | 0.0000 |
| HG_U95A | 799_at | X80343 | NM_003885 | 4.7200 | 0.0000 |
| HG_U95A | 36718_s_at | L42452 | NM_005391 | 4.7100 | 0.0000 |
| HG_U95A | 175_s_at | U33053 | NM_002741 | 4.6300 | 0.0000 |
| HG_U95A | 36117_at | L13616 | | 4.6200 | 0.0000 |
| HG_U95A | 33642_s_at | U17986 | NM_005629 | 4.5700 | 0.0000 |
| HG_U95A | 1792_g_at | M68520 | | 4.5400 | 0.0000 |
| HG_U95D | 79789_at | AI221234 | | 4.5100 | 0.0000 |
| HG_U95A | 41506_at | AF032437 | NM_003668 | 4.3300 | 0.0100 |
| HG_U95A | 33245_at | AF004709 | | 4.3200 | 0.0100 |
| HG_U95A | 39173_at | X56597 | NM_001436 | 4.2800 | 0.0100 |
| HG_U95A | 35694_at | AB014587 | NM_004834 | 4.2800 | 0.0100 |
| HG_U95A | 1082_at | M34667 | NM_002660 | 4.2800 | 0.0000 |
| HG_U95A | 33814_at | AF005046 | NM_005884 | 3.9900 | 0.0000 |
| HG_U95E | 71762_at | AI630528 | | 3.8800 | 0.0000 |

TABLE 5B
Contrast Analysis of Gene Expression in Cancer and Cancer-Free Samples

| Qualifier | Known Gene Name |
|---|---|
| 36174_at | macrophage myristoylated alanine-rich C kinase substrate |
| 40690_at | CDC28 protein kinase 2 |
| 35699_at | budding uninhibited by benzimidazoles 1 (yeast homolog), beta |
| 1721_g_at | MAD2 (mitotic arrest deficient, yeast, homolog)-like 1 |
| 38618_at | |
| 1942_s_at | cyclin-dependent kinase 4 |
| 91194_at | |
| 572_at | TTK protein kinase |
| 40129_at | protein kinase, DNA-activated, catalytic polypeptide, hypothetical protein MGC2616 |
| 38847_at | KIAA0175 gene product |
| 40788_at | adenylate kinase 2 |
| 34852_g_at | serine/threonine kinase 6, serine/threonine kinase 15 |
| 910_at | thymidine kinase 1, soluble |
| 40915_r_at | cell division cycle 2, G1 to S and G2 to M |
| 51141_at | protein kinase domains containing protein similar to phosphoprot |
| 33324_s_at | cell division cycle 2, G1 to S and G2 to M |
| 37310_at | plasminogen activator, urokinase |
| 1100_at | interleukin-1 receptor-associated kinase 1 |
| 37677_at | phosphoglycerate kinase 1 |
| 34851_at | serine/threonine kinase 6, serine/threonine kinase 15 |
| 480_at | membrane-associated tyrosine- and threonine-specific cdc2-inhibi |
| 366_s_at | NIMA (never in mitosis gene a)-related kinase 2 |
| 1225_g_at | PCTAIRE protein kinase 1 |
| 1803_at | cell division cycle 2, G1 to S and G2 to M |
| 31873_at | N-acetyltransferase, homolog of S. cerevisiae ARD1 |
| 1031_at | SFRS protein kinase 1 |
| 33266_at | serine/threonine kinase 12 |
| 38920_at | CHK1 (checkpoint, S. pombe) homolog |
| 36004_at | inhibitor of kappa light polypeptide gene enhancer in B-cells, kinase gamma |
| 33317_at | cyclin-dependent kinase 7 (homolog of Xenopus MO15 cdk-activating kinase) |
| 33559_at | PTK6 protein tyrosine kinase 6 |
| 88045_at | mitogen-activated protein kinase kinase kinase kinase 3 |
| 975_at | serine/threonine kinase 18 |
| 62248_at | v-erb-b2 avian erythroblastic leukemia viral oncogene homolog 3 |
| 39183_at | PCTAIRE protein kinase 1 |
| 1224_at | PCTAIRE protein kinase 1 |
| 37228_at | polo (Drosophia)-like kinase |
| 1250_at | protein kinase, DNA-activated, catalytic polypeptide |
| 35714_at | pyridoxal (pyridoxine, vitamin B6) kinase |
| 41374_at | ribosomal protein S6 kinase, 70 kD, polypeptide 2 |
| 594_s_at | casein kinase 2, alpha 1 polypeptide |
| 37238_s_at | membrane-associated tyrosine- and threonine-specific cdc2-inhibi |
| 47096_at | EphB2 |
| 32378_at | pyruvate kinase, muscle |
| 40549_at | cyclin-dependent kinase 5 |
| 32081_at | citron (rho-interacting, serine/threonine kinase 21) |
| 40645_at | glycogen synthase kinase 3 beta |
| 91445_at | phosphoglycerate kinase 1 |
| 168_at | adenosine kinase |
| 41384_at | receptor-interacting serine-threonine kinase 2 |
| 52888_at | cell division cycle 2-like 1 (PITSLRE proteins) |
| 55889_at | protein phosphatase |
| 73710_at | chymotrypsin-like |
| 905_at | guanylate kinase 1 |
| 33208_at | DnaJ (Hsp40) homolog, subfamily C, member 3 |
| 32799_at | secretory carrier membrane protein 3 |
| 68350_s_at | breakpoint cluster region |
| 37229_at | ataxia telangiectasia and Rad3 related |
| 53647_at | hypothetical protein FLJ21324 |
| 31670_s_at | signal recognition particle 72 kD, calcium/calmodulin-dependent protein kinase (CaM kinase) II |
| | gamma |
| 40966_at | Ste-20 related kinase |
| 38819_at | PTK7 protein tyrosine kinase 7 |
| 1823_g_at | ret proto-oncogene (multiple endocrine neoplasia and medullary thyroid carcinoma 1, Hirschsprung |
| | disease) |
| 73996_at | PTK2 protein tyrosine kinase 2 |
| 31488_s_at | phosphoglycerate kinase 1 |
| 1752_at | calreticulin |
| 88282_at | haspin |
| 1809_at | CDC7 (cell division cycle 7, S. cerevisiae, homolog)-like 1 |
| 55644_at | cyclin-dependent kinase 5, regulatory subunit 1 (p35) |
| 1108_s_at | EphA1 |
| 1438_at | EphB3 |
| 1064_at | protein tyrosine kinase 9 |
| 799_at | cyclin-dependent kinase 5, regulatory subunit 1 (p35) |
| 36718_s_at | pyruvate dehydrogenase kinase, isoenzyme 3 |
| 175_s_at | protein kinase C-like 1 |
| 36117_at | PTK2 protein tyrosine kinase 2 |
| 33642_s_at | solute carrier family 6 (neurotransmitter transporter, creatine), member 8 |
| 1792_g_at | cyclin-dependent kinase 2 |
| 79789_at | |
| 41506_at | mitogen-activated protein kinase-activated protein kinase 5 |
| 33245_at | mitogen-activated protein kinase 13 |
| 39173_at | fibrillarin |
| 35694_at | mitogen-activated protein kinase kinase kinase kinase 4 |
| 1082_at | phospholipase C, gamma 1 (formerly subtype 148) |
| 33814_at | p21(CDKN1A)-activated kinase 4 |
| 71762_at | |

TABLE 6A
Fold Change Analysis of Gene Expression in Cancer and Cancer-Free Samples

| Qualifier | P-value (Colon) | P-value (Lung) | P-value (Breast) | P-value (Prostate) |
|---|---|---|---|---|
| 36174_at | 0.0000 | 0.0000 | 0.0000 | 0.0000 |
| 40690_at | 0.0000 | 0.0000 | 0.0000 | 0.0090 |
| 35699_at | 0.0000 | 0.0000 | 0.0000 | 0.0002 |
| 1721_g_at | 0.0000 | 0.0001 | 0.0000 | 0.2100 |
| 38618_at | 0.0000 | 0.0000 | 0.0000 | 0.0000 |
| 1942_s_at | 0.0000 | 0.0000 | 0.0006 | 0.0021 |
| 91194_at | 0.0000 | 0.0000 | 0.0000 | 0.0034 |
| 572_at | 0.0000 | 0.0000 | 0.0000 | 0.0004 |
| 40129_at | 0.0000 | 0.0000 | 0.0000 | 0.0080 |
| 38847_at | 0.0000 | 0.0000 | 0.0000 | 0.1200 |
| 40788_at | 0.1780 | 0.0000 | 0.0000 | 0.0000 |
| 34852_g_at | 0.0000 | 0.0000 | 0.0000 | 0.1200 |
| 910_at | 0.0005 | 0.0000 | 0.0000 | down |
| 40915_r_at | 0.0000 | 0.0010 | 0.0000 | no change |
| 51141_at | 0.0000 | 0.0120 | 0.0000 | 0.0070 |
| 33324_s_at | 0.0000 | 0.0000 | 0.0000 | 0.0110 |
| 37310_at | 0.0000 | 0.0000 | 0.0000 | down |
| 1100_at | 0.0000 | 0.0000 | 0.0010 | 0.0240 |
| 37677_at | 0.0000 | 0.0000 | 0.0000 | down |
| 34851_at | 0.0000 | 0.0000 | 0.0000 | 0.3000 |
| 480_at | 0.0000 | 0.0000 | 0.0000 | 0.0110 |
| 366_s_at | 0.0000 | 0.0004 | 0.0000 | 0.2420 |
| 1225_g_at | 0.0400 | 0.0002 | 0.0000 | 0.0001 |
| 1803_at | 0.0000 | 0.0000 | 0.0000 | 0.2300 |
| 31873_at | 0.0000 | 0.0031 | 0.0000 | 0.0782 |
| 1031_at | 0.0000 | 0.0000 | 0.0005 | 0.3400 |
| 33266_at | 0.0000 | 0.0000 | 0.0000 | 0.0180 |
| 38920_at | 0.0005 | 0.0200 | 0.0200 | no change |
| 36004_at | 0.0000 | 0.0002 | 0.0000 | 0.2860 |
| 33317_at | 0.0000 | 0.0070 | 0.0100 | 0.0013 |
| 33559_at | 0.0600 | 0.0000 | 0.0000 | 0.0000 |
| 88045_at | 0.0000 | 0.0009 | 0.3803 | 0.0002 |
| 975_at | 0.0000 | 0.0070 | 0.0000 | 0.3000 |
| 62248_at | 0.1400 | 0.0001 | 0.0000 | 0.0000 |
| 39183_at | 0.0700 | 0.0200 | 0.0000 | 0.0002 |
| 1224_at | 0.0300 | 0.0004 | 0.0000 | 0.0089 |
| 37228_at | 0.0000 | 0.0000 | 0.0000 | 0.7100 |
| 1250_at | 0.0000 | 0.0002 | 0.0001 | 0.0600 |
| 35714_at | 0.0000 | 0.0769 | 0.0000 | 0.0650 |
| 41374_at | 0.4000 | 0.0000 | 0.0000 | 0.6000 |
| 594_s_at | 0.0000 | 0.0000 | 0.0030 | 0.0700 |
| 37238_s_at | 0.0010 | 0.0000 | 0.0000 | 0.8000 |
| 47096_at | 0.0000 | 0.0005 | 0.4000 | no change |
| 32378_at | 0.0000 | 0.0000 | 0.0000 | down |
| 40549_at | 0.0000 | 0.0030 | 0.0000 | 0.2700 |
| 32081_at | 0.0001 | 0.0030 | 0.0000 | 0.3000 |
| 40645_at | 0.0010 | 0.7000 | 0.0000 | 0.0400 |
| 91445_at | 0.0056 | 0.0008 | 0.0000 | 0.3790 |
| 168_at | 0.0006 | 0.0143 | 0.0008 | 0.0089 |
| 41384_at | 0.0500 | 0.9000 | 0.0000 | 0.0080 |
| 52888_at | 0.6000 | 0.0007 | 0.9000 | 0.0700 |
| 55889_at | 0.0000 | 0.0120 | 0.2499 | 0.0885 |
| 73710_at | 0.0000 | 0.0002 | 0.0500 | 0.4000 |
| 905_at | 0.0000 | 0.0860 | 0.0000 | 0.8910 |
| 33208_at | 0.4900 | 0.2040 | 0.0102 | 0.1290 |
| 32799_at | 0.0010 | 0.0002 | 0.0000 | 0.0069 |
| 68350_s_at | 0.0008 | 0.0900 | 0.0003 | 0.0300 |
| 37229_at | 0.0000 | 0.0500 | 0.7000 | 0.0060 |
| 53647_at | 0.0000 | 0.0977 | 0.0004 | 0.1999 |
| 31670_s_at | 0.0200 | 0.0400 | 0.0120 | 0.0160 |
| 40966_at | 0.7000 | 0.0002 | 0.4000 | 0.0003 |
| 38819_at | 0.0000 | 0.0200 | 0.0120 | 0.1200 |
| 1823_g_at | 0.5000 | 0.1000 | 0.0000 | 0.3000 |
| 73996_at | 0.0030 | 0.0300 | 0.0000 | 0.7000 |
| 31488_s_at | 0.0000 | 0.0003 | 0.0000 | 0.9000 |
| 1752_at | 0.8660 | 0.0840 | 0.0000 | 0.1012 |
| 88282_at | 0.0000 | 0.0003 | 0.1000 | 0.0050 |
| 1809_at | 0.0008 | 0.0004 | 0.0000 | 0.9775 |
| 55644_at | 0.0000 | 0.0380 | 0.0000 | 0.1080 |
| 1108_s_at | 0.0000 | 0.0040 | 0.5000 | 0.0400 |
| 1438_at | 0.0000 | 0.0110 | 0.1100 | 0.1550 |
| 1064_at | 0.7000 | 0.0000 | 0.0400 | 0.0900 |
| 799_at | 0.7128 | 0.0167 | 0.5880 | 0.0366 |
| 36718_s_at | 0.0002 | 0.0000 | 0.0000 | 0.2800 |
| 175_s_at | 0.0500 | 0.0300 | 0.0000 | 0.0000 |
| 36117_at | 0.0002 | 0.5000 | 0.0080 | 0.0090 |
| 33642_s_at | 0.4000 | 0.0060 | 0.0000 | 0.1400 |
| 1792_g_at | 0.0000 | 0.0100 | 0.0030 | 0.1400 |
| 79789_at | 0.0160 | down | 0.0000 | 0.2520 |
| 41506_at | 0.0098 | 0.0021 | 0.0960 | 0.0700 |
| 33245_at | 0.9200 | 0.0000 | 0.0000 | 0.0370 |
| 39173_at | 0.0000 | 0.0001 | 0.0000 | 0.0200 |
| 35694_at | 0.0000 | 0.2080 | 0.9200 | 0.1460 |
| 1082_at | 0.0004 | 0.0126 | 0.0820 | 0.6196 |
| 33814_at | down | down | 0.0000 | 0.0005 |
| 71762_at | 0.4391 | 0.1822 | 0.0000 | 0.1425 |

TABLE 6B
Genes Differentially Expressed in Two or More Major Cancers Relative to Cancer-Free Tissues

| Qualifier | Known Gene Name |
|---|---|
| 36174_at | macrophage myristoylated alanine-rich C kinase substrate |
| 40690_at | CDC28 protein kinase 2 |
| 35699_at | budding uninhibited by benzimidazoles 1 (yeast homolog), beta |
| 1721_g_at | MAD2 (mitotic arrest deficient, yeast, homolog)-like 1 |
| 38618_at | |
| 1942_s_at | cyclin-dependent kinase 4 |
| 91194_at | |
| 572_at | TTK protein kinase |
| 40129_at | protein kinase, DNA-activated, catalytic polypeptide, hypothetical protein MGC2616 |
| 38847_at | KIAA0175 gene product |
| 40788_at | adenylate kinase 2 |
| 34852_g_at | serine/threonine kinase 6, serine/threonine kinase 15 |
| 910_at | thymidine kinase 1, soluble |
| 40915_r_at | cell division cycle 2, G1 to S and G2 to M |
| 51141_at | protein kinase domains containing protein similar to phosphoprot |
| 33324_s_at | cell division cycle 2, G1 to S and G2 to M |
| 37310_at | plasminogen activator, urokinase |
| 1100_at | interleukin-1 receptor-associated kinase 1 |
| 37677_at | phosphoglycerate kinase 1 |
| 34851_at | serine/threonine kinase 6, serine/threonine kinase 15 |
| 480_at | membrane-associated tyrosine- and threonine-specific cdc2-inhibi |
| 366_s_at | NIMA (never in mitosis gene a)-related kinase 2 |
| 1225_g_at | PCTAIRE protein kinase 1 |
| 1803_at | cell division cycle 2, G1 to S and G2 to M |
| 31873_at | N-acetyltransferase, homolog of S. cerevisiae ARD1 |
| 1031_at | SFRS protein kinase 1 |
| 33266_at | serine/threonine kinase 12 |
| 36004_at | inhibitor of kappa light polypeptide gene enhancer in B-cells, kinase gamma |
| 33317_at | cyclin-dependent kinase 7 (homolog of Xenopus MO15 cdk-activating kinase) |
| 33559_at | PTK6 protein tyrosine kinase 6 |
| 88045_at | mitogen-activated protein kinase kinase kinase kinase 3 |
| 975_at | serine/threonine kinase 18 |
| 62248_at | v-erb-b2 avian erythroblastic leukemia viral oncogene homolog 3 |
| 39183_at | PCTAIRE protein kinase 1 |
| 1224_at | PCTAIRE protein kinase 1 |
| 37228_at | polo (Drosophia)-like kinase |
| 1250_at | protein kinase, DNA-activated, catalytic polypeptide |
| 35714_at | pyridoxal (pyridoxine, vitamin B6) kinase |
| 41374_at | ribosomal protein S6 kinase, 70 kD, polypeptide 2 |
| 594_s_at | casein kinase 2, alpha 1 polypeptide |
| 37238_s_at | membrane-associated tyrosine- and threonine-specific cdc2-inhibi |
| 47096_at | EphB2 |
| 32378_at | pyruvate kinase, muscle |
| 40549_at | cyclin-dependent kinase 5 |
| 32081_at | citron (rho-interacting, serine/threonine kinase 21) |
| 40645_at | glycogen synthase kinase 3 beta |
| 91445_at | phosphoglycerate kinase 1 |
| 168_at | adenosine kinase |
| 41384_at | receptor-interacting serine-threonine kinase 2 |
| 73710_at | chymotrypsin-like |
| 905_at | guanylate kinase 1 |
| 32799_at | secretory carrier membrane protein 3 |
| 68350_s_at | breakpoint cluster region |
| 37229_at | ataxia telangiectasia and Rad3 related |
| 53647_at | hypothetical protein FLJ21324 |
| 40966_at | Ste-20 related kinase |
| 73996_at | PTK2 protein tyrosine kinase 2 |
| 31488_s_at | phosphoglycerate kinase 1 |
| 88282_at | haspin |
| 1809_at | CDC7 (cell division cycle 7, S. cerevisiae, homolog)-like 1 |
| 55644_at | cyclin-dependent kinase 5, regulatory subunit 1 (p35) |
| 1108_s_at | EphA1 |
| 36718_s_at | pyruvate dehydrogenase kinase, isoenzyme 3 |
| 175_s_at | protein kinase C-like 1 |
| 36117_at | PTK2 protein tyrosine kinase 2 |
| 33642_s_at | solute carrier family 6 (neurotransmitter transporter, creatine), member 8 |
| 1792_g_at | cyclin-dependent kinase 2 |
| 41506_at | mitogen-activated protein kinase-activated protein kinase 5 |
| 33245_at | mitogen-activated protein kinase 13 |
| 39173_at | fibrillarin |
| 33814_at | p21(CDKN1A)-activated kinase 4 |

1. A method, comprising the steps of:
detecting an expression profile of at least one gene in a biological sample of a subject; and
comparing said expression profile to a reference expression profile of said at least one gene,
wherein said at least one gene is differentially expressed in at least two types of cancer cells as compared to corresponding cancer-free cells.

2. The method of claim 1, wherein each of said at least two types is selected from the group consisting of colon cancer, lung cancer, breast cancer, and prostate cancer.

3. The method of claim 2, wherein said at least one gene includes at least one kinase gene which is overexpressed in said at least two types of cancer cells as compared to said corresponding cancer-free cells.

4. The method of claim 2, wherein said at least one gene includes one or more genes selected from Table 1.

5. The method of claim 2, wherein the biological sample is a colon sample, a lung sample, a breast sample, or a prostate sample, and said reference expression profile is an average expression profile of said at least one gene in reference biological samples of cancer-free subjects.

6. The method of claim 5, wherein said expression profile and said reference expression profile are determined using RT-PCR, nucleic acid arrays, or immunoassays.

7. The method of claim 2, wherein said subject has colon cancer, lung cancer, breast cancer, or prostate cancer.

8. A method comprising:
detecting an expression profile of at least one gene in a biological sample of a subject; and
comparing said expression profile to a reference expression profile of said at least one gene,
wherein said at least one gene has a statistically significant T score under a contrast analysis, wherein the contrast analysis is capable of comparing average expression levels of said at least one gene in at least four sample sets to a predetermined pattern, wherein said at least four sample sets include a first pair and a second pair of sample sets, the first pair of sample sets including a set of samples having a first cancer and a set of samples free of the first cancer, the second pair of sample sets including a set of samples having a second cancer and a set of samples free of the second cancer, and wherein said predetermined pattern is defined as “high in cancer sample set, low in cancer-free set” or “low in cancer sample set, high in cancer-free set.”

9. A method, comprising the steps of:
detecting in a biological sample the level of T cells that are activated by one or more polypeptides encoded by at least one gene which is differentially expressed in at least two types of cancer cells as compared to corresponding cancer-free cells; and
comparing the level to a reference level of said T cells.

10. A pharmaceutical composition comprising a pharmaceutically acceptable carrier and at least one component selected from the group consisting of:
a polypeptide encoded by a gene which is over-expressed in at least two types of cancer cells as compared to corresponding cancer-free cells;
a variant of said polypeptide; and
a polynucleotide encoding said polypeptide or said variant.

11. The pharmaceutical composition of claim 10, wherein the pharmaceutical composition is a vaccine formulation capable of eliciting an immune response against a cancer cell or a component thereof, and wherein said gene is selected from Table 1.

12. A method comprising administering an immunoeffective amount of the pharmaceutical composition of claim 11 to a subject in need thereof.

13. A pharmaceutical composition comprising a pharmaceutically acceptable carrier and at least one component selected from the group consisting of:
an agent capable of modulating the expression of a gene which is over-expressed in at least two types of cancer cells as compared to corresponding cancer-free cells;
an agent capable of binding to, or modulating an activity of, a polypeptide encoded by said gene; and
a T cell activated by said polypeptide.

14. The pharmaceutical composition of claim 13, wherein said component is selected from the group consisting of:
a polynucleotide comprising or encoding an RNA that is capable of inhibiting or decreasing the expression of said gene by RNA interference or an antisense mechanism;
an antibody specific for said polypeptide encoded by said gene; and
an inhibitor of a biological activity of said polypeptide, wherein said gene is selected from Table 1.

15. A method comprising administering the pharmaceutical composition of claim 14 to a subject who has colon cancer, lung cancer, breast cancer, or prostate cancer.

16. The pharmaceutical composition of claim 13, wherein said component is a polynucleotide comprising or encoding a siRNA sense or antisense sequence selected from Table 4.

17. A nucleic acid array comprising one or more substrate supports which are stably associated with polynucleotide probes, wherein a substantial portion of all polynucleotide probes that are stably associated with said one or more substrate supports are capable of hybridizing under reduced stringent, stringent or highly stringent conditions to RNA transcripts, or the complements thereof, of genes which are differentially expressed in at least two types of cancer cells as compared to corresponding cancer-free cells.

18. A polypeptide array comprising one or more substrate supports which are stably associated with a plurality of polypeptides, wherein a substantial portion of all polypeptides that are stably associated with said one or more substrate supports consists of:
polypeptides encoded by genes which are differentially expressed in at least two types of cancer cells as compared to corresponding cancer-free cells;
variants of said polypeptides;
antibodies specific for said polypeptides or variants;
or any combination of said polypeptides, variants or antibodies.

19. A cancer diagnostic kit comprising at least one of:
a polynucleotide probe capable of specifically binding to a sequence recited in any one of SEQ ID NOS:1-44 or the complement thereof; and
an antibody capable of specifically binding to a polypeptide sequence recited in any one of SEQ ID NOS:45-88.

20. A method for identifying an agent capable of modulating an activity of a gene which is differentially expressed in at least two types of cancer cells as compared to corresponding cancer-free cells, said method comprising:
contacting a candidate agent with a polypeptide encoded by said gene;
comparing a biological activity of said polypeptide in the presence and absence of said candidate agent to determine if said candidate agent can modulate said biological activity.
